(19) United States
(12) Patent Application Publication
(10) Pub. No.: US 2010/0292222 A1
(43) Pub. Date: Nov. 18, 2010

(54) CHEMICAL COMPOUNDS 751

(75) Inventors: Robert Hugh Bradbury, Cheshire (GB); Alfred Arthur Rabow, Cheshire (GB)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/776,970

(22) Filed: May 10, 2010

Related U.S. Application Data
(60) Provisional application No. 61/177,005, filed on May 11, 2009.
(60) Provisional application No. 61/218,646, filed on Jun. 19, 2009.

(51) Int. Cl. A61K 31/551; C07D 487/04; A61K 31/5025; C07D 243/08; A61P 35/00

(52) U.S. Cl. 514/218; 544/236; 514/248; 540/492

(57) ABSTRACT

The invention concerns bicyclic compounds of Formula (I)

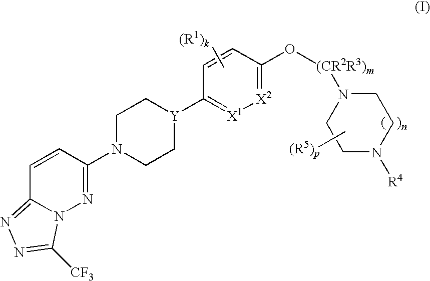

wherein, R¹, R², R³, R⁴, R⁵, X¹, X², Y, k, m, n and p are as defined in the description. The present invention also relates to processes for the preparation of such compounds, pharmaceutical compositions containing them and their use in the treatment of androgen-receptor associated conditions, particularly prostate cancer.

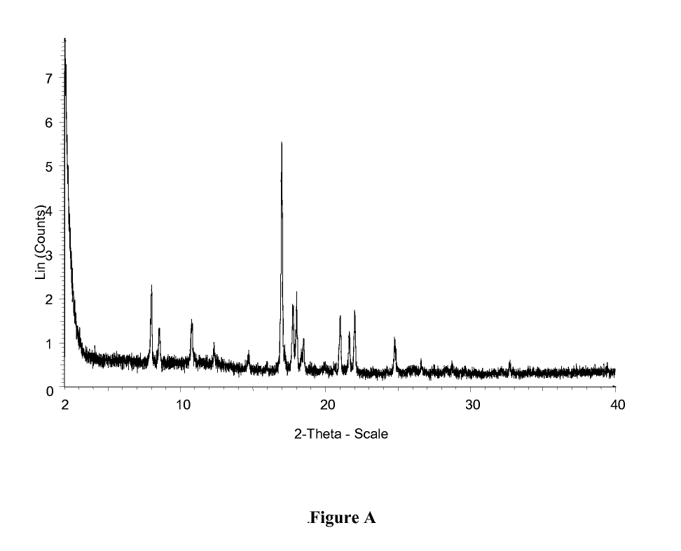

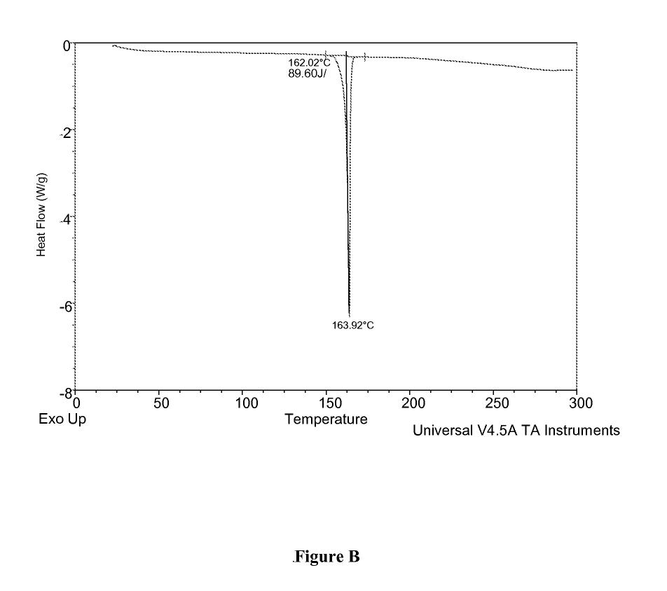

This application claims the benefit under 35 U.S.C. §119(e) of Application No. 61/177,005 filed 11 May 2009 and Application No. 61/218,646 filed 19 Jun. 2009.

This invention relates to new bicyclic derivatives and, more particularly, to bicyclic derivatives that act as ligands of the androgen receptor (AR). This invention also relates to methods for the preparation of such bicyclic derivatives, and novel intermediates in the preparation thereof, to pharmaceutical compositions containing such bicyclic derivatives, to the use of such bicyclic derivatives in the preparation of medicines, and to the use of such bicyclic derivatives in the treatment of androgen-receptor associated condition such as prostate cancer.

Prostate cancer is the second most common cause of death from cancer amongst men in developed countries and is projected to account for 25% of incident cases diagnosed and 9% of deaths due to cancer, accounting for over 27,000 deaths in the USA in 2009 (A. Jemal et al., CA Cancer J Clin published online May 2009).

The early stages of prostate cancer tumour growth are androgen-dependent and as such respond well to hormonal therapies aimed at causing androgen depletion by surgical (orchidectomy) or medical castration (e.g. LHRH-Agonists (Zoladex™, buserelin), LHRH antagonists (cetrorelix), or 5α-reductase inhibitors (finasteride)). These treatments are now often used in combination with androgen antagonists (e.g. Casodex™, cyproterone acetate, flutamide) to achieve total androgen blockade. The introduction of androgen deprivation therapy represented a major advance in prostate cancer treatment, however whilst highly effective initially in the majority of patients the cancer will recur within 2-3 years. This recurrence marks the transition of the cancer to a so-called castrate resistant state, where the tumour continues to grow in the presence of low circulating testosterone and no longer responds to classical androgen antagonists. Castrate resistant prostate cancer is a largely unmet medical need with a 5 year survival rate of less than 15%. Docetaxal is currently the only treatment shown to improve survival, offering a benefit of 2 months (O, Smaletz and H. I Scher, Semin. Urol. Oncol., 2002, 20:155-163; D. A. Loblaw et al., J. Clin. Oncol., 2007, 25: 1596-1605).

There is now a body of evidence from both clinical and pre-clinical studies to support the notion that androgen receptor signalling is important in the majority of castrate resistant prostate cancers. The androgen receptor belongs to the family of steroid hormone receptors, which function as transcription factors. The binding of an androgen to the androgen receptor results in the stabilisation of the receptor and protects it form undergoing a rapid proteolytic degradation. The complex of androgen and androgen receptor is transported into the nucleus, where it regulates the expression of androgen responsive genes by binding to their androgen response DNA elements in the promoter region of such androgen responsive genes (D. J. Lamb et al. Vitam. Horm. 2001, 62, 199-230).

It is now well established that the majority of castrate resistant tumours possess a functional androgen receptor, which is frequently mutated or amplified. Receptor mutations arise in approximately 25-30% of patients treated with antagonists and can lead to a promiscuous receptor that recognises androgen antagonists as agonists or is stimulated by other steroids such as glucocorticoids. Gene amplification and over-expression of the androgen receptor is a common finding in castrate resistant cancers and leads to hypersentitivity to low levels of androgens. Pre-clinically the receptor is often over-expressed in in vitro and in vivo models of castrate resistant prostate cancer. Over expression of the receptor can convert hormone responsive lines to hormone refractory, and removal of the androgen receptor using siRNA prevents the growth of an androgen-independent xenograft model, data which support the critical role that this receptor plays in progression from androgen dependent to androgen resistant disease (B. J. Feldman; D. Feldman., Nat Rev Cancer, 2001, 1, 34-45; Chen et al, Curr Opin Pharmacol., 2008, 8, 440-8).

The identification of antiandrogens that would inhibit not only the natural form of the androgen receptor but also its mutated forms and thereby so alter the receptor molecule so that it became unstable would be very useful in the treatment of prostate tumours at various stages of growth. Such compounds could inhibit a recurrence of tumour growth or at least prolong the disease free interval. In the case of oestrogen receptors, such ligands have been identified that destabilise the receptor and lead to a reduction in the receptor content both in vitro and in vivo (S. Dauvois et al., Proc Natl. Acad. Sci. USA, 1992, 89, 4037-41; R. A. McClelland et al. Eur. J. Cancer, 1996, 32A, 413-416). In the case of the androgen receptor, a series of bicyclic derivatives capable of inducing cellular down-regulaton of the androgen receptor in vitro has been described in PCT/GB2008/051206. Nonetheless, there is still a requirement for further compounds having high levels of androgen receptor potency coupled with favourable physical properties, for example, good aqueous solubility, high levels of permeability and/or low levels of plasma protein binding.

A further series of bicyclic derivatives capable of inducing cellular down-regulation of the androgen receptor is described herein. According to a first aspect of the present invention there is therefore provided a compound of Formula (I), or a pharmaceutically acceptable salt thereof:

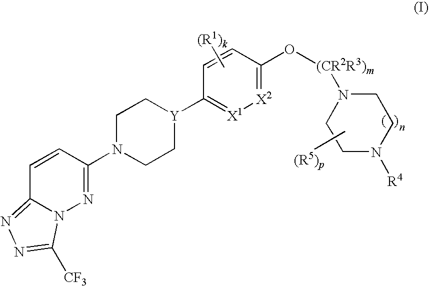

wherein
X¹-X² represents CH—CH, N—CH or CH—N; Y represents N, CH or COH;
R¹, identically or differently on each occurrence, represents halo or C₁₋₆alkyl;
R² and R³, identically or differently on each occurrence, represent hydrogen, methyl, ethyl, isopropyl, cyclopropyl or methoxymethyl;
R⁴ represents C₁₋₆alkyl, C₂₋₆alkanoyl, C₁₋₆alkoxyC₁₋₆alkyl, C₃₋₆cycloalkyl, hydroxyC₂₋₆alkanoyl, C₁₋₆alkoxyC₂₋₆alkanoyl or oxetan-3-ylcarbonyl;
R⁵ represents oxo, methyl, ethyl, isopropyl, cyclopropyl or methoxymethyl;
k represents 0, 1 or 2;
m represents 1, 2, 3 or 4;
n represents 1 or 2; and
p represents 0, 1 or 2;
with the proviso that the compound of Formula (I) is other than:
6-(4-{4-[3-(4-methylpiperazin-1-yl)propoxy]phenyl}piperidin-1-yl)-3-(trifluoromethyl)[1,2,4]triazolo[4,3-b]pyridazine;
6-(4-{4-[2-(4-methylpiperazin-1-yl)ethoxy]phenyl}piperidin-1-yl)-3-(trifluoromethyl)[1,2,4]triazolo[4,3-b]pyridazine;
6-[4-[4-[2-(4-acetylpiperazin-1-yl)ethoxy]phenyl]piperidin-1-yl]-3-(trifluoromethyl)[1,2,4]triazolo[4,3-b]pyridazine;
6-[4-[4-[3-(4-acetylpiperazin-1-yl)propoxy]phenyl]piperidin-1-yl]-3-(trifluoromethyl)[1,2,4]triazolo[4,3-b]pyridazine; or
4-[4-[2-(4-acetylpiperazin-1-yl)ethoxy]phenyl]-1-[3-(trifluoromethyl)[1,2,4]triazolo[4,3-b]pyridazin-6-yl]piperidin-4-ol.

According to a second aspect of the present invention there is provided a compound of Formula (I) or a pharmaceutically acceptable salt thereof

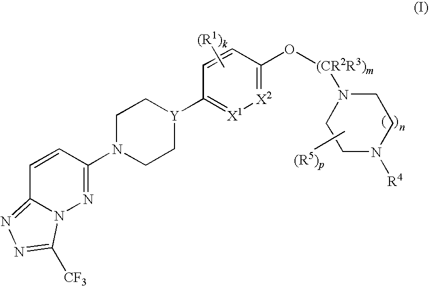

wherein
X¹-X² represents CH—CH, N—CH or CH—N;
Y represents N, CH or COH;
R¹, identically or differently on each occurrence, represents halo or C₁₋₆alkyl;
R² and R³, identically or differently on each occurrence, represent hydrogen, methyl, ethyl, isopropyl, cyclopropyl or methoxymethyl;
R⁴ represents C₁₋₆alkyl, C₂₋₆alkanoyl, C₁₋₆alkoxyC₁₋₆alkyl, C₃₋₆cycloalkyl, hydroxyC₂₋₆alkanoyl, C₁₋₆alkoxyC₂₋₆alkanoyl or oxetan-3-ylcarbonyl;
R⁵ represents oxo, methyl, ethyl, isopropyl, cyclopropyl or methoxymethyl;
k represents 0, 1 or 2;
m represents 1, 2, 3 or 4.
n represents 1 or 2; and
p represents 1 or 2.

According to a further aspect of the invention there is a compound of Formulae (Ia), (Ib) or (Ic), or a pharmaceutically acceptable salt thereof:

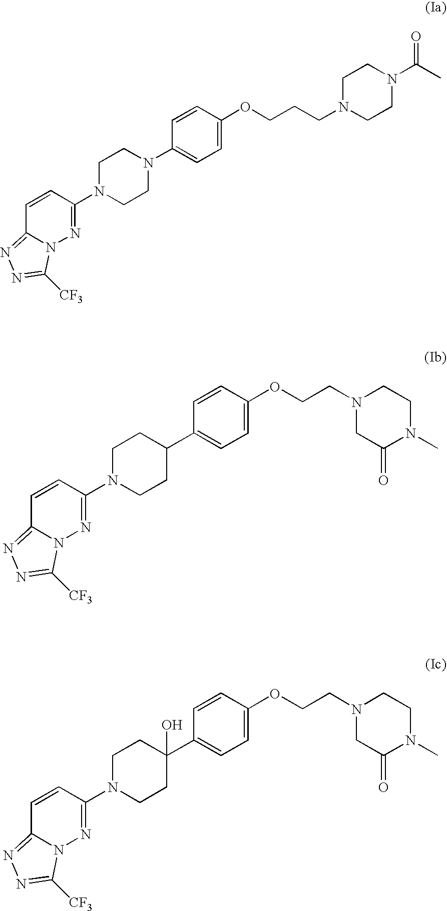

It is to be understood that, insofar as certain of the compounds of Formula (I) defined above may exist in optically active or racemic forms by virtue of one or more asymmetric carbon atoms, the invention includes in its definition any such optically active or racemic form which possesses the above-mentioned activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

It is to be understood that certain compounds of Formulae (I), (Ia), (Ib) or (Ic) above may exist in unsolvated forms as well as solvated forms, such as, for example, hydrated forms. It is to be understood that the present invention encompasses all such solvated forms that possess androgen receptor ligand activity. In one embodiment of the invention, there is therefore provided a compound of Formulae (I), (Ia), (Ib) or (Ic) in a solvated form. In one embodiment of the invention, there is therefore provided a compound of Formulae (I), (Ia), (Ib) or (Ic) in a hydrated form.

It is also to be understood that certain compounds of Formulae (I), (Ia), (Ib) or (Ic) may exist in crystalline form and exhibit polymorphism. The present invention encompasses all such polymorphic forms which possess androgen receptor ligand activity. In one embodiment of the invention, there is therefore provided a compound of Formulae (I), (Ia), (Ib) or (Ic) or a pharmaceutically acceptable salt thereof, in crystalline form.

The term “halo” is used herein to denote fluoro, chloro, bromo and iodo.

An oxo group, when bonded to a carbon atom, replaces two hydrogen atoms on a carbon atom of the parent system. Thus, if a CH₂ group is substituted by oxo, i.e. by a doubly bonded oxygen atom, it becomes a CO group.

The term “C₁₋₆alkyl” is intended to mean a saturated carbon chain of 1 to 6 carbon atoms in length which may be straight-chained or branched. However references to individual alkyl groups such as “propyl” are specific for the straight chain version only and references to individual branched-chain alkyl groups such as tert-butyl are specific for the branched chain version only. For example, “C₁₋₆alkyl” includes, but is not limited to, methyl, ethyl, propyl, isopropyl, butyl, tert-butyl, pentyl, tent-pentyl, hexyl and isohexyl. The term “C₁₋₄alkyl” is to be construed accordingly.

The term “C₃₋₆cycloalkyl” is intended to mean a saturated 3 to 6 membered monocyclic carbon ring. For example “C₃₋₆cycloalkyl” includes cyclopropyl, cyclobutyl, cyclopentyl and cyclohexyl.

The term “C₁₋₆ alkoxy” is intended to mean a saturated carbon chain of 1 to 6 carbon atoms in length, which may be straight-chained or branched, linked to oxygen. For example, “C₁₋₆ alkoxy” includes, but is not limited to, methoxy, ethoxy, propoxy, butoxy, pentoxy and hexoxy.

The term “C₂₋₆alkanoyl” is intended to mean a saturated carbon chain of 1 to 5 carbon atoms in length, which may be straight-chained, branched or cyclic, linked to carbonyl. For example, “C₂₋₆alkanoyl” includes, but is not limited to, acetyl, propanoyl, butanoyl, pentanoyl, hexanoyl, cyclopropylcarbonyl and cyclobutylcarbonyl.

The term “C₁₋₆alkoxyC₁₋₆alkyl” is intended to mean a saturated carbon chain of 1 to 6 carbon atoms in length, which may be straight-chained or branched, linked via oxygen to another saturated carbon chain of 1 to 6 carbon atoms in length, which may be straight-chained or branched. For example, “C₁₋₆alkoxyC₁₋₆alkyl” includes, but is not limited to, methoxyethyl, methoxypropyl, ethoxypropyl, propoxyethyl and butoxypropyl.

The term “hydroxyC₂₋₆alkanoyl” is intended to mean a saturated carbon chain of 1 to 5 carbon atoms in length, which may be straight-chained, branched or cyclic, linked to carbonyl wherein one of the hydrogen atoms of the saturated carbon chain has been replaced by a hydroxy group. For example, “hydroxyC₂₋₆alkanoyl” includes, but is not limited to, hydroxyacetyl, 2-hydroxypropanoyl, 3-hydroxybutanoyl, 4-hydroxypentanoyl, 5-hydroxyhexanoyl, and 3-hydroxy-cyclobutylcarbonyl.

The term “C₁₋₆ alkoxyC₂₋₆alkanoyl” is intended to mean a saturated carbon chain of 1 to 6 carbon atoms in length, which may be straight-chained or branched, linked via oxygen to a C₂₋₆alkanoyl group as defined hereinbefore. For example, “C₁₋₆ alkoxyC₂₋₆alkanoyl” includes, but is not limited to, methoxyacetyl, 2-methoxypropanoyl, 3-methoxybutanoyl, 4-ethoxypentanoyl, 5-ethoxyhexanoyl, and 3-methoxy-cyclobutylcarbonyl.

In further embodiments of the first or second aspects of the present invention, each of the following definitions of X¹, X², Y, R¹, R², R³, R⁴, R⁵, k, m, n and p in paragraphs (1) to (36) hereinafter may be used individually or in combination with one or more of the other following definitions to limit the broadest definition of Formula (I). For example, the skilled person would understand that paragraphs (1), (9), (18), (27), (29) and (32) could be combined to provided a compound of Formula (I), or a pharmaceutically acceptable salt thereof, wherein X¹-X² represents CH—CH, R² and R³ identically on each occurrence both represent hydrogen, R⁴ represents methyl or acetyl, k represents 0, m represents 2 or 3 and n represents 1. Similarly, paragraphs (1), (5), (18), (25), (29), (32) and (34) could be combined to provided a compound of Formula (I), or a pharmaceutically acceptable salt thereof, wherein X¹-X² represents CH—CH, Y represents CH, R⁴ represents methyl or acetyl, R⁵ is bonded to a ring carbon atom adjacent to the nitrogen atom linked to R⁴, m represents 2 or 3, n represents 1 and p represents 0 or 1. Similarly paragraphs (15), (24), (25) and (34) or (14), (23), (25) and (34) could be combined.
(1) X¹-X² represents CH—CH;
(2) X¹-X² represents N—CH;
(3) X¹-X² represents CH—N;
(4) Y represents N;
(5) Y represents CH;
(6) Y represents COH;
(7) R¹ represents methyl;
(8) R¹ represents fluoro;
(9) R² and R³ identically on each occurrence both represent hydrogen;
(10) R² and R³, identically or differently on each occurrence, represent hydrogen or methyl;
(11) R² and R³, identically or differently on each occurrence, represent hydrogen or methoxymethyl;
(12) R⁴ represents methyl, ethyl, isopropyl, cyclopropyl, methoxyethyl, acetyl, hydroxyacetyl, 2-hydroxypropanoyl, methoxyacetyl, 2-methoxypropanoyl;
(13) R⁴ represents C₁₋₆alkyl, C₂₋₆alkanoyl, hydroxyC₂₋₆alkanoyl or C₁₋₆alkoxyC₂₋₆alkanoyl;
(14) R⁴ represents C₁₋₆alkyl;
(15) R⁴ represents C₂₋₆alkanoyl, hydroxyC₂₋₆alkanoyl or C₁₋₆alkoxyC₂₋₆alkanoyl;
(16) R⁴ represents C₂₋₆alkanoyl;
(17) R⁴ represents methyl, ethyl or acetyl
(18) R⁴ represents methyl or acetyl;
(19) R⁴ represents methyl;
(20) R⁴ represents acetyl;
(21) R⁴ represents ethyl;
(22) R⁵ represents oxo or methyl;
(23) R⁵ represents oxo;
(24) R⁵ represents methyl;
(25) R⁵ is bonded to a ring carbon atom adjacent to the nitrogen atom linked to R⁴;
(26) R⁵ is bonded to a ring carbon atom adjacent to the nitrogen atom linked to (CR²R³)ₘ;
(27) k represents 0;
(28) k represents 1;
(29) m represents 2 or 3;
(30) m represents 2;
(31) m represents 3;
(32) n represents 1;
(33) n represents 2;
(34) p represents 0 or 1;
(35) p represents 0;
(36) p represents 1.

Particular novel compounds of Formula (I) include, but are not limited to, the following compounds:
6-(4-{4-[3-(4-acetylpiperazin-1-yl)propoxy]phenyl}piperazin-1-yl)-3-(trifluoromethyl)[1,2,4]triazolo[4,3-b]pyridazine;
1-methyl-4-[2-(4-{1-[3-(trifluoromethyl)[1,2,4]triazolo[4,3-b]pyridazin-6-yl]piperidin-4-yl}phenoxy)ethyl]piperazin-2-one;
4-[2-(4-{4-hydroxy-1-[3-(trifluoromethyl)[1,2,4]triazolo[4,3-b]pyridazin-6-yl]piperidin-4-yl}phenoxy)ethyl]-1-methylpiperazin-2-one;
1-methyl-4-[2-(4-{4-[3-(trifluoromethyl)[1,2,4]triazolo[4,3-b]pyridazin-6-yl]piperazin-1-yl}phenoxy)ethyl]piperazin-2-one;
1-cyclopropyl-4-[2-(4-{4-[3-(trifluoromethyl)[1,2,4]triazolo[4,3-b]pyridazin-6-yl]piperazin-1-yl}phenoxy)ethyl]piperazin-2-one;
1-cyclopropyl-4-[2-(4-{1-[3-(trifluoromethyl)[1,2,4]triazolo[4,3-b]pyridazin-6-yl]piperidin-4-yl}phenoxy)ethyl]piperazin-2-one;
4-methyl-1-[2-(4-{4-[3-(trifluoromethyl)[1,2,4]triazolo[4,3-b]pyridazin-6-yl]piperazin-1-yl}phenoxy)ethyl]piperazin-2-one;
4-methyl-1-[2-(4-{1-[3-(trifluoromethyl)[1,2,4]triazolo[4,3-b]pyridazin-6-yl]piperidin-4-yl}phenoxy)ethyl]piperazin-2-one;
1-methyl-4-[3-(4-{1-[3-(trifluoromethyl)[1,2,4]triazolo[4,3-b]pyridazin-6-yl]piperidin-4-yl}phenoxy)propyl]piperazin-2-one;
1-cyclopropyl-4-[3-(4-{1-[3-(trifluoromethyl)[1,2,4]triazolo[4,3-b]pyridazin-6-yl]piperidin-4-yl}phenoxy)propyl]piperazin-2-one;
1-methyl-4-{2-[(5-{1-[3-(trifluoromethyl)[1,2,4]triazolo[4,3-b]pyridazin-6-yl]piperidin-4-yl}pyridin-2-yl)oxy]ethyl}piperazin-2-one;
4-[2-(2-fluoro-4-{4-hydroxy-1-[3-(trifluoromethyl)[1,2,4]triazolo[4,3-b]pyridazin-6-yl]piperidin-4-yl}phenoxy)ethyl]-1-methylpiperazin-2-one;
4-[2-(2-fluoro-4-{1-[3-(trifluoromethyl)[1,2,4]triazolo[4,3-b]pyridazin-6-yl]piperidin-4-yl}phenoxy)ethyl]-1-methylpiperazin-2-one;
6-(4-{-4-[2-(4-acetyl-1,4-diazepan-1-yl)ethoxy]phenyl}piperidin-1-yl)-3-(trifluoromethyl)[1,2,4]triazolo[4,3-b]pyridazine;
1-ethyl-4-[3-(4-{1-[3-(trifluoromethyl)[1,2,4]triazolo[4,3-b]pyridazin-6-yl]piperidin-4-yl}phenoxy)propyl]piperazin-2-one;
1-(1-methylethyl)-4-[3-(4-{1-[3-(trifluoromethyl)[1,2,4]triazolo[4,3-b]pyridazin-6-yl]piperidin-4-yl}phenoxy)propyl]piperazin-2-one;
1-(2-methoxyethyl)-4-[3-(4-{1-[3-(trifluoromethyl)[1,2,4]triazolo[4,3-b]pyridazin-6-yl]piperidin-4-yl}phenoxy)propyl]piperazin-2-one;
1-ethyl-4-[2-(4-{1-[3-(trifluoromethyl)[1,2,4]triazolo[4,3-b]pyridazin-6-yl]piperidin-4-yl}phenoxy)ethyl]piperazin-2-one;
1-(1-methylethyl)-4-[2-(4-{1-[3-(trifluoromethyl)[1,2,4]triazolo[4,3-b]pyridazin-6-yl]piperidin-4-yl}phenoxy)ethyl]piperazin-2-one;
1-(2-methoxyethyl)-4-[2-(4-{1-[3-(trifluoromethyl)[1,2,4]triazolo[4,3-b]pyridazin-6-yl]piperidin-4-yl}phenoxy)ethyl]piperazin-2-one;
2-oxo-2-{4-[2-(4-{1-[3-(trifluoromethyl)[1,2,4]triazolo[4,3-b]pyridazin-6-yl]piperidin-4-yl}phenoxy)ethyl]piperazin-1-yl}ethanol;
6-[4-(4-{2-[4-(methoxyacetyl)piperazin-1-yl]ethoxy}phenyl)piperidin-1-yl]-3-(trifluoromethyl)[1,2,4]triazolo[4,3-b]pyridazine;
(2S)-1-oxo-1-{4-[2-(4-{1-[3-(trifluoromethyl)[1,2,4]triazolo[4,3-b]pyridazin-6-yl]piperidin-4-yl}phenoxy)ethyl]piperazin-1-yl}propan-2-ol;
(2R)-1-oxo-1-{4-[2-(4-{1-[3-(trifluoromethyl)[1,2,4]triazolo[4,3-b]pyridazin-6-yl]piperidin-4-yl}phenoxy)ethyl]piperazin-1-yl}propan-2-ol;
6-{4-[4-(2-{4-[(2S)-2-methoxypropanoyl]piperazin-1-yl}ethoxy)phenyl]piperidin-1-yl}-3-(trifluoromethyl)[1,2,4]triazolo[4,3-b]pyridazine;
6-(4-{4-[(1R)-2-(4-acetylpiperazin-1-yl)-1-methylethoxy]phenyl}piperidin-1-yl)-3-(trifluoromethyl)[1,2,4]triazolo[4,3-b]pyridazine;
6-(4-{4-[(1S)-2-(4-acetylpiperazin-1-yl)-1-methylethoxy]phenyl}piperidin-1-yl)-3-(trifluoromethyl)[1,2,4]triazolo[4,3-b]pyridazine;
6-(4-{4-[(1S)-2-(4-acetylpiperazin-1-yl)-1-(methoxymethyl)ethoxy]phenyl}piperidin-1-yl)-3-(trifluoromethyl)[1,2,4]triazolo[4,3-b]pyridazine;
6-(4-{4-[(1R)-2-(4-acetylpiperazin-1-yl)-1-(methoxymethyl)ethoxy]phenyl}piperidin-1-yl)-3-(trifluoromethyl)[1,2,4]triazolo[4,3-b]pyridazine;
4-{4-[2-(4-acetylpiperazin-1-yl)ethoxy]-2-methylphenyl}-1-[3-(trifluoromethyl)[1,2,4]triazolo[4,3-b]pyridazin-6-yl]piperidin-4-ol;
4-[2-(4-{4-hydroxy-1-[3-(trifluoromethyl)[1,2,4]triazolo[4,3-b]pyridazin-6-yl]piperidin-4-yl}-3-methylphenoxy)ethyl]-1-methylpiperazin-2-one;
4-methyl-1-[2-(4-{1-[3-(trifluoromethyl)[1,2,4]triazolo[4,3-b]pyridazin-6-yl]piperidin-4-yl}phenoxy)ethyl]-1,4-diazepan-5-one;
6-[4-(4-{2-[(3S)-4-acetyl-3-methylpiperazin-1-yl]ethoxy}phenyl)piperidin-1-yl]-3-(trifluoromethyl)[1,2,4]triazolo[4,3-b]pyridazine;
6-[4-(4-{2-[(3R)-4-acetyl-3-methylpiperazin-1-yl]ethoxy}phenyl)piperidin-1-yl]-3-(trifluoromethyl)[1,2,4]triazolo[4,3-b]pyridazine;
4-{4-[2-(4-acetylpiperazin-1-yl)ethoxy]-2-fluorophenyl}-1-[3-(trifluoromethyl)[1,2,4]triazolo[4,3-b]pyridazin-6-yl]piperidin-4-ol;
4-[2-(3-fluoro-4-{4-hydroxy-1-[3-(trifluoromethyl)[1,2,4]triazolo[4,3-b]pyridazin-6-yl]piperidin-4-yl}phenoxy)ethyl]-1-methylpiperazin-2-one;
1-ethyl-4-[2-(4-{4-hydroxy-1-[3-(trifluoromethyl)[1,2,4]triazolo[4,3-b]pyridazin-6-yl]piperidin-4-yl}phenoxy)ethyl]piperazin-2-one;
1-cyclopropyl-4-[2-(4-{4-hydroxy-1-[3-(trifluoromethyl)[1,2,4]triazolo[4,3-b]pyridazin-6-yl]piperidin-4-yl}phenoxy)ethyl]piperazin-2-one;
4-(4-{2-[(3R)-4-acetyl-3-methylpiperazin-1-yl]ethoxy}phenyl)-1-[3-(trifluoromethyl) [1,2,4]triazolo[4,3-b]pyridazin-6-yl]piperidin-4-ol;
4-(4-{2-[(3S)-4-acetyl-3-methylpiperazin-1-yl]ethoxy}phenyl)-1-[3-(trifluoromethyl) [1,2,4]triazolo[4,3-b]pyridazin-6-yl]piperidin-4-ol;
4-{4-[2-(4-acetyl-1,4-diazepan-1-yl)ethoxy]-2-fluorophenyl}-1-[3-(trifluoromethyl)[1,2,4]triazolo[4,3-b]pyridazin-6-yl]piperidin-4-ol;
4-[3-(3-fluoro-4-{4-hydroxy-1-[3-(trifluoromethyl)[1,2,4]triazolo[4,3-b]pyridazin-6-yl]piperidin-4-yl}phenoxy)propyl]-1-methylpiperazin-2-one;
4-{4-[2-(4-acetylpiperazin-1-yl)propoxy]phenyl}-1-[3-(trifluoromethyl)[1,2,4]triazolo[4,3-b]pyridazin-6-yl]piperidin-4-ol;
4-{4-[2-(4-acetyl-1,4-diazepan-1-yl)propoxy]phenyl}-1-[3-(trifluoromethyl) [1,2,4]triazolo[4,3-b]pyridazin-6-yl]piperidin-4-ol;

and pharmaceutically acceptable salts thereof.

According to one aspect of the invention the compound of Formula (I) is 4-[2-(4-{4-hydroxy-1-[3-(trifluoromethyl)[1,2,4]triazolo[4,3-b]pyridazin-6-yl]piperidin-4-yl}phenoxy)ethyl]-1-methylpiperazin-2-one or a pharmaceutically acceptable salt thereof.

According to one aspect of the invention the compound of Formula (I) is 4-{4-[2-(4-acetylpiperazin-1-yl)ethoxy]-2-methylphenyl}-1-[3-(trifluoromethyl)[1,2,4]triazolo[4,3-b]pyridazin-6-yl]piperidin-4-ol or a pharmaceutically acceptable salt thereof.

According to one aspect of the invention the compound of Formula (I) is 4-[2-(4-{4-hydroxy-1-[3-(trifluoromethyl)[1,2,4]triazolo[4,3-b]pyridazin-6-yl]piperidin-4-yl}-3-methylphenoxy)ethyl]-1-methylpiperazin-2-one or a pharmaceutically acceptable salt thereof.

According to one aspect of the invention the compound of Formula (I) is 4-[2-(3-fluoro-4-{4-hydroxy-1-[3-(trifluoromethyl)[1,2,4]triazolo[4,3-b]pyridazin-6-yl]piperidin-4-yl}phenoxy)ethyl]-1-methylpiperazin-2-one or a pharmaceutically acceptable salt thereof.

According to one aspect of the invention the compound of Formula (I) is 1-ethyl-4-[2-(4-{4-hydroxy-1-[3-(trifluoromethyl)[1,2,4]triazolo[4,3-b]pyridazin-6-yl]piperidin-4-yl}phenoxy)ethyl]piperazin-2-one or a pharmaceutically acceptable salt thereof.

A suitable pharmaceutically-acceptable salt of a compound of Formulae (I), (Ia), (Ib) or (Ic) is, for example, an acid-addition salt of a compound of Formulae (I), (Ia), (Ib) or (Ic) for example an acid-addition salt with an inorganic or organic acid such as hydrochloric, hydrobromic, sulphuric, trifluoroacetic, citric, maleic acid, naphthalene-1,5-disulfonic, toluene-4-sulfonic or fumaric acid; or, for example, a salt of a compound of the Formulae (I), (Ia), (Ib) or (Ic) which is sufficiently acidic, for example an alkali or alkaline earth metal salt such as a calcium or magnesium salt, or an ammonium salt, or a salt with an organic base such as methylamine, dimethylamine, trimethylamine, piperidine, morpholine or tris-(2-hydroxyethyl)amine.

The compounds of the invention may be administered in the form of a pro-drug, that is a compound that is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula (I) and in vivo cleavable amide derivatives that may be formed at a carboxy group or an amino group in a compound of the Formula (I).

Accordingly, the present invention includes those compounds of the Formula (I) as defined hereinbefore when made available by organic synthesis and when made available within the human or animal body by way of cleavage of a pro-drug thereof. Accordingly, the present invention includes those compounds of the Formula (I) that are produced by organic synthetic means and also such compounds that are produced in the human or animal body by way of metabolism of a precursor compound, that is a compound of the Formula (I) may be a synthetically-produced compound or a metabolically-produced compound.

A suitable pharmaceutically-acceptable pro-drug of a compound of the Formula (I) is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically-acceptable pro-drug of a compound of the Formula (I) that possesses a carboxy group is, for example, an in vivo cleavable ester thereof. An in vivo cleavable ester of a compound of the Formula (I) containing a carboxy group is, for example, a pharmaceutically-acceptable ester which is cleaved in the human or animal body to produce the parent acid. Suitable pharmaceutically-acceptable esters for carboxy include C₁₋₆alkyl esters such as methyl, ethyl and tent-butyl, C₁₋₆alkoxymethyl esters such as methoxymethyl esters, C₁₋₆alkanoyloxymethyl esters such as pivaloyloxymethyl esters, 3-phthalidyl esters, C₃₋₈cycloalkylcarbonyloxy-C₁₋₆alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆alkoxycarbonyloxy-C₁₋₆alkyl esters such as methoxycarbonyloxymethyl and 1-methoxycarbonyloxyethyl esters.

A suitable pharmaceutically-acceptable pro-drug of a compound of the Formula (I) that possesses a hydroxy group is, for example, an in vivo cleavable ester or ether thereof.

An in vivo cleavable ester or ether of a compound of the Formula (I) containing a hydroxy group is, for example, a pharmaceutically-acceptable ester or ether which is cleaved in the human or animal body to produce the parent hydroxy compound. Suitable pharmaceutically-acceptable ester forming groups for a hydroxy group include inorganic esters such as phosphate esters (including phosphoramidic cyclic esters). Further suitable pharmaceutically-acceptable ester forming groups for a hydroxy group include C₁₋₁₀alkanoyl groups such as acetyl, benzoyl, phenylacetyl and substituted benzoyl and phenylacetyl groups, C₁₋₁₀alkoxycarbonyl groups such as ethoxycarbonyl, N,N-[di-C₁₋₄alkyl]carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-C₁₋₄alkylpiperazin-1-ylmethyl. Suitable pharmaceutically-acceptable ether forming groups for a hydroxy group include α-acyloxyalkyl groups such as acetoxymethyl and pivaloyloxymethyl groups.

A suitable pharmaceutically-acceptable pro-drug of a compound of the Formula (I) that possesses a carboxy group is, for example, an in vivo cleavable amide thereof, for example an amide formed with an amine such as ammonia, a C₁₋₄alkylamine such as methylamine, a di-C₁₋₄alkylamine such as dimethylamine, N-ethyl-N-methylamine or diethylamine, a C₁₋₄alkoxy-C₂₋₄alkylamine such as 2-methoxyethylamine, a phenyl-C₁₋₄alkylamine such as benzylamine and amino acids such as glycine or an ester thereof.

A suitable pharmaceutically-acceptable pro-drug of a compound of the Formula (I) that possesses an amino group is, for example, an in vivo cleavable amide derivative thereof. Suitable pharmaceutically-acceptable amides from an amino group include, for example an amide formed with C₁₋₁₀alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄)alkylpiperazin-1-ylmethyl.

The in vivo effects of a compound of the Formula (I) may be exerted in part by one or more metabolites that are formed within the human or animal body after administration of a compound of the Formula (I). As stated hereinbefore, the in vivo effects of a compound of the Formula (I) may also be exerted by way of metabolism of a precursor compound (a pro-drug).

As stated hereinbefore, certain compounds of Formula (I) may exist in crystalline form and exhibit polymorphism. According to the present invention there is therefore provided a crystalline form of 6-(4-{4-[3-(4-acetylpiperazin-1-yl)propoxy]phenyl}piperazin-1-yl)-3-(trifluoromethyl)[1,2,4]triazolo[4,3-b]pyridazine.

In one embodiment of the present invention, there is provided a crystalline form of 6-(4-{4-[3-(4-acetylpiperazin-1-yl)propoxy]phenyl}piperazin-1-yl)-3-(trifluoromethyl)[1,2,4]triazolo[4,3-b]pyridazine, Form A, which has an X-ray powder diffraction pattern with at least one specific peak at a 2θ value of about 17.0° when measured using CuKa radiation, more particularly wherein said value may be plus or minus 0.5° 2θ.

In one embodiment of the present invention, there is provided a crystalline form of 6-(4-{4-[3-(4-acetylpiperazin-1-yl)propoxy]phenyl}piperazin-1-yl)-3-(trifluoromethyl)[1,2,4]triazolo[4,3-b]pyridazine, Form A, which has an X-ray powder diffraction pattern with at least one specific peak at a 2θ value of about 8.0° when measured using CuKa radiation, more particularly wherein said value may be plus or minus 0.5° 2θ.

In one embodiment of the present invention, there is provided a crystalline form of 6-(4-{4-[3-(4-acetylpiperazin-1-yl)propoxy]phenyl}piperazin-1-yl)-3-(trifluoromethyl)[1,2,4]triazolo[4,3-b]pyridazine, Form A, which has an X-ray powder diffraction pattern with at least two specific peaks at 2θ values of about 17.0° and 8.0° when measured using CuKa radiation, more particularly wherein said value may be plus or minus 0.5° 2θ.

In one embodiment of the present invention, there is provided a crystalline form of 6-(4-{4-[3-(4-acetylpiperazin-1-yl)propoxy]phenyl}piperazin-1-yl)-3-(trifluoromethyl)[1,2,4]triazolo[4,3-b]pyridazine, Form A, which has an X-ray powder diffraction pattern with specific peaks at 2θ values of about 17.0, 8.0, 18.0, 22.0, 17.8, 21.0, 10.8, 8.5, 21.7 and 18.5° when measured using CuKa radiation, more particularly wherein said values may be plus or minus 0.5° 2θ.

In one embodiment of the present invention, there is provided a crystalline form of 6-(4-{4-[3-(4-acetylpiperazin-1-yl)propoxy]phenyl}piperazin-1-yl)-3-(trifluoromethyl)[1,2,4]triazolo[4,3-b]pyridazine, Form A, which has an X-ray powder diffraction pattern substantially the same as the X-ray powder diffraction pattern shown in Figure A when measured using CuKa radiation.

When it is stated that the present invention relates to a crystalline form the degree of crystallinity is conveniently greater than about 60%, more conveniently greater than about 80%, preferably greater than about 90% and more preferably greater than about 95%. Most preferably the degree of crystallinity is greater than about 98%.

It will be understood that the 2θ values of the X-ray powder diffraction pattern may vary slightly from one machine to another or from one sample to another, and so the values quoted are not to be construed as absolute.

It is known in the art that an X-ray powder diffraction pattern may be obtained which has one or more measurement errors depending on measurement conditions (such as equipment, sample preparation or machine used). In particular, it is generally known that intensities in an X-ray powder diffraction pattern may fluctuate depending on measurement conditions and sample preparation. For example, persons skilled in the art of X-ray powder diffraction will realise that the relative intensities of peaks may vary according to the orientation of the sample under test and on the type and setting of the instrument used. The skilled person will also realise that the position of reflections can be affected by the precise height at which the sample sits in the diffractometer and the zero calibration of the diffractometer. The surface planarity of the sample may also have a small effect. Hence a person skilled in the art will appreciate that the diffraction pattern data presented herein is not to be construed as absolute and any crystalline form that provides a power diffraction pattern substantially identical to those disclosed herein fall within the scope of the present disclosure (for further information see Jenkins, R & Snyder, R. L. ‘Introduction to X-Ray Powder Diffractometry’ John Wiley & Sons, 1996).

Generally, a measurement error of a diffraction angle in an X-ray powder diffractogram is approximately plus or minus 0.5° 2-theta, and such degree of a measurement error should be taken into account when considering the X-ray powder diffraction pattern in Figure A and when reading Table A. Furthermore, it should be understood that intensities might fluctuate depending on experimental conditions and sample preparation (preferred orientation). Preferred orientation occurs when there is a tendency for the crystal morphology (shape) to exhibit a particular orientation such as acicular (needle-like), resulting in a non-random orientation of the crystals when sampled for XRPD analysis. This can result in differences in relative intensity of peaks.

### Preparation of Compounds of Formula (I)

Certain processes for the synthesis of compounds of Formula (I) are provided as a further feature of the invention. Thus, according to a further aspect of the invention there is provided a process for the preparation of a compound of Formula (I) or a pharmaceutically acceptable salt thereof, which comprises a process (a), (b), (c), (d), (e), (f) or (g) wherein, unless otherwise defined, the variables are as defined hereinbefore for compounds of Formula (I):

(a) reaction of a compound of Formula (II) with a compound of Formula (III):

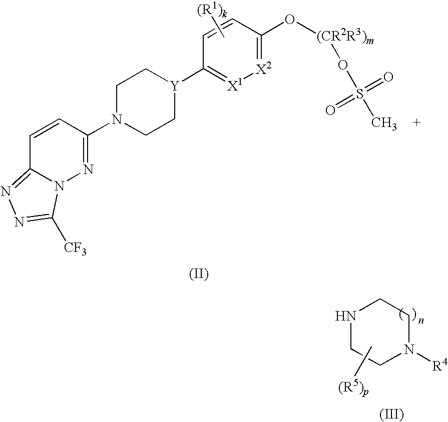

(b) reaction of a compound of Formula (IV) with a compound of Formula (V):

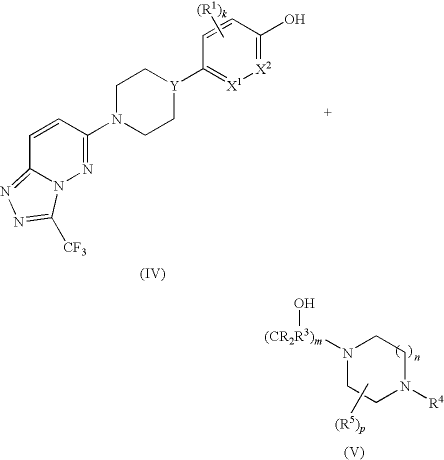

(c) when R⁴ in Formula (I) is C₂₋₆alkanoyl, hydroxyC₂₋₆alkanoyl or C₁₋₆alkoxyC₂₋₆alkanoyl and R⁵ is other than oxo, reaction of a compound of Formula (VI) with the appropriate carboxylic acid, hydroxycarboxylic acid or alkoxycarboxylic acid:

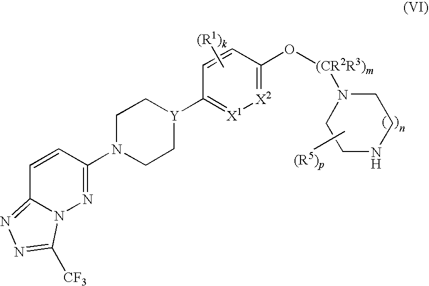

(d) when R⁴ is C₁₋₆alkyl and R⁵ is other than oxo bonded to a ring carbon atom adjacent to the nitrogen atom linked to R⁴, reaction of a compound of Formula (VI) with the appropriate aldehyde, in the presence of a suitable acid and a suitable reducing agent;
(e) when Y is CH, reduction of a compound of Formula (VII):

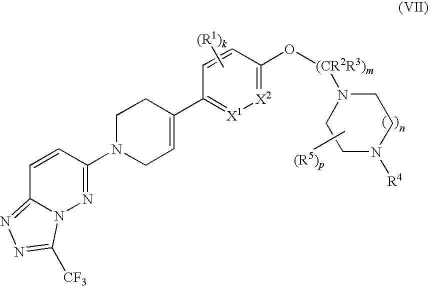

(f) reaction of a compound of Formula (VIII) with a compound of Formula (IX) wherein L represents chloro, bromo or iodo:

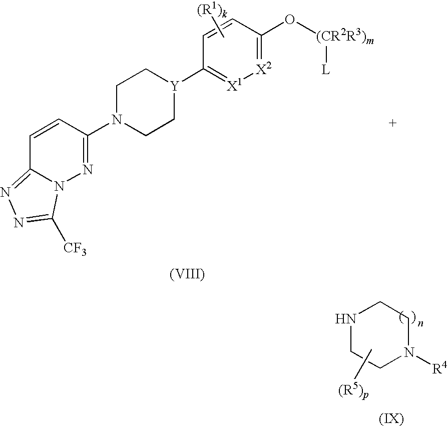

(g) reaction of a compound of Formula (X) with a compound of Formula (XI) wherein L represents chloro, bromo or iodo:

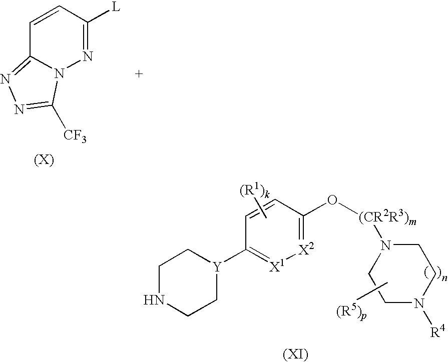

and thereafter, if necessary:
(i) converting a functional group of one compound of the invention into another functional group;
(ii) introducing a new functional group into one compound of the invention;
(iii) removing any protecting groups;
(iv) for compounds of the invention in the form of a single enantiomer separating a racemic compound of the invention into separate enantiomers;
(v) preparing a pharmaceutically acceptable salt thereof; and/or
(vi) preparing a crystalline form thereof.

Specific reaction conditions for processes (a) to (g) above are as follows:

Process (a)—a compound of Formula (II) may be reacted with a compound of Formula (III) in the presence of a suitable base, for example DIPEA, and a suitable solvent, for example DMF or DMA, and at a suitable temperature, for example 50 to 150° C., more suitably about 100° C.

Process (b)—a compound of Formula (IV) may be reacted under nitrogen with a compound of Formula (V) in the presence of triphenylphosphine and a suitable oxidant, for example DIAD, and a suitable solvent, for example THF, and at a suitable temperature, for example 0 to 100° C., more suitably at a temperature from 0° C. to ambient temperature.

Process (c)—a compound of Formula (VI) may be reacted with the appropriate carboxylic acid, hydroxycarboxylic acid or alkoxycarboxylic acid in the presence of a suitable base, for example DIPEA, a suitable coupling agent, for example HATU, and a suitable solvent, for example DMF, and at a suitable temperature, for example 10 to 100° C., more suitably at ambient temperature.

Process (d)—a compound of Formula (VI) may be reacted with the appropriate aldehyde in the presence of a suitable acid, for example acetic acid, a suitable reducing agent, for example sodium triacetoxyhydroborate, and a suitable solvent, for example a mixture of THD, DCM and methanol, and at a suitable temperature, for example 0 to 100° C., more suitably at ambient temperature.

Process (e)—a compound of Formula (VII) may be reduced using a suitable reducing agent, for example ammonium formate and 10% palladium on carbon, in the presence of a suitable solvent, for example ethanol, and at a suitable temperature, for example 50 to 150° C., more suitably about 78° C.

Process (f)—a compound of Formula (VIII) may be reacted with a compound of Formula (IX) optionally in the presence of a suitable base, for example DIPEA, and in the presence of a suitable solvent, for example DMF, and at a suitable temperature, for example 0 to 100° C., more suitably about 50° C.

Process (g)—a compound of Formula (X) may be reacted with a compound of Formula (XI) optionally in the presence of a suitable base, for example DIPEA, and in the presence of a suitable solvent, for example DMF, and at a suitable temperature, for example 0 to 100° C., more suitably about 50° C.

Compounds of Formulae (II) and (IV) can be prepared according to Scheme 1 below wherein all variables are as defined hereinbefore for compounds of Formula (I).

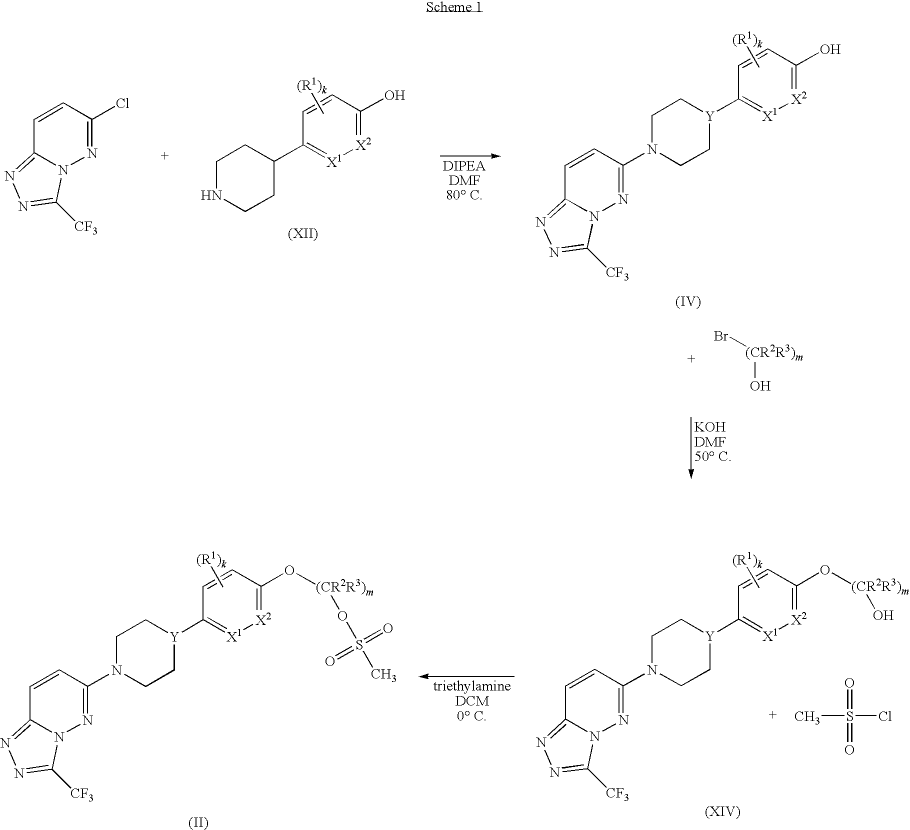

The 6-chloro-3-(trifluoromethyl)-[1,2,4]triazolo[4,3-b]pyridazine used in Scheme 1 may be obtained as described in Monatsh. Chem. (1972), 103, 1591. Compounds of Formulae (XII) and (XIII) are available from commercial sources or may be prepared by methods well-known to those skilled in the art including methods according to or analogous to methods described in the chemical literature. For example, compounds of Formula (XII) wherein Y is COH may be prepared as described in Journal of Medicinal Chemistry (2000), 43(5), 984-994.

Compounds of Formula (V) can be prepared according to Scheme 2 below wherein all variables are as defined hereinbefore for compounds of Formula (I).

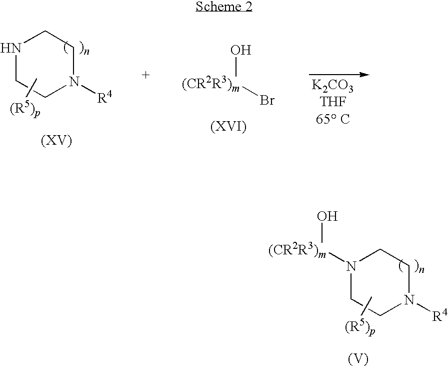

Compounds of Formulae (XV) and (XVI) are available from commercial sources or may be prepared by methods well-known to those skilled in the art including methods according to or analogous to methods described in the chemical literature.

Compounds of Formula (VI) may be prepared using a method analogous to process (b) described above wherein R⁴ is replaced by a suitable protecting group, for example an N-tert-butoxycarbonyl derivative. Once this reaction is complete, the protecting group is removed to provide a compound of Formula (VI). For example, an N-tert-butoxycarbonyl derivative protecting group may be removed by treatment with a suitable acid, such as trifluoroacetic acid.

Compounds of Formula (VII) may be prepared according to Scheme 3 below wherein all variables are as defined hereinbefore for compounds of Formula (I).

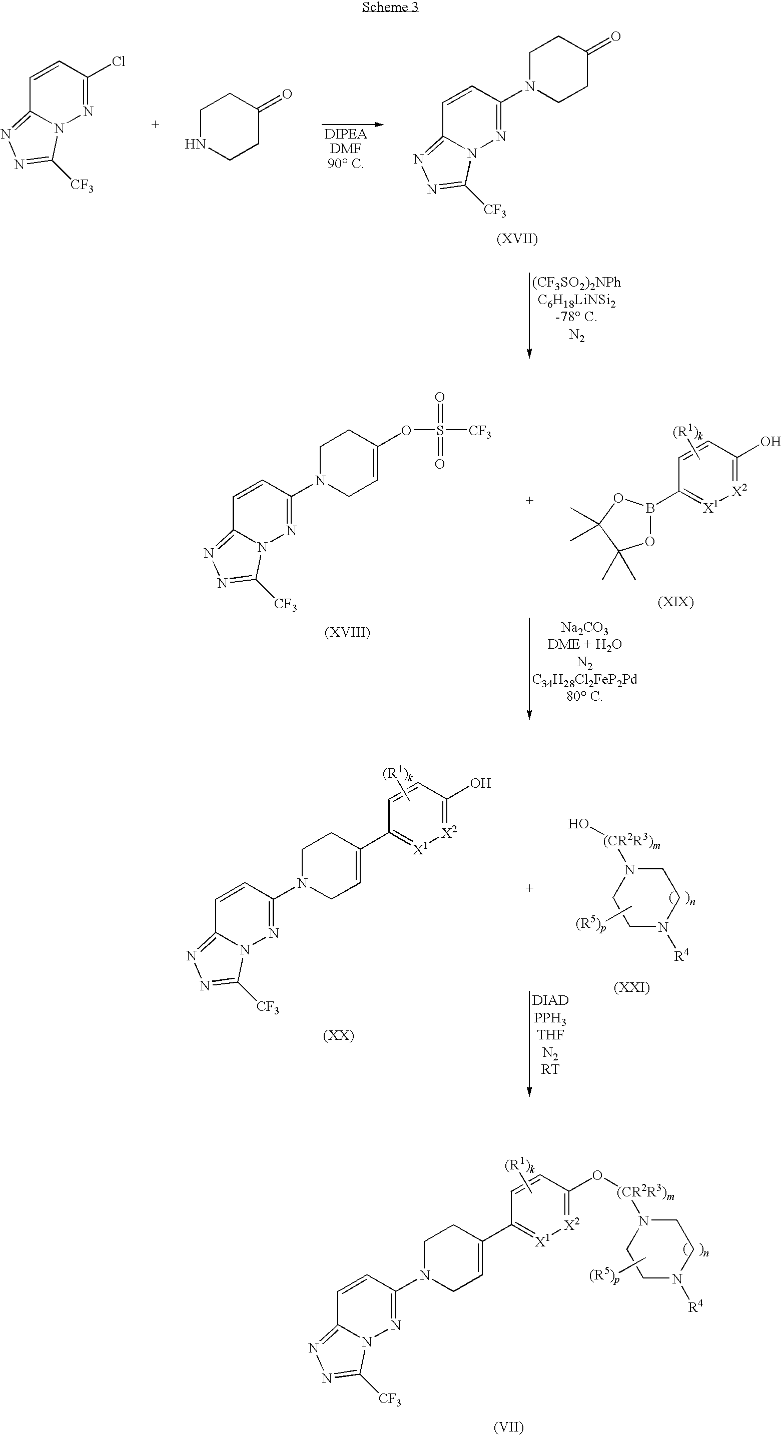

6-Chloro-3-(trifluoromethyl)-[1,2,4]triazolo[4,3-b]pyridazine, 4-piperidone and compounds of Formulae (XIX) and (XXI) are available from commercial sources, may be prepared by methods well-known to those skilled in the art including methods according to or analogous to methods described in the chemical literature, or may be prepared according to methods described in the Examples.

Compounds of Formula (VIII) may be prepared by reacting a compound of Formula (II) with the appropriate lithium halide in a suitable solvent, for example dioxan, and at a suitable temperature, for example 50 to 150° C., more suitably about 100° C.

Compounds of Formulae (X) and (XI) may be prepared by methods well-known to those skilled in the art including methods according to or analogous to methods described in the chemical literature, for example 6-chloro-3-(trifluoromethyl)-[1,2,4]triazolo[4,3-b]pyridazine may be obtained as described in Monatsh. Chem. (1972), 103, 1591.

### Biological Assays

The ability of compounds to reduce Androgen Receptor (AR) numbers was assessed in a cell based immuno-fluorescence assay using the LNCaP prostate epithelial cell line.

### a) LNCaP Androgen Receptor Down-Regulation Cell Assay

This immunofluorescence end point assay measures the ability of a test compound to down-regulate and reduce measured levels of the AR in the LNCaP prostate carcinoma cell line (LNCaP clone FGC (CRL-1740) obtained from the American Type Culture Collection (ATCC)).

LNCaP cells were cultured in Growth Medium (phenol red free Roswell Park Memorial Institute (RPMI) 1640 (Invitrogen Code no. 11835-063) containing 2 mM L-Glutamine (Invitrogen Code no. 25030-024) and 1% (v/v) Penicillin/Streptomycin (10000 units/ml Penicillin and 10000 μg/ml of Streptomycin utilising penicillin G (sodium salt) and streptomycin sulphate: prepared in normal saline, Invitrogen Code no. 15140122) and 10% (v/v) foetal bovine serum (FBS)) in a 5% CO₂ air incubator at 37° C. Cells for assay were harvested from T175 stock flasks by washing once in PBS (phosphate buffered saline, pH 7.4) (Invitrogen Code no. 14190-094) and harvested using 5 mls of 1× Trypsin/ethylaminediaminetetraacetic acid (EDTA) (10× Trypsin-EDTA, 5.0 g/L Trypsin, 2.0 g/L of EDTA•4Na and 8.5 g/L of NaCl, without Phenol Red, Invitrogen Code no. 15400-054) diluted in PBS solution. A 5 ml volume of Growth Medium was added to each flask (as above except that 5% (v/v) charcoal stripped FBS (HyClone Code no. SH30068.03) was included instead of 10% (v/v) FBS). Cells were syringed at least twice using a sterile 18G×1.5″ (1.2×40 mm) broad gauge needle and cell density was measured using a haemocytometer. Cells were further diluted in Growth Medium plus 5% (v/v) charcoal stripped FBS and seeded at a density of 6.5×10³ cells per well (in 90 ul) into transparent, black, tissue culture treated 96 well plates (Packard, No. 6005182).

Test data reported herein was generated using two different compounds preparation and dosing methods. In method (1) a 10 mM compound stock solution in 100% (v/v) DMSO was serially diluted in 4-fold steps in 100% (v/v) DMSO using a Thermo Scientific Matrix SerialMate. The diluted compounds were then further diluted 1 in 30 in assay media using a Thermo Scientific Matrix PlateMate and a 10 μl aliquot of this dilution was dosed to cells manually using a multi-channel pipette. In method (2) starting with a 10 mM compound stock solution, the Labcyte Echo 550 was used to generate a compound concentration response set diluted in 30 μl of assay media. The Echo 550 is a liquid handler that uses acoustic technology to perform direct microplate-to-microplate transfers of DMSO compound solutions. The system can be programmed to transfer volumes as low as 2.5 mL in multiple increments between microplates and in so doing generates a serial dilution of compound which is then back-filled to normalise the DMSO is concentration across the dilution range. A 10 μl volume of diluted compound is then dosed to cells using a Thermo Scientific Matrix PlateMate.

Plates were incubated overnight at 37° C., 5% CO₂. Wells were then dosed with compound prepared by one of the 2 methods above and further incubated for 20-22 hours at 37° C., 5% CO₂. Plates were fixed by the addition of 20 μl of 10% (v/v) formaldehyde solution (in PBS) to each well (final formaldehyde conc.=1.67% (v/v)) and left at room temperature for 10 mins. This fixative solution was removed and cells were washed with 250 μl of PBS/0.05% (v/v) Tween 20 (PBST) using an automated plate washer. This process was then repeated twice more.

Immunostaining was performed at room temperature. Cells were permeabilised by the addition of 35 μl of PBS containing 0.5% Tween 20 and incubated for 1 hour at room temperature. Permeabilisation solution was removed and cells were washed with 250 μl of PBST using an automated plate washer. This process was then repeated twice more. 35 μl of Blocking Solution (PBST containing 3% (w/v) Marvel dried skimmed milk (Nestle)) was added to each well and plates were incubated at room temperature for a minimum of 1 hour. Following removal of the Blocking Solution with a plate washer, 35 μl of mouse anti-human AR monoclonal antibody (clone AR441) (immunogen-synthetic peptide corresponding to amino acids 229-315 of the human AR coupled to keyhole limpet hemocyanin, DAKO, Code No. M3562), diluted 1:500 in Blocking Solution, was added to each well and incubated for 1 hour. Then this primary antibody solution was removed from the wells followed by 3×100 μl PBST washes using a plate washer. Then 35 μl of Alexa-Fluor 488 goat anti-mouse IgG secondary antibody (Invitrogen, Code No. A-11001), diluted 1:500 in Blocking Solution, was added to each well. Henceforth, wherever possible, plates were protected from light exposure. The plates were incubated for 1 hour and then the secondary antibody solution was removed from the wells followed by 3×100 ul PBST washes using a plate washer. Then 50 μl of PBST was added to each well and plates were covered with a black plate seal and stored at 4° C. before being read. Plates were read within six hours of completing the immunostaining

The Green Fluorescent AR-associated signal in each well was measured using an Acumen Explorer HTS Reader (TTP Labtech Ltd., Cambridge). AR-associated fluorescence emission can be detected at 530 nm following excitation at 488 nm. The instrument is a laser-scanning fluorescence microplate cytometer which samples the well at regular intervals and uses threshold algorithms to identify all fluorescent intensities above the solution background without the need to generate and analyse an image. These fluorescent objects can be quantified and provide a measure of the AR levels in cells. Fluorescence dose response data obtained with each compound was exported into a suitable software package (such as Origin) to perform curve fitting analysis. Down-regulation of AR levels was expressed as an IC₅₀ value. This was determined by calculation of the concentration of compound that was required to give a 50% reduction of the AR signal.

The following table discloses biological data for compounds of the present invention using the aforementioned down-regulation assay.

| | Androgen Receptor |
|---|---|
| Example | Down-regulation Assay (a) |
| Number | IC₅₀/μM |
| 1.1 | 1.5 |
| 1.2 | 1.5 |
| 2 | 0.31 |
| 3 | 0.36 |
| 4 | 5.7 |
| 5 | 7.8 |
| 6 | 0.51 |
| 7 | 6.5 |
| 8 | 1 |
| 9 | 0.3 |
| 10 | 0.41 |
| 11 | 1.6 |
| 12 | 0.67 |
| 13 | 0.31 |
| 14 | 0.16 |
| 15 | 0.24 |
| 16 | 0.61 |
| 17 | 0.53 |
| 18 | 0.31 |
| 19 | 0.71 |
| 20 | 0.57 |
| 21 | 1.8 |
| 22 | 1.5 |
| 23 | 0.9 |
| 24 | 1.7 |
| 25 | 1.2 |
| 26 | 0.16 |
| 27 | 0.88 |
| 28 | 0.4 |
| 29 | 0.49 |
| 30 | 0.21 |
| 31 | 0.26 |
| 32 | 0.056 |
| 33 | 0.079 |
| 34 | 0.067 |
| 35 | 0.14 |
| 36 | 0.073 |
| 37 | 0.39 |
| 38 | 0.49 |
| 39 | 0.071 |
| 40 | 0.063 |
| 41 | 0.036 |
| 42 | 0.17 |
| 43 | 0.084 |
| 44 | 0.036 |
| 45 | 0.082 |
| 46 | 0.018 |

### b) Androgen Receptor—Ligand Binding Domain Competitive Binding Assay

The ability of compounds to bind to isolated Androgen Receptor Ligand binding domain (AR-LBD) may be assessed in competition assays using either a Fluorescence Polarisation (FP) or a LanthaScreen™ Time-Resolved Fluorescence Resonance Energy Transfer (TR-FRET) detection end-point.

For the FP test, an assay test kit may be purchased from Invitrogen and used to measure compound binding to the isolated rat AR-LBD which shares 100% sequence identity to the human AR-LBD. The Invitrogen PolarScreen™ Androgen Receptor Competitor Assay Red (Product Code No. PV4293), is a fluorescence polarisation (FP)-based competition assay which measures if test compound can displace a fluorescently-labelled tracer compound. If the test compound binds to the AR-LBD it will prevent the formation of the receptor/tracer complex, which will result in a low polarisation value. If the test compound does not bind the receptor, it will have no effect on formation of the receptor/tracer complex, and the measured polarisation value of the tracer will remain high. The assay is performed as essentially described in the Invitrogen method with the exception that the final assay volume is 12 μl and this requires an appropriate low volume black 384 well microtitre plate. Compounds are dosed directly from a compound source microplate containing serially diluted compound (4 wells containing 10 mM, 0.1 mM, 1 μM and 10 nM final compound respectively) to an assay microplate using an Labcyte Echo 550. The Echo 550 is a liquid handler that uses acoustic technology to perform direct microplate-to-microplate transfers of DMSO compound solutions and the system can be programmed to transfer multiple small nL volumes of compound from the different source plate wells to give the desired serial dilution of compound in the assay which is then back-filled to normalise the DMSO concentration across the dilution range. FP dose response data obtained with each compound is exported into a suitable software package (such as Origin) to perform curve fitting analysis. Competitive AR binding may be expressed as an IC₅₀ value. This is determined by calculation of the concentration of compound that is required to give a 50% reduction in tracer compound binding to AR-LBD.

For LanthaScreen™ TR-FRET, a suitable fluorophore (Product code PV4294) and rat GST-tagged AR-LBD may be purchased from Invitrogen and used to measure compound binding. The assay principle is that AR-LBD is added to a fluorescent ligand to form a receptor/fluorophore complex. A terbium-labelled anti-GST antibody is used to indirectly label the receptor by binding to a GST tag, and competitive binding is detected by a test compounds' ability to displace the fluorescent ligand resulting in a loss of TR-FRET signal between the Tb-anti-GST antibody and the tracer. The assay is performed as follows with all reagent additions carried out using the Thermo Scientific Matrix PlateMate:—
1. Acoustic dispense 120 nl of the test compound into a black low volume 384 well assay plates.
2. Dispense 6 μl of the 2× Fluorophore reagent into each well of the assay plate
3. Dispense 6 μl of the 2×AR-LBD/Tb-anti-GST Ab into each well of the assay plate
4. Cover the assay plate to protect the reagents from light and evaporation, and incubate at room temperature for 1 hour.
5. Excite at 340 nM and measure the fluorescent emission signal of each well at 495 nm and 570 nm using the BMG PheraSTAR.

Compounds may be dosed directly from a compound source microplate containing serially diluted compound (4 wells containing 10 mM, 0.1 mM, 1 μM and 10 nM final compound respectively) to an assay microplate using an Labcyte Echo 550. The Echo 550 is a liquid handler that uses acoustic technology to perform direct microplate-to-microplate transfers of DMSO compound solutions and the system can be programmed to transfer multiple small nL volumes of compound from the different source plate wells to give the desired serial dilution of compound in the assay which is then back-filled to normalise the DMSO concentration across the dilution range. In total 120 mL of compound plus DMSO is added to each well and compounds tested in a 12-point concentration response format over a final compound concentration range of 100, 30, 10, 3, 1, 0.3, 0.1, 0.03, 0.01, 0.003, 0.001, 0.000104, respectively. TR-FRET dose response data obtained with each compound is exported into a suitable software package (such as Origin) to perform curve is fitting analysis. Competitive AR binding may be expressed as an ICₛₒ value. This is determined by calculation of the concentration of compound that is required to give a 50% reduction in tracer compound binding to AR-LBD.

### Pharmaceutical Compositions and Methods of Treatment Comprising Compounds of Formula (I)

According to a further aspect of the invention there is provided a pharmaceutical composition which comprises a compound of Formula (I), or a pharmaceutically acceptable salt thereof, as defined hereinbefore in association with a pharmaceutically-acceptable diluent or carrier. The composition may be in a form suitable for oral administration, for example as a tablet or capsule; for parenteral injection (including intravenous, subcutaneous, intramuscular, intravascular or infusion) as a sterile solution, suspension or emulsion; for topical administration as an ointment or cream; or for rectal administration as a suppository. For example, a composition suitable for intravenous administration comprises 6-(4-{4-[3-(4-acetylpiperazin-1-yl)propoxy]phenyl}piperazin-1-yl)-3-(trifluoromethyl)[1,2,4]triazolo[4,3-b]pyridazine formulated as a solution in 20% w/v HP-β-CD (hydroxypropyl-β-cyclodextrin) in purified water adjusted to pH.4, at concentrations up to 35 mg/mL, corresponding to 65.72 μmol/mL. Alternatively, a composition suitable for oral administration comprises 6-(4-{4-[3-(4-acetylpiperazin-1-yl)propoxy]phenyl}piperazin-1-yl)-3-(trifluoromethyl)[1,2,4]triazolo[4,3-b]pyridazine formulated as a suspension in 0.5% w/v hydroxypropylmethylcellulose (HPMC)/0.1% w/v Tween 80 in purified water at a concentration of 5-50 mg/mL.

For oral administration in a clinical setting, the compound of the invention would preferably be administered in tablet form. For example, the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compound of Formula (I) will normally be administered to a warm-blooded animal at a unit dose within the range 5-5000 mg/m² body area of the animal, i.e. approximately 0.1-100 mg/kg, and this normally provides a therapeutically-effective dose. A unit dose form such as a tablet or capsule will usually contain, for example 1-250 mg of active ingredient. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient. For example, 6-(4-{4-[3-(4-acetylpiperazin-1-yl)propoxy]phenyl}piperazin-1-yl)-3-(trifluoromethyl)[1,2,4]triazolo[4,3-b]pyridazine could be administered to a human patient at a dose of 62 to 320 mg BID (twice a day), more particularly about 220 mg BID, and 1-methyl-4-[2-(4-{1-[3-(trifluoromethyl)[1,2,4]triazolo[4,3-b]pyridazin-6-yl]piperidin-4-yl}phenoxy)ethyl]piperazin-2-one could be administered to a human patient at a dose of 74 to 700 mg BID, more particularly about 250 mg BID. The predicted human doses of 6-(4-{4-[3-(4-acetylpiperazin-1-yl)propoxy]phenyl}piperazin-1-yl)-3-(trifluoromethyl)[1,2,4]triazolo[4,3-b]pyridazine and 1-methyl-4-[2-(4-{1-[3-(trifluoromethyl)[1,2,4]triazolo[4,3-b]pyridazin-6-yl]piperidin-4-yl}phenoxy)ethyl]piperazin-2-one are based on a standard human weighing 70 kg and BID doses are per dose (i.e. half the total daily dose).

For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

We have found that the compounds defined in the present invention are effective modulators of the androgen-receptor. Accordingly, the compounds of the present invention are expected to be potentially useful agents in the treatment of diseases or medical conditions mediated alone or in part by the androgen receptor. Compounds of the invention induce down-regulation of the androgen receptor and/or may be selective agonists, partial agonists, antagonists or partial antagonists of the androgen receptor.

The compounds of the invention may be useful in the treatment of androgen receptor-associated conditions. An “androgen receptor-associated condition,” as used herein, denotes a condition or disorder which can be treated by modulating the function or activity of an androgen receptor in a subject, wherein treatment comprises prevention, partial alleviation or cure of the condition or disorder. Modulation may occur locally, for example, within certain tissues of the subject, or more extensively throughout a subject being treated for such a condition or disorder.

There is therefore provided a compound of Formula (I), or a pharmaceutically acceptable salt thereof, for use as a medicament.

In one embodiment, compounds of the present invention may be administered to animals, for example humans, for the treatment of a variety of conditions and disorders, including, but not limited to the treatment of androgen-sensitive diseases or disorders whose progress or onset is aided by activation of the androgen receptor or androgen receptor modulators. Examples of particular androgen-sensitive diseases or disorders include, but are not limited to, androgen-sensitive cancers such as prostate cancer and other cancers composed of malignant tumor cells containing the androgen receptor, such as is the case for breast, brain, skin, ovarian, bladder, lymphatic, liver and kidney cancers; cancers of the skin, pancreas, endometrium, lung and colon; osteosarcoma; hypercalcemia of malignancy; metastatic bone disease; and androgen senstive disorders such as benign prostatic hyperplasia and prostamegaly, acne (acne vulgaris), seborrhoea, hirsutism (hypertrichosis), androgenic alopecia and male pattern baldness, precocious puberty, endometriosis, polycystic ovarian syndrome, treatment of spermatogenesis, conteracting preeclampsia, eclampsia of pregnancy and preterm labor, treatment of premenstrual syndrome, treatment of vaginal dryness, sexual perversion, virilisation, and the like. Compounds of the invention may also be used to improve ovulation in a domestic animal.

In another embodiment, compounds of the present invention may be administered to animals, for example humans, for the treatment of a variety of conditions and disorders, including, but not limited to maintenance of muscle strength and function (e.g., in the elderly); reversal or prevention of frailty or age-related functional decline (“ARFD”) in the elderly (e.g., sarcopenia); treatment of catabolic side effects of glucocorticoids; prevention and/or treatment of reduced bone mass, density or growth (e.g., osteoporosis and osteopenia); treatment of chronic fatigue syndrome (CFS); chronic myalgia; treatment of acute fatigue syndrome and muscle loss following elective surgery (e.g., post-surgical rehabilitation); accelerating of wound healing; accelerating bone fracture repair (such as accelerating the recovery of hip fracture patients); accelerating healing of complicated fractures, e.g. distraction osteogenesis; in joint replacement; prevention of post-surgical adhesion formation; acceleration of tooth repair or growth; maintenance of sensory function (e.g., hearing, sight, olefaction and taste); treatment of periodontal disease; treatment of wasting secondary to fractures and wasting in connection with chronic obstructive pulmonary disease (COPD), chronic liver disease, AIDS, weightlessness, cancer cachexia, burn and trauma recovery, chronic catabolic state (e.g., coma), eating disorders (e.g., anorexia) and chemotherapy; treatment of cardiomyopathy; treatment of thrombocytopenia; treatment of growth retardation in connection with Crohn's disease; treatment of short bowel syndrome; treatment of irritable bowel syndrome; treatment of inflammatory bowel disease; treatment of Crohn's disease and ulcerative colitis; treatment of complications associated with transplantation; treatment of physiological short stature including growth hormone deficient children and short stature associated with chronic illness; treatment of obesity and growth retardation associated with obesity; treatment of anorexia (e.g., associated with cachexia or aging); treatment of hypercortisolism and Cushing's syndrome; Paget's disease; treatment of osteoarthritis; induction of pulsatile growth hormone release; treatment of osteochondrodysplasias; treatment of depression, nervousness, irritability and stress; treatment of reduced mental energy and low self-esteem (e.g., motivation/assertiveness); improvement of cognitive function (e.g., the treatment of dementia, including Alzheimer's disease and short term memory loss); treatment of catabolism in connection with pulmonary dysfunction and ventilator dependency; treatment of cardiac dysfunction (e.g., associated with valvular disease, myocardial infarction, cardiac hypertrophy or congestive heart failure); lowering blood pressure; protection against ventricular-24 dysfunction or prevention of reperfusion events; treatment of adults in chronic dialysis; reversal or slowing of the catabolic state of aging; attenuation or reversal of protein catabolic responses following trauma (e.g., reversal of the catabolic state associated with surgery, congestive heart failure, cardiac myopathy, burns, cancer, COPD etc.); reducing cachexia and protein loss due to chronic illness such as cancer or AIDS; treatment of hyperinsulinemia including nesidioblastosis; treatment of immunosuppressed patients; treatment of wasting in connection with multiple sclerosis or other neurodegenerative disorders; promotion of myelin repair; maintenance of skin thickness; treatment of metabolic homeostasis and renal homeostasis (e.g., in the frail elderly); stimulation of osteoblasts, bone remodeling and cartilage growth; regulation of food intake; treatment of insulin resistance, including NIDDM, in mammals (e.g., humans); treatment of insulin resistance in the heart; improvement of sleep quality and correction of the relative hyposomatotropism of senescence due to high increase in REM sleep and a decrease in REM latency; treatment of hypothermia; treatment of congestive heart failure; treatment of lipodystrophy (e.g., in patients taking HIV or AIDS therapies such as protease inhibitors); treatment of muscular atrophy (e.g., due to physical inactivity, bed rest or reduced weight-bearing conditions); treatment of musculoskeletal impairment (e.g., in the elderly); improvement of the overall pulmonary function; treatment of sleep disorders; and the treatment of the catabolic state of prolonged critical illness; age related decreased testosterone levels in men, male menopause, hypogonadism, male hormone replacement, male and female sexual dysfunction (e.g., erectile dysfunction, decreased sex drive, sexual well-being, decreased libido), urinary incontinence, male and female contraception, hair loss, and the enhancement of bone and muscle performance/strength.

The term treatment is also intended to include prophylactic treatment.

In addition, the conditions, diseases, and maladies collectively referenced to as “Syndrome X” or Metabolic Syndrome as detailed in Johannsson J. Clin. Endocrinol. Metab., 82, 727-34 (1997), may be treated employing the compounds of the invention.

In one embodiment the androgen-receptor associated conditions include prostate cancer, benign prostatic hyperplasia and prostamegaly, acne (acne vulgaris), seborrhoea, hirsutism (hypertrichosis), androgenic alopecia and male pattern baldness, precocious puberty, polycystic ovarian syndrome, sexual perversion, virilisation, and the like. Compounds of the invention may also be used to improve ovulation in a domestic animal.

Accordingly, the present invention relates to a method of treating any one of the aforementioned androgen-receptor associated condition in a warm-blooded animal, such as man, in need of such treatment which comprises administering to said animal an effective amount of a compound of Formula (I), or a pharmaceutically acceptable salt thereof, as defined hereinbefore.

According to a further aspect the present invention relates to the use of compound of Formula (I), or a pharmaceutically acceptable salt thereof, as hereinbefore defined in the manufacture of a medicament for the treatment of any one of the aforementioned androgen-receptor associated condition.

According to another aspect of the present invention there is provided a compound of Formula (I), or a pharmaceutically acceptable salt thereof, as defined hereinbefore for use in the treatment of any one of the aforementioned androgen-receptor associated condition.

According to another aspect of the present invention there is provided a compound of Formula (I), or a pharmaceutically acceptable salt thereof, as defined hereinbefore for use in a method of treatment of the human or animal body by therapy.

According to a further aspect of the invention there is provided the use of a compound of Formula (I), or a pharmaceutically acceptable salt thereof, as defined hereinbefore in the manufacture of a medicament for use in the production of an anti-androgenic effect in a warm-blooded animal such as man.

According to a further feature of this aspect of the invention there is provided a method for producing an anti-androgenic effect in a warm-blooded animal, such as man, in need of such treatment which comprises administering to said animal an effective amount of a compound of Formula (I), or a pharmaceutically acceptable salt thereof, as defined hereinbefore.

The term “anti-androgenic effect” is used herein to mean the inhibition and/or down regulation of androgen receptors.

According to a further aspect of the invention there is provided the use of a compound of Formula (I), or a pharmaceutically acceptable salt thereof, as defined hereinbefore in the manufacture of a medicament for use in the production of an anti-cell-proliferation effect in a warm-blooded animal such as man.

According to a further feature of this aspect of the invention there is provided a method for producing an anti-cell-proliferation effect in a warm-blooded animal, such as man, in need of such treatment which comprises administering to said animal an effective amount of a compound of Formula (I), or a pharmaceutically acceptable salt thereof, as defined hereinbefore.

According to an additional feature of this aspect of the invention there is provided a method of treating androgen-sensitive cancers in a warm-blooded animal, such as man, in need of such treatment which comprises administering to said animal an effective amount of a compound of Formula (I), or a pharmaceutically acceptable salt thereof, as defined hereinbefore.

According to a further feature of the invention there is provided a compound of Formula (I), or a pharmaceutically acceptable salt thereof, as defined hereinbefore for use in the treatment of androgen-sensitive cancers.

According to a further feature of the invention there is provided the use of a compound of Formula (I), or a pharmaceutically acceptable salt thereof, as defined hereinbefore in the manufacture of a medicament for use in the treatment of androgen-sensitive cancers.

According to an additional feature of this aspect of the invention there is provided a method of treating prostate cancer in a warm-blooded animal, such as man, in need of such treatment which comprises administering to said animal an effective amount of a compound of Formula (I), or a pharmaceutically acceptable salt thereof, as defined hereinbefore. In one embodiment of this aspect of the invention, the prostate cancer is hormone resistant.

According to a further feature of the invention there is provided a compound of Formula (I), or a pharmaceutically acceptable salt thereof, as defined hereinbefore for use in the treatment of prostate cancer, more particularly hormone resistant prostate cancer.

According to a further feature of the invention there is provided the use of a compound of Formula (I), or a pharmaceutically acceptable salt thereof, as defined hereinbefore in the manufacture of a medicament for use in the treatment of prostate cancer, more particularly hormone resistant prostate cancer.

Hormone resistant prostate cancer (HRPC) arises when the prostate cancer progresses to the hormone-independent, castrate resistant stage of the disease.

According to an additional feature of this aspect of the invention there is provided a method of treating any one of the following conditions: benign prostatic hyperplasia, prostamegaly, acne (acne vulgaris), seborrhoea, hirsutism (hypertrichosis), androgenic alopecia and male pattern baldness, precocious puberty, polycystic ovarian syndrome, sexual perversion, or virilisation; in a warm-blooded animal, such as man, in need of such treatment which comprises administering to said animal an effective amount of a compound of Formula (I), or a pharmaceutically acceptable salt thereof, as defined hereinbefore.

According to a further feature of the invention there is provided a compound of Formula (I), or a pharmaceutically acceptable salt thereof, as defined hereinbefore for use in the treatment of any one of the following conditions: benign prostatic hyperplasia, prostamegaly, acne (acne vulgaris), seborrhoea, hirsutism (hypertrichosis), androgenic alopecia and male pattern baldness, precocious puberty, polycystic ovarian syndrome, sexual perversion, or virilisation.

According to a further feature of the invention there is provided the use of a compound of Formula (I), or a pharmaceutically acceptable salt thereof, as defined hereinbefore in the manufacture of a medicament for use in the treatment of any one of the following conditions: benign prostatic hyperplasia, prostamegaly, acne (acne vulgaris), seborrhoea, hirsutism (hypertrichosis), androgenic alopecia and male pattern baldness, precocious puberty, polycystic ovarian syndrome, sexual perversion, or virilisation.

As stated above the size of the dose required for the therapeutic or prophylactic treatment of a particular cell-proliferation disease will necessarily be varied depending on the host treated, the route of administration and the severity of the illness being treated. A unit dose in the range, for example, 1-100 mg/kg, preferably 1-50 mg/kg is envisaged.

The compounds of Formula (I) defined hereinbefore may be applied as a sole therapy or may involve, in addition to the compound of the invention, conventional surgery or radiotherapy or chemotherapy. Such chemotherapy may include one or more of the following categories of anti-tumour agents:—

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan;
(viii) antisense therapies, for example those which are directed to the targets listed above, such as ISIS 2503, an anti-ras antisense;
(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(x) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Thus, according to a further aspect of the invention there is provided a combination suitable for use in the treatment of androgen-sensitive cancers comprising a compound of Formula (I) as defined hereinbefore and or a pharmaceutically acceptable salt thereof etc any one of the anti tumour agents listed under (i)-(x) above. The following deals only with combinations of formula (I)+one agent. Consider if this should be more than one, a class etc.

Therefore, in a further aspect of the invention there is provided a compound of formula (I) or a pharmaceutically acceptable salt thereof etc in combination with an anti-tumour agent selected from one listed under (i)-(x) herein above.

According to a further aspect of the invention there is a provided a combination suitable for use in the treatment of prostrate cancer, in particular HRPC, comprising a compound of Formula (I) or a pharmaceutically acceptable salt thereof, and an agent selected from an androgen-synthesis inhibitor (for example abiraterone); an endothelin receptor antagonist (for example zibotentan (ZD4054) or atrasentan); and an LHRH agonist (for example goserelin, leuprorelin or buserelin).

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided there is provided a kit comprising:

a) a compound of Formula (I) or a pharmaceutically acceptable salt thereof, in a first unit dosage form;

b) an agent selected from an androgen-synthesis inhibitor (for example abiraterone), an endothelin receptor antagonist (for example zibotentan (ZD4054) or atrasentan) and an LHRH agonist (for example goserelin, leuprorelin or buserelin); in a second unit dosage form; and

c) container means for containing said first and second dosage forms.

### EXAMPLES

The invention will now be illustrated in the following Examples in which, generally:

(i) temperatures are given in degrees Celsius (° C.); unless stated otherwise, operations were carried out at room or ambient temperature, that is, at a temperature in the range of 18 to 25° C.;
(ii) organic solutions were dried over anhydrous magnesium sulfate or anhydrous sodium sulfate; evaporation of solvent was carried out using a rotary evaporator under reduced pressure (600 to 4000 Pascals; 4.5 to 30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC and/or analytical LC-MS, and reaction times where given are for illustration only.
(v) final products had satisfactory proton nuclear magnetic resonance (NMR) spectra and/or mass spectral data;
(vi) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 500 MHz using perdeuterio dimethyl sulfoxide (DMSO-d₆) as solvent unless otherwise indicated; the following abbreviations have been used: s, singlet; d, doublet; t, triplet; q, quartet; m, multiplet; bs, broad singlet;
(viii) chemical symbols have their usual meanings; SI units and symbols are used;
(ix) Mass spectra (MS) and LC-MS data were generated on an LC-MS system where the HPLC component comprised generally either an Agilent 1100, Waters Alliance HT (2790 & 2795) equipment or an HP1100 pump and Diode Array with CTC autosampler and was run on a Phenomenex Gemini C18 5 μm, 50×2 mm column (or similar) eluting with either acidic eluent (for example, using a gradient between 0-95% water/acetonitrile with 5% of a 1% formic acid in 50:50 water:acetonitrile (v/v) mixture), or basic eluent (for example, using a gradient between 0-95% water/acetonitrile with 5% of a 0.1% 880 Ammonia in acetonitrile mixture); and the MS component comprised generally a Waters ZQ mass spectrometer scanning over an appropriate mass range. Chromatograms for Electrospray (ESI) positive and negative Base Peak Intensity, and UV Total Absorption Chromatogram from 220-300 nm, are generated and values for m/z are given; generally, only ions which indicate the parent mass are reported and unless otherwise stated the value quoted is the (M+H)+ for positive ion mode and (M−H)− for negative ion mod;
(x) unless stated otherwise compounds containing an asymmetrically substituted carbon and/or sulfur atom have not been resolved;
(xi) any microwave reactions were carried out in either a Biotage Optimizer EXP, or a CEM Explorer microwave;
(xii) preparative high performance liquid chromatography (HPLC) was performed on a Gilson instrument using the following conditions:—
Column: C18 reversed-phase silica, for example, Waters Abridge', 5 μm silica, 19×100 mm, or 30×100 mm, using decreasingly polar solvent mixtures as eluent (decreasing ratio of Solvent A to Solvent B)
Solvent A: Water with 1% ammonium hydroxide

### Solvent B: Acetonitrile

Flow rate: 28 ml/min or 61 ml/min
Gradient: Tailored to suit each compound—generally 7-10 min in length

### Wavelength: 254 nm

(xiii) Strong cation exchange (SCX) chromatography was performed on pre-packed cartridges (for example, ISOLUTE SCX-2 propyl sulfonic acid-based cartridges supplied by International Sorbent Technology), using a basic eluent (for example, 2M ammonia in methanol);
(xiv) The X-ray powder diffraction spectra were determined by mounting a sample of the crystalline material on a Bruker single silicon crystal (SSC) wafer mount and spreading out the sample into a thin layer with the aid of a microscope slide. The sample was spun at 30 revolutions per minute (to improve counting statistics) and irradiated with X-rays generated by a copper long-fine focus tube operated at 40 kV and 40 mA with a wavelength of 1.5406 angstroms. The collimated X-ray source was passed through an automatic variable divergence slit set at V20 and the reflected radiation directed through a 5.89 mm antiscatter slit and a 9.55 mm detector slit. The sample was exposed for 0.03 seconds per 0.00570° 2-theta increment (continuous scan mode) over the range 2 degrees to 40 degrees 2-theta in theta-theta mode. The running time was 3 minutes and 36 seconds. The instrument was equipped with a Position sensitive detector (Lynxeye). Control and data capture was by means of a Dell Optiplex 686 NT 4.0 Workstation operating with Diffract+software.

The % relative intensities of the peaks are categorised in Table 1 below.

TABLE 1

| % Relative Intensity* | Definition |
|---|---|
| 25-100 | vs (very strong) |
| 10-25 | s (strong) |
| 3-10 | m (medium) |
| 1-3 | w (weak) |

*The relative intensities are derived from diffractograms measured with fixed slits
Analytical Instrument: Bruker D4.

(xv) Differential Scanning Calorimetry (DSC) was performed using a TA Instruments Q1000 DSC. Typically less than 5 mg of material contained in a standard aluminium pan fitted with a lid was heated over the temperature range 25° C. to 325° C. at a constant heating rate of 10° C. per minute. A purge gas using nitrogen was used—flow rate 100 ml per minute.
(xvi) the following abbreviations have been used herein, where necessary:—
DCM dichloromethane
DIAD diisopropyl azodicarboxylate
DIPEA N,N-diisopropylethylamine
DMA N,N-dimethyl acetamide
DMF N,N-dimethylformamide
DMSO dimethylsulphoxide
EtOAc ethyl acetate
EtOH ethanol
HATU O-(7-azabenzotriazol-1-yl)-N,N,N′,N′-tetramethyluronium hexafluorophosphate
HPLC high performance liquid chromatography
MeOH methanol
RT room temperature
SCX strong cation exchange
TFA trifluoroacetic acid
THF tetrahydrofuran

### Example 1.1

### Preparation of 6-(4-{4-[3-(4-acetylpiperazin-1-yl)propoxy]phenyl}piperazin-1-yl)-3-(trifluoromethyl)[1,2,4]-triazolo[4,3-b]pyridazine

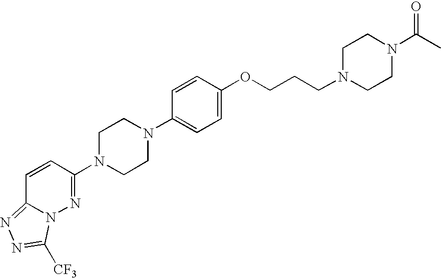

DIPEA (0.160 mL, 0.92 mmol) was added to 6-{4-[4-(3-piperazin-1-ylpropoxy)phenyl]piperazin-1-yl}-3-(trifluoromethyl)[1,2,4]triazolo[4,3-b]pyridazine (150 mg, 0.31 mmol), acetic acid (0.021 mL, 0.37 mmol) and HATU (140 mg, 0.37 mmol) in DMF (2 mL). The resulting solution was stirred at ambient temperature for 16 hours, then purified by preparative HPLC (Waters XBridge Prep C18 OBD column, 5μ silica, 19 mm diameter, 100 mm length), using decreasingly polar mixtures of water (containing 1% ammonia) and MeCN as eluents. Fractions containing the desired compound were evaporated to dryness to give 6-(4-{-4-[3-(4-acetylpiperazin-1-yl)propoxy]phenyl}piperazin-1-yl)-3-(trifluoromethyl)[1,2,4]triazolo[4,3-b]pyridazine (30 mg, 18%).

1H NMR (399.9 MHz, CDCl3) δ 1.96 (2H, m), 2.08 (3H, s), 2.44 (4H, m), 2.54 (2H, t), 3.21 (4H, m), 3.46 (2H, m), 3.62 (2H, m), 3.78 (4H, m), 3.99 (2H, t), 6.87 (2H, d), 6.93 (2H, d), 7.11 (1H, d), 7.96 (1H, d); m/z=533 [M+H]+.

The 6-{4-[4-(3-piperazin-1-ylpropoxy)phenyl]piperazin-1-yl}-3-(trifluoromethyl)[1,2,4]triazolo[4,3-b]pyridazine used as starting material was prepared as follows:—

### Preparation of 4-{4-[3-(trifluoromethyl)[1,2,4]triazolo[4,3-b]pyridazin-6-yl]piperazin-1-yl}phenol

DIPEA (52.4 mL, 300.84 mmol) was added to 6-chloro-3-(trifluoromethyl)-[1,2,4]triazolo[4,3-b]pyridazine (obtained as described in Monatsh. Chem. 1972, 103, 1591) (44.6 g, 200.56 mmol) and 1-(4-hydroxyphenyl)piperazine (39.32 g, 220.61 mmol) in DMF (450 mL). The resulting solution was stirred at 80° C. for 2 hours. The reaction mixture was cooled to room temperature, then evaporated to dryness and partitioned between DCM (2 L) and water (1 L) containing methanol (250 mL) to aid solubility. The insoluble material was collected by filtration, washed with methanol and dried to give the desired product. The organic filtrate was separated from the aqueous, then washed with saturated brine (500 mL), dried over MgSO₄ and evaporated to a brown gum. This was triturated with ether, the resulting solid was collected by filtration, washed with DCM followed by methanol, combined with the previous precipitate and dried to afford 4-{4-[3-(trifluoromethyl)[1,2,4]triazolo[4,3-b]pyridazin-6-yl]piperazin-1-yl}phenol (63.8 g, 87%) as a solid.

1H NMR (399.9 MHz, DMSO-d6) δ 3.12 (4H, m), 3.75 (4H, m), 6.69 (2H, d), 6.87 (2H, d), 7.67 (1H, d), 8.28 (1H, d), 8.87 (1H, s); m/z=365 [M+H]+.

### Preparation of tert-butyl 4-[3-(4-{4-[3-(trifluoromethyl)[1,2,4]triazolo[4,3-b]pyridazin-6-yl]piperazin-1-yl}phenoxy)propyl]piperazine-1-carboxylate

DIAD (3.24 mL, 16.47 mmol) was added dropwise to 4-{4-[3-(trifluoromethyl)[1,2,4]triazolo[4,3-b]pyridazin-6-yl]piperazin-1-yl}phenol (5 g, 13.72 mmol), tert-butyl 4-(3-hydroxypropyl)piperazine-1-carboxylate (CAS 132710-90-8, 5.03 g, 20.59 mmol) and triphenylphosphine (5.40 g, 20.59 mmol) in THF (50 mL) at 0° C. under nitrogen. The resulting solution was stirred at ambient temperature for 16 hours. The reaction mixture was evaporated to dryness and redissolved in DCM (100 mL), and washed sequentially with 2M NaOH (100 mL) and saturated brine (100 mL). The organic layer was dried over MgSO₄, filtered and evaporated to afford crude product. The crude product was purified by flash silica chromatography, eluting with EtOAc. Pure fractions were evaporated to dryness to give tert-butyl 4-[3-(4-{4-[3-(trifluoromethyl)[1,2,4]triazolo[4,3-b]pyridazin-6-yl]piperazin-1-yl}phenoxy)propyl]piperazine-1-carboxylate (3.66 g, 45%).

1H NMR (399.9 MHz, CDCl3) δ 1.46 (9H, s), 1.95 (2H, m), 2.40 (4H, m), 2.52 (2H, t), 3.21 (4H, m), 3.43 (4H, m), 3.78 (4H, m), 3.99 (2H, t), 6.87 (2H, d), 6.93 (2H, d), 7.11 (1H, d), 7.96 (1H, d); m/z=591 [M+H]+.

### Preparation of 6-{4-[4-(3-piperazin-1-ylpropoxy)phenyl]piperazin-1-yl}-3-(trifluoromethyl)[1,2,4]triazolo[4,3-b]pyridazine

TFA (10 mL) was added to tert-butyl 4-[3-(4-{4-[3-(trifluoromethyl)[1,2,4]triazolo[4,3-b]pyridazin-6-yl]piperazin-1-yl}phenoxy)propyl]piperazine-1-carboxylate (1.8 g, 3.05 mmol) in DCM (10 mL). The resulting solution was stirred at ambient temperature for 1 hour then added to an SCX column. The desired product was eluted from the column using 2M ammonia in methanol, the solvents were evaporated and the resulting gum triturated with ether to give 6-{4-[4-(3-piperazin-1-ylpropoxy)phenyl]piperazin-1-yl}-3-(trifluoromethyl)[1,2,4]triazolo[4,3-b]pyridazine (1.380 g, 92%) as a solid.

1H NMR (399.9 MHz, CDCl3) δ 1.95 (2H, m), 2.40-2.52 (6H, m), 2.90 (4H, m), 3.21 (4H, m), 3.78 (4H, m), 3.98 (2H, t), 6.87 (2H, d), 6.93 (2H, d), 7.11 (1H, d), 7.96 (1H, d); m/z=491 [M+H]+.

### Example 1.2

### Large scale preparation of 6-(4-{4-[3-(4-acetylpiperazin-1-yl)propoxy]phenyl}piperazin-1-yl)-3-(trifluoromethyl)[1,2,4]-triazolo[4,3-b]pyridazine

N-Acetylpiperazine (27.3 g, 212.74 mmol) was added to a stirred solution of 3-(4-{1-[3-(trifluoromethyl)[1,2,4]triazolo[4,3-b]pyridazin-6-yl]piperazin-4-yl}phenoxy)propyl methanesulfonate (88.73 g, 177.28 mmol) and DIPEA (35.2 mL, 212.74 mmol) in DMF (444 mL). The mixture was heated to 100° C. for 3.5 hours. A further portion of N-acetylpiperazine (1.136 g, 8.86 mmol) was added and the mixture was heated for a further 60 minutes. The mixture was concentrated to approximately half the volume and ethyl acetate (887 mL) was added. The resultant orange solution was washed with water (887 mL). The combined aqueous phases were further extracted with ethyl acetate (2×887 mL), the aqueous was basified with 2M NaOH to pH8, then extracted with ethyl acetate (2×887 mL). All organic phases were combined and washed with 0.2M NaOH in brine (887 mL), 50% saturated brine (444 mL) and saturated brine (444 mL), dried over MgSO₄, filtered and evaporated to afford 90 g of crude product as a pale yellow solid. The crude product was purified by flash silica chromatography, elution gradient 10 to 30% MeOH in EtOAc. Pure fractions were evaporated to dryness to afford a pale yellow solid, which was purified by recrystallisation from EtOH (799 mL) to afford 6-(4-{4-[3-(4-acetylpiperazin-1-yl)propoxy]phenyl}piperazin-1-yl)-3-(trifluoromethyl)[1,2,4]triazolo[4,3-b]pyridazine (72.5 g, 77%) as a pale yellow crystalline solid.

1H NMR (400.1 MHz, DMSO-d₆) δ 1.82-1.88 (2H, m), 1.98 (3H, s), 2.31 (2H, t), 2.38 (2H, t), 2.43 (2H, t), 3.17 (4H, t), 3.39-3.44 (4H, m), 3.74-3.77 (4H, m), 3.95 (2H, t), 6.84-6.87 (2H, m), 6.93-6.96 (2H, m), 7.66 (1H, d), 8.28 (1H, d); m/z=533 [M+H]+. The 3-(4-{1-[3-(trifluoromethyl)[1,2,4]triazolo[4,3-b]pyridazin-6-yl]piperazin-4-yl}phenoxy)propyl methanesulfonate used as starting material was prepared as follows:—

### Preparation of 4-{4-[3-(trifluoromethyl)[1,2,4]triazolo[4,3-b]pyridazin-6-yl]piperazin-1-yl}phenol

DIPEA (84 mL, 506.17 mmol) and 1-(4-hydroxyphenyl)piperazine (66.2 g, 371.19 mmol) were added to a stirred solution of 6-chloro-3-(trifluoromethyl)[1,2,4]triazolo[4,3-b]pyridazine (obtained as described in Monatsh. Chem. 1972, 103, 1591) (75.1 g, 337.44 mmol) in DMF (751 mL), and the resulting solution was heated to 80° C. for 15 minutes. The mixture was cooled to 20° C., then concentrated to approximately 230 mL. Ethyl acetate (225 mL) and water (1127 mL) were added sequentially, with rapid stirring. A crystalline yellow solid that precipitated out was collected by filtration, washed with water (225 mL, 3 vol), dried on the sinter for 30 minutes, then dried under vacuum at 50° C. overnight to give 4-{4-[3-(trifluoromethyl)[1,2,4]triazolo[4,3-b]pyridazin-6-yl]piperazin-1-yl}phenol (121.2 g, 99%).

1H NMR (399.9 MHz, DMSO-d₆) δ 3.11 (4H, t), 3.74 (4H, t), 6.67-6.71 (2H, m), 6.84-6.88 (2H, m), 7.68 (1H, d), 8.29 (1H, d), 8.91 (1H, s); m/z=365 [M+H]+.

### Preparation of 3-(4-{1-[3-(trifluoromethyl)[1,2,4]triazolo[4,3-b]pyridazin-6-yl]piperazin-4-yl}phenoxy)propan-1-ol

Potassium hydroxide (26.0 g, 464.07 mmol) was added in one portion to a stirred solution of 4-{4-[3-(trifluoromethyl)[1,2,4]triazolo[4,3-b]pyridazin-6-yl]piperazin-1-yl}phenol (113 g, 309.38 mmol) in DMF (564 mL) at ambient temperature. The mixture was heated to 50° C., 3-bromopropan-1-ol (27.0 mL, 309.38 mmol) was added dropwise, and the resultant solution was stirred at 50° C. for 1 hour. The solution was cooled to room temperature, concentrated to approximately 250 mL, then ethyl acetate (225 mL) and water (1127 mL) were added and the biphasic mixture was stirred for 30 minutes. The resulting precipitate was collected by filtration, washed with water (338 mL) and ethyl acetate (225 mL), and dried under vacuum at 50° C. for 4 hours to constant weight to give a pale solid. The solid was stirred in ethyl acetate (564 mL) for 30 minutes, collected by filtration and dried under vacuum at 50° C. for 20 hours to give 3-(4-{1-[3-(trifluoromethyl)[1,2,4]triazolo[4,3-b]pyridazin-6-yl]piperazin-4-yl}phenoxy)propan-1-ol as an off-white solid (109.3 g, 84%).

1H NMR (399.9 MHz, DMSO-d₆) δ 1.80-1.86 (2H, m), 3.16 (4H, t), 3.53-3.57 (2H, m), 3.75 (4H, t), 3.97 (2H, t), 4.54 (1H, s), 6.84-6.87 (2H, m), 6.94-6.97 (2H, m), 7.68 (1H, d), 8.29 (1H, d); m/z=423 [M+H]+.

### Preparation of 3-(4-{1-[3-(trifluoromethyl)[1,2,4]triazolo[4,3-b]pyridazin-6-yl]piperazin-4-yl}phenoxy)propyl methanesulfonate

A solution of methanesulfonyl chloride (24.91 mL, 320.49 mmol) in DCM (433 mL) was added dropwise to a suspension of 3-(4-{1-[3-(trifluoromethyl)[1,2,4]triazolo[4,3-b]pyridazin-6-yl]piperazin-4-yl}phenoxy)propan-1-ol (108.3 g, 256.39 mmol) and triethylamine (50.0 mL, 358.95 mmol) in DCM (1733 mL) at 0° C. under nitrogen. A slight exotherm to 5° C. was observed. The resulting brown solution was stirred for 45 minutes at 0° C. Water (433 mL) was added, the biphasic mixture was stirred for 5 minutes and the phases separated. The organic phase was washed with 50% saturated brine (433 mL), and the brine wash back-extracted with DCM (433 mL). The combined organic phases were dried over MgSO₄, filtered and evaporated to afford 150 g of crude product. The crude product was purified by flash silica chromatography, eluting with EtOAc. Pure fractions were evaporated to dryness to afford 3-(4-{1-[3-(trifluoromethyl)[1,2,4]triazolo[4,3-b]pyridazin-6-yl]piperazin-4-yl}phenoxy)propyl methanesulfonate (97 g, 76%) as a pale yellow solid.

1H NMR (399.9 MHz, CDCl3) δ 2.19-2.25 (2H, m), 3.00 (3H, s), 3.22 (4H, t), 3.79 (4H, t), 4.06 (2H, t), 4.45 (2H, t), 6.86-6.89 (2H, m), 6.92-6.95 (2H, m), 7.13 (1H, d), 7.97 (1H, d); m/z=501 [M+H]+.

### Example 1.3

### Preparation of 6-(4-{4-[3-(4-acetylpiperazin-1-yl)propoxy]phenyl}piperazin-1-yl)-3-(trifluoromethyl)[1,2,4]triazolo[4,3-b]pyridazine Form A

The X-ray powder diffraction spectra for 6-(4-{4-[3-(4-acetylpiperazin-1-yl)propoxy]phenyl}piperazin-1-yl)-3-(trifluoromethyl)[1,2,4]triazolo[4,3-b]pyridazine showed the material to be crystalline. This material had a melting point of 162.02° C. (onset). Organic slurries of crystalline 6-(4-{4-[3-(4-acetylpiperazin-1-yl)propoxy]phenyl}piperazin-1-yl)-3-(trifluoromethyl)[1,2,4]triazolo[4,3-b]pyridazine produced Form A. Slurries were conducted by measuring approximately 20 mg of the original material into a vial with a magnetic flea, and approximately 2 ml of methanol, acetonitrile, aqueous methanol or ethylacetate was added, the vial was then sealed tightly with a cap and left to stir on a magnetic stirrer plate. After 3 days, the sample was removed from the plate, the cap taken off and the slurry left to dry under ambient conditions before it was analysed by XRPD and DSC. All material produced in these slurries remained as Form A.

6-(4-{4-[3-(4-acetylpiperazin-1-yl)propoxy]phenyl}piperazin-1-yl)-3-(trifluoromethyl)[1,2,4]triazolo[4,3-b]pyridazine Form A is characterised by providing at least one of the following 2θ values measured using CuKa radiation: 17.0 and 8.0° and by providing an X-ray powder diffraction pattern, substantially as shown in Figure A. The ten most prominent peaks are shown in Table A:

### Table A

### Ten most Prominent X-Ray Powder Diffraction peaks for 6-(4-{4-[3-(4-acetylpiperazin-1-yl)propoxy]phenyl}piperazin-1-yl)-3-(trifluoromethyl)[1,2,4]triazolo[4,3-b]pyridazine

### Form A

| Angle 2- | | Relative |
|---|---|---|
| Theta (2θ) | Intensity % | Intensity |
| 16.984 | 100.0 | vs |
| 7.982 | 41.5 | vs |
| 18.004 | 38.6 | vs |
| 22.017 | 31.2 | vs |
| 17.757 | 30.7 | vs |
| 21.006 | 28.7 | vs |
| 10.758 | 27.3 | vs |
| 8.515 | 22.4 | s |
| 21.657 | 22.4 | s |
| 18.489 | 19.3 | s |

vs = very strong
s = strong

DSC analysis of 6-(4-{4-[3-(4-acetylpiperazin-1-yl)propoxy]phenyl}piperazin-1-yl)-3-(trifluoromethyl)[1,2,4]triazolo[4,3-b]pyridazine Form A showed a single thermal event with an onset at 162.02° C. and a peak at 163.92° C. corresponding to the melt of Form A (Figure B). DSC analysis thus showed that 6-(4-{4-[3-(4-acetylpiperazin-1-yl)propoxy]phenyl}piperazin-1-yl)-3-(trifluoromethyl)[1,2,4]triazolo[4,3-b]pyridazine Form A is a moderate melting solid with an onset of melting at about 162.02° C. and a peak at about 163.92° C.

### Example 2

### Preparation of 1-methyl-4-[2-(4-{1-[3-(trifluoromethyl)[1,2,4]-triazolo[4,3-b]pyridazin-6-yl]piperidin-4-yl}phenoxy)ethyl]piperazin-2-one

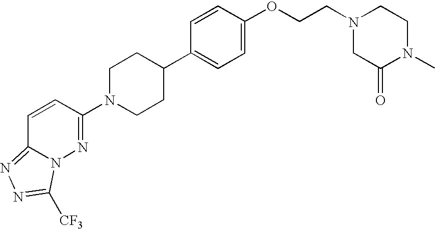

DIPEA (15.27 mL, 87.69 mmol) was added to 2-(4-{1-[3-(trifluoromethyl)[1,2,4]triazolo[4,3-b]pyridazin-6-yl]piperidin-4-yl}phenoxy)ethyl methanesulfonate (14.19 g, 29.23 mmol) and 1-methylpiperazin-2-one (CAS 59702-07-7, 3.67 g, 32.15 mmol) in DMA (70 mL). The resulting solution was stirred at 110° C. for 1 hour. The reaction mixture was cooled to room temperature, absorbed onto silica, evaporated to dryness and then purified by flash silica chromatography, elution gradient 0 to 3% MeOH in DCM. Pure fractions were evaporated and the resulting gum was scratched with ether until solid. The solid was stirred in ether (100 mL) for 4 hours then collected by filtration and dried to give 1-methyl-4-[2-(4-{1-[3-(trifluoromethyl)[1,2,4]triazolo[4,3-b]pyridazin-6-yl]piperidin-4-yl}phenoxy)ethyl]piperazin-2-one (10.08 g, 68.5%) as a solid.

1H NMR (399.9 MHz, CDCl3) δ 1.76 (2H, m), 2.00 (2H, m), 2.75-2.87 (5H, m), 2.95 (3H, s), 3.11 (2H, m), 3.28 (2H, s), 3.34 (2H, t), 4.09 (2H, t), 4.37 (2H, m), 6.86 (2H, d), 7.11-7.14 (3H, m), 7.92 (1H, d); m/z=504 [M+H]+.

The 2-(4-{1-[3-(trifluoromethyl)[1,2,4]triazolo[4,3-b]pyridazin-6-yl]piperidin-4-yl}phenoxy)ethyl methanesulfonate used as starting material was prepared as follows:—

### Preparation of benzyl 4-(trifluoromethylsulfonyloxy)-5,6-dihydropyridine-1(2H)-carboxylate

A solution of benzyl 4-oxopiperidine-1-carboxylate (88.57 g, 379.70 mmol) in THF (300 mL) was added dropwise to lithium bis(trimethylsilyl)amide (1M in THF) (418 mL, 417.67 mmol) at −78° C., over a period of 1 hour under nitrogen. The resulting mixture was stirred at −78° C. for 90 minutes then a solution of 1,1,1-trifluoro-N-phenyl-N-(trifluoromethylsulfonyl)methanesulfonamide (142 g, 398.68 mmol) in THF (600 mL) was added dropwise over a period of 1 hour. The resulting mixture was stirred at −78° C. for 30 minutes, then allowed to warm to room temperature and stirred for 16 hours. The reaction mixture was quenched with 2M NaOH (450 mL). The layers were separated and the organic layer was washed with 2M NaOH (360 mL). The solvent was evaporated, then the residue was re-dissolved in diethyl ether (1500 mL) and the solution washed with water (500 mL). The organic layer was dried over MgSO4, filtered and evaporated to afford benzyl 4-(trifluoromethylsulfonyloxy)-5,6-dihydropyridine-1(2H)-carboxylate (124 g, 81%) as an oil.

1H NMR (399.9 MHz, DMSO-d6) δ 2.43 (2H, m), 3.62 (2H, m), 4.06 (2H, m), 5.10 (2H, s), 6.02 (1H, m), 7.34 (5H, m).

### Preparation of benzyl 4-(4-hydroxyphenyl)-5,6-dihydropyridine-1(2H)-carboxylate

Sodium carbonate (96 g, 909.79 mmol) was added to benzyl 4-(trifluoromethylsulfonyloxy)-5,6-dihydropyridine-1(2H)-carboxylate (123.1 g, 303.26 mmol) and 4-hydroxyphenylboronic acid (46.0 g, 333.59 mmol) in a mixture of dioxane (1000 mL) and water (250 mL). The resulting mixture was bubbled with nitrogen for 10 minutes then 1,1′-bis(diphenylphosphino)ferrocenedichloropalladium(II) (5.49 g, 7.58 mmol) was added and the reaction mixture was heated at 80° C. for 1 hour, then cooled to room temperature. The reaction mixture was diluted with DCM (2 L) and washed with water (2 L). The aqueous washing was re-extracted with DCM (1 L), then the combined organics were washed with saturated brine (500 mL), dried over MgSO4, filtered and evaporated to afford crude product. The crude product was purified by flash silica chromatography, elution gradient 10 to 30% EtOAc in isohexane. Fractions containing the desired product were evaporated to dryness then triturated with isohexane, filtered and dried to afford benzyl 4-(4-hydroxyphenyl)-5,6-dihydropyridine-1(2H)-carboxylate (62.3 g, 66.4%) as a solid.

1H NMR (399.9 MHz, DMSO-d6) δ 2.44 (2H, m), 3.61 (2H, m), 4.05 (2H, m), 5.12 (2H, s), 5.99 (1H, m), 6.73 (2H, d), 7.26 (2H, d), 7.32-7.40 (5H, m), 9.45 (1H, s); m/z=310 [M+H]+.

### Preparation of 4-(piperidin-4-yl)phenol

Benzyl 4-(4-hydroxyphenyl)-5,6-dihydropyridine-1(2H)-carboxylate (37.7 g, 121.86 mmol) and 5% palladium on carbon (7.6 g, 3.57 mmol) in methanol (380 mL) were stirred under an atmosphere of hydrogen at 5 bar and 25° C. for 2 hours. The catalyst was removed by filtration, washed with MeOH and the solvents evaporated. The crude material was triturated with diethyl ether, then the desired product collected by filtration and dried under vacuum to afford 4-(piperidin-4-yl)phenol (20.36 g, 94%) as a solid.

1H NMR (399.9 MHz, DMSO-d6) δ 1.46 (2H, m), 1.65 (2H, m), 2.45 (1H, m), 2.58 (2H, m), 3.02 (2H, m), 6.68 (2H, d), 7.00 (2H, d), 9.15 (1H, s); m/z=178 [M+H]+.

### Preparation of 4-{1-[3-(trifluoromethyl)[1,2,4]triazolo[4,3-b]pyridazin-6-yl]piperidin-4-yl}phenol

DIPEA (48.2 mL, 276.86 mmol) was added to 6-chloro-3-(trifluoromethyl)-[1,2,4]triazolo[4,3-b]pyridazine (obtained as described in Monatsh. Chem. 1972, 103, 1591) (24.65 g, 110.74 mmol) and 4-(piperidin-4-yl)phenol (20.61 g, 116.28 mmol) in DMF (200 mL). The resulting solution was stirred at 80° C. for 1 hour. The reaction mixture was cooled to room temperature, then evaporated to dryness and re-dissolved in DCM (1 L) and washed with water (2×1 L). The organic layer was washed with saturated brine (500 mL), then dried over MgSO4, filtered and evaporated to afford crude product. The crude product was triturated with ether to afford 4-{1-[3-(trifluoromethyl)[1,2,4]triazolo[4,3-b]pyridazin-6-yl]piperidin-4-yl}phenol (36.6 g, 91%) as a solid.

1H NMR (399.9 MHz, DMSO-d6) δ 1.64 (2H, m), 1.87 (2H, m), 2.75 (1H, m), 3.09 (2H, m), 4.40 (2H, m), 6.69 (2H, d), 7.05 (2H, d), 7.65 (1H, d), 8.24 (1H, d), 9.15 (1H, s); m/z=364 [M+H]+.

### Preparation of 2-(4-{1-[3-(trifluoromethyl)[1,2,4]triazolo[4,3-b]pyridazin-6-yl]piperidin-4-yl}phenoxy)ethanol

A solution of ethylene carbonate (18.18 g, 206.42 mmol) in DMF (30 mL) was added dropwise to a stirred suspension of 4-{1-[3-(trifluoromethyl)[1,2,4]triazolo[4,3-b]pyridazin-6-yl]piperidin-4-yl}phenol (15 g, 41.28 mmol) and potassium carbonate (11.41 g, 82.57 mmol) in DMF (30 mL) at 80° C. over a period of 10 minutes under nitrogen. The resulting mixture was stirred at 80° C. for 20 hours. The reaction mixture was cooled to room temperature then concentrated and diluted with DCM (500 mL), and washed sequentially with water (500 mL) and saturated brine (250 mL). The organic layer was dried over MgSO4, filtered and evaporated to afford crude product. The crude product was purified by flash silica chromatography, elution gradient 70 to 100% EtOAc in isohexane. Pure fractions were evaporated to dryness to give 2-(4-{1-[3-(trifluoromethyl)[1,2,4]triazolo[4,3-b]pyridazin-6-yl]piperidin-4-yl}phenoxy)ethanol (12.04 g, 71.6%) as a solid.

1H NMR (399.9 MHz, DMSO-d6) δ 1.67 (2H, m), 1.89 (2H, m), 2.81 (1H, m), 3.10 (2H, m), 3.70 (2H, m), 3.95 (2H, t), 4.41 (2H, m), 4.81 (1H, t), 6.87 (2H, d), 7.18 (2H, d), 7.66 (1H, d), 8.24 (1H, d); m/z=408 [M+H]+.

### Preparation of 2-(4-{1-[3-(trifluoromethyl)[1,2,4]triazolo[4,3-b]pyridazin-6-yl]piperidin-4-yl}phenoxy)ethyl methanesulfonate

A solution of methanesulfonyl chloride (2.74 mL, 35.46 mmol) in DCM (40 mL) was added dropwise to a stirred solution of 2-(4-{1-[3-(trifluoromethyl)[1,2,4]triazolo[4,3-b]pyridazin-6-yl]piperidin-4-yl}phenoxy)ethanol (12.04 g, 29.55 mmol) and triethylamine (8.24 mL, 59.11 mmol) in DCM (120 mL) cooled to 0° C. under nitrogen. The resulting solution was stirred at 0° C. for 15 minutes then warmed to room temperature and stirred for a further 15 minutes. The reaction mixture was diluted with DCM (100 mL), and washed with water (250 mL) and saturated brine (100 mL). The organic layer was dried over MgSO4, filtered and evaporated to give 2-(4-{1-[3-(trifluoromethyl)[1,2,4]triazolo[4,3-b]pyridazin-6-yl]piperidin-4-yl}phenoxy)ethyl methanesulfonate (14.32 g, 100%) as a solid.

1H NMR (399.9 MHz, CDCl3) δ 1.70 (2H, m), 1.93 (2H, m), 2.73 (1H, m), 3.00-3.08 (5H, m), 4.17 (2H, m), 4.30 (2H, m), 4.49 (2H, m), 6.80 (2H, d), 7.04-7.10 (3H, m), 7.86 (1H, d); m/z=486 [M+H]+.

### Example 3

### Preparation of 4-[2-(4-[4-hydroxy-1-[3-(trifluoromethyl)[1,2,4]-triazolo[4,3-b]pyridazin-6-yl]piperidin-4-yl]phenoxy)ethyl]-1-methylpiperazin-2-one

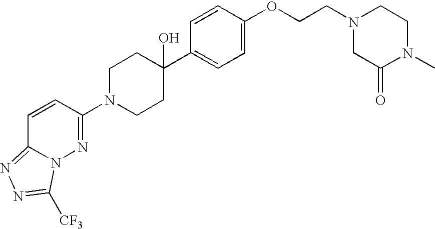

DIAD (0.234 mL, 1.19 mmol) was added dropwise to 4-(4-hydroxyphenyl)-1-[3-(trifluoromethyl)[1,2,4]triazolo[4,3-b]pyridazin-6-yl]piperidin-4-ol (300 mg, 0.79 mmol), 4-(2-hydroxyethyl)-1-methylpiperazin-2-one (188 mg, 1.19 mmol) and triphenylphosphine (311 mg, 1.19 mmol) in THF (3 mL) under nitrogen. The resulting solution was stirred at ambient temperature for 16 hours then the solvent was evaporated. The crude product was purified by flash silica chromatography, elution gradient 80 to 100% EtOAc in isohexane then 0 to 5% MeOH in DCM. Pure fractions were evaporated to dryness to give a gum which was scratched with ether. The resulting solid was collected by filtration and dried to give 4-(2-(4-(4-hydroxy-1-(3-(trifluoromethyl)-[1,2,4]triazolo[4,3-b]pyridazin-6-yl)piperidin-4-yl)phenoxy)ethyl)-1-methylpiperazin-2-one (185 mg, 45%) as a solid.

1H NMR (399.9 MHz, CDCl3) δ 1.87 (2H, m), 2.06 (2H, m), 2.79 (4H, m), 2.88 (3H, s), 3.21 (2H, s), 3.27 (2H, t), 3.52 (2H, m), 4.03-4.09 (4H, m), 6.84 (2H, d), 7.06 (1H, d), 7.33 (2H, d), 7.85 (1H, d); m/z=520 [M+H]+.

The 4-(2-hydroxyethyl)-1-methylpiperazin-2-one used as starting material was prepared as follows:—

2-Bromoethanol (5.60 mL, 78.85 mmol) was added to 1-methylpiperazin-2-one (1.80 g, 15.77 mmol) and potassium carbonate (6.54 g, 47.31 mmol) in THF (20 mL). The resulting mixture was stirred at 65° C. for 16 hours. The mixture was cooled to room temperature, filtered and the solvents evaporated to give crude product. The crude product was purified by flash silica chromatography, elution gradient 0 to 8% MeOH in DCM. Pure fractions were evaporated to dryness to give 4-(2-hydroxyethyl)-1-methylpiperazin-2-one (1.870 g, 75.0%).

1H NMR (399.9 MHz, CDCl3) δ 2.55 (2H, t), 2.71 (2H, t), 2.90 (3H, s), 3.15 (2H, s), 3.28 (2H, t), 3.60 (2H, t)

The 4-(4-hydroxyphenyl)-1-[3-(trifluoromethyl)[1,2,4]triazolo[4,3-b]pyridazin-6-yl]piperidin-4-ol used as starting material was prepared as follows:—

### Preparation of benzyl 4-[4-(benzyloxy)phenyl]-4-hydroxypiperidine-1-carboxylate

n-Butyllithium (1.6M in hexane, 42.9 ml, 107.18 mmol) was added dropwise to 1-(benzyloxy)-4-bromobenzene (28.2 g, 107.18 mmol, CAS 6793-92-6) in THF (367 ml) at −78° C. over a period of 15 minutes under nitrogen. The resulting solution was stirred at −78° C. for 1 hour then benzyl 4-oxopiperidine-1-carboxylate (20 g, 85.74 mmol) in THF (122 ml) was added dropwise. The resulting mixture was stirred at −78° C. for 10 minutes, then allowed to warm to room temperature and stirred for 16 hours. The reaction mixture was evaporated to dryness and quenched with saturated ammonium chloride (50 mL), then then extracted with EtOAc (500 mL). The organic layer was dried over MgSO4, filtered and evaporated to afford crude product. The crude product was purified by flash silica chromatography, elution gradient 1 to 100% EtOAc in isohexane. Pure fractions were evaporated to dryness to afford crude product. The crude product was further purified by flash silica chromatography, elution gradient 0 to 3% MeOH in DCM. Pure fractions were evaporated to dryness to afford benzyl 4-[4-(benzyloxy)phenyl]-4-hydroxypiperidine-1-carboxylate (13.49 g, 30.1%) as a gum.

1H NMR (399.9 MHz, DMSO-d6) δ 1.58 (2H, m), 1.80 (2H, m), 3.27 (2H, m), 3.71 (1H, m), 3.92 (2H, m), 5.10 (4H, m), 6.95 (2H, m), 7.39 (12H, m); m/z=416 [M−H]+.

### Preparation of 4-(4-hydroxyphenyl)piperidin-4-ol

10% Palladium on carbon (3.44 g, 3.23 mmol) was added to benzyl 4-[4-(benzyloxy)phenyl]-4-hydroxypiperidine-1-carboxylate (13.49 g, 32.31 mmol) in MeOH (146 mL). The resulting mixture was stirred at room temperature for 20 hours under an atmosphere of hydrogen. The reaction mixture was filtered and evaporated to afford crude product. The crude material was triturated with DCM (100 mL) and MeOH (50 mL) to give a solid which was collected by filtration and dried under vacuum to give 4-(4-hydroxyphenyl)piperidin-4-ol (4.16 g, 66.6%).

1H NMR (399.9 MHz, DMSO-d6) δ 1.50 (2H, m), 1.73 (2H, m), 2.70 (2H, m), 2.90 (2H, m), 4.52 (1H, s), 6.69 (2H, m), 7.25 (2H, m), 9.21 (1H, s); m/z=192 [M−H]+.

### Preparation of 4-(4-hydroxyphenyl)-1-[3-(trifluoromethyl)[1,2,4]triazolo[4,3-b]pyridazin-6-yl]piperidin-4-ol

DIPEA (1.174 mL, 6.74 mmol) was added to 6-chloro-3-(trifluoromethyl)-[1,2,4]triazolo[4,3-b]pyridazine (obtained as described in Monatsh. Chem. 1972, 103, 1591) (1 g, 4.49 mmol) and 4-(4-hydroxyphenyl)piperidin-4-ol (0.955 g, 4.94 mmol) in DMF (10 mL). The resulting solution was stirred at 80° C. for 1 hour. The reaction mixture was cooled to room temperature then evaporated to dryness. The residues were triturated with water and the resulting solid collected by filtration, washed with further water followed by ether, then dried under vacuum to afford 4-(4-hydroxyphenyl)-1-[3-(trifluoromethyl)[1,2,4]triazolo[4,3-b]pyridazin-6-yl]piperidin-4-ol (1.680 g, 99%) as a solid.

1H NMR (399.9 MHz, DMSO-d6) δ 1.72 (2H, m), 1.95 (2H, m), 3.42 (2H, m), 4.17 (2H, m), 5.01 (1H, s), 6.70 (2H, d), 7.28 (2H, d), 7.65 (1H, d), 8.23 (1H, d), 9.20 (1H, s); m/z=380 [M+H]+.

### Example 4

### Preparation of 1-methyl-4-[2-(4-{4-[3-(trifluoromethyl)[1,2,4]triazolo[4,3-b]pyridazin-6-yl]piperazin-1-yl}phenoxy)ethyl]piperazin-2-one

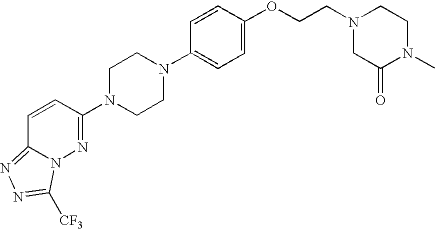

2-(4-{4-[3-(Trifluoromethyl)[1,2,4]triazolo[4,3-b]pyridazin-6-yl]piperazin-1-yl}phenoxy)ethyl methanesulfonate (300 mg, 0.62 mmol), 1-methylpiperazin-2-one (339 mg, 0.93 mmol), DIPEA (0.644 mL, 3.70 mmol) and sodium iodide (9.24 mg, 0.06 mmol) were suspended in DMA (3 mL) and sealed into a microwave tube. The reaction was heated to 150° C. for 1 hour in the microwave reactor and cooled to room temperature. The reaction mixture was absorbed on to silica, evaporated and purified by flash silica chromatography, elution gradient 0 to 5% MeOH in DCM. Pure fractions were evaporated to dryness to afford a gum, which was further purified by ion exchange chromatography using an SCX column, eluting from the column with 2M ammonia/MeOH. Further purification by flash silica chromatography, elution gradient 0 to 5% MeOH in DCM gave 1-methyl-4-[2-(4-{4-[3-(trifluoromethyl)[1,2,4]triazolo[4,3-b]pyridazin-6-yl]piperazin-1-yl}phenoxy)ethyl]piperazin-2-one (44.0 mg, 14.14%) as a solid.

1H NMR (399.9 MHz, CDCl3) δ 2.78 (4H, m), 2.89 (3H, s), 3.15 (4H, m), 3.22 (2H, s), 3.28 (2H, t), 3.71 (4H, m), 4.01 (2H, t), 6.80 (2H, d), 6.86 (2H, d), 7.05 (1H, d), 7.89 (1H, d); m/z=505 [M+H]+.

The 2-(4-{4-[3-(trifluoromethyl)[1,2,4]triazolo[4,3-b]pyridazin-6-yl]piperazin-1-yl}phenoxy)ethyl methanesulfonate used as starting material was prepared as follows:—

### Preparation of 2-(4-{4-[3-(trifluoromethyl)[1,2,4]triazolo[4,3-b]pyridazin-6-yl]piperazin-1-yl}phenoxy)ethanol

Obtained in 51% yield by an analogous method to Example 2, preparation of starting materials, starting from 4-{4-[3-(trifluoromethyl)[1,2,4]triazolo[4,3-b]pyridazin-6-yl]piperazin-1-yl}phenol (obtained as described in Example 1.2, preparation of starting materials).

1H NMR (399.9 MHz, DMSO-d6) δ 3.18 (4H, m), 3.69 (2H, m), 3.76 (4H, m), 3.93 (2H, t), 4.80 (1H, t), 6.87 (2H, d), 6.96 (2H, d), 7.67 (1H, d), 8.29 (1H, d); m/z=409 [M+H]+.

### Preparation of 2-(4-{4-[3-(trifluoromethyl)[1,2,4]triazolo[4,3-b]pyridazin-6-yl]piperazin-1-yl}phenoxy)ethyl methanesulfonate

A solution of methanesulfonyl chloride (0.195 mL, 2.52 mmol) in DCM (5 mL) was added dropwise to a stirred suspension of 2-(4-{4-[3-(trifluoromethyl)[1,2,4]triazolo[4,3-b]pyridazin-6-yl]piperazin-1-yl}phenoxy)ethanol (856 mg, 2.10 mmol) and triethylamine (0.584 mL, 4.19 mmol) in DCM (15 mL) cooled to 0° C. under nitrogen. The resulting solution was stirred at 0° C. for 15 minutes then warmed to room temperature and stirred for a further 15 minutes. The reaction mixture was diluted with DCM (20 mL), and washed with water (25 mL) and saturated brine (25 mL). The organic layer was dried over MgSO4, filtered and evaporated to afford crude product. The crude product was purified by flash silica chromatography, elution gradient 0 to 3% MeOH in DCM. Pure fractions were evaporated to dryness to give 2-(4-{4-[3-(trifluoromethyl)[1,2,4]triazolo[4,3-b]pyridazin-6-yl]piperazin-1-yl}phenoxy)ethyl methanesulfonate (737 mg, 72%).

1H NMR (399.9 MHz, DMSO-d6) δ 3.20 (4H, m), 3.23 (3H, s), 3.76 (4H, m), 4.20 (2H, m), 4.51 (2H, m), 6.91 (2H, d), 6.99 (2H, d), 7.68 (1H, d), 8.29 (1H, d); m/z=487 [M+H]+.

### Example 5

### Preparation of 1-cyclopropyl-4-[2-(4-{4-[3-(trifluoromethyl)[1,2,4]-triazolo[4,3-b]pyridazin-6-yl]piperazin-1-yl}phenoxy)ethyl]piperazin-2-one

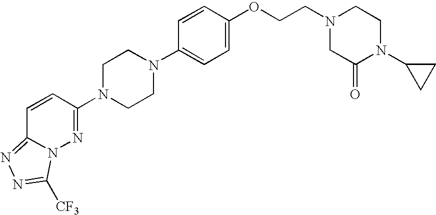

1-Cyclopropylpiperazin-2-one (144 mg, 1.03 mmol) was added to 2-(4-{4-[3-(trifluoromethyl)[1,2,4]triazolo[4,3-b]pyridazin-6-yl]piperazin-1-yl}phenoxy)ethyl methanesulfonate (obtained as described in Example 4, preparation of starting materials) (100 mg, 0.21 mmol) and DIPEA (0.072 mL, 0.41 mmol) in THF (2 mL). The resulting mixture was stirred at 65° C. for 16 hours then NMP (0.5 mL) was added and stirring continued at 65° C. for a further 8 hours. The solvents were evaporated to give crude product, which was purified by preparative HPLC (Waters XBridge Prep C18 OBD column, 5μ silica, 19 mm diameter, 100 mm length), using decreasingly polar mixtures of water (containing 1% ammonia) and MeCN as eluents. Fractions containing the desired compound were evaporated to dryness to give 1-cyclopropyl-4-[2-(4-{-4-[3-(trifluoromethyl)[1,2,4]triazolo[4,3-b]pyridazin-6-yl]piperazin-1-yl}phenoxy)ethyl]piperazin-2-one (22 mg, 20%) as a solid.

1H NMR (399.9 MHz, CDCl3) δ 0.67 (2H, m), 0.81 (2H, m), 2.72 (1H, m), 2.81 (4H, m), 3.22 (4H, m), 3.27-3.31 (4H, m), 3.78 (4H, m), 4.07 (2H, m), 6.86-6.94 (4H, m), 7.11 (1H, d), 7.96 (1H, d); m/z=531 [M+H]+.

### Example 6

### Preparation of 1-cyclopropyl-4-[2-(4-{1-[3-(trifluoromethyl)[1,2,4]-triazolo[4,3-b]pyridazin-6-yl]piperidin-4-yl}phenoxy)ethyl]piperazin-2-one

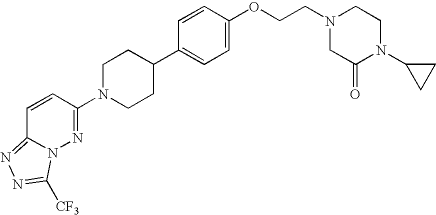

2-(4-{1-[3-(Trifluoromethyl)[1,2,4]triazolo[4,3-b]pyridazin-6-yl]piperidin-4-yl}phenoxy)ethyl methanesulfonate (obtained as described in Example 2, preparation of starting materials) (180 mg, 0.37 mmol), 1-cyclopropylpiperazin-2-one (62.4 mg, 0.44 mmol) and DIPEA (0.194 mL, 1.11 mmol) were dissolved in DMA (2 mL) and sealed into a microwave tube. The reaction was heated to 150° C. for 1 hour in the microwave reactor and cooled to room temperature. The reaction was incomplete and further 1-cyclopropylpiperazin-2-one (62.4 mg, 0.44 mmol) was added, and the solution was stirred at 150° C. for a further 1 hour, then cooled to room temperature. The crude reaction mixture was absorbed onto silica and purified by flash silica chromatography, elution gradient 0 to 3% MeOH in DCM. Pure fractions were evaporated to dryness to afford a gum, which was triturated with ether. The resulting solid was collected by filtration and dried to give 1-1-cyclopropyl-4-[2-(4-{1-[3-(trifluoromethyl)[1,2,4]triazolo[4,3-b]pyridazin-6-yl]piperidin-4-yl}phenoxy)ethyl]piperazin-2-one (73 mg, 37%) as a solid.

1H NMR (399.9 MHz, CDCl3) δ 0.59 (2H, m), 0.74 (2H, m), 1.69 (2H, m), 1.93 (2H, m), 2.61-2.78 (6H, m), 3.04 (2H, m), 3.21 (4H, m), 4.01 (2H, t), 4.30 (2H, m), 6.79 (2H, d), 7.06 (3H, m), 7.85 (1H, d); m/z=530 [M+H]+.

### Example 7

### Preparation of 4-methyl-1-[2-(4-{4-[3-(trifluoromethyl)[1,2,4]-triazolo[4,3-b]pyridazin-6-yl]piperazin-1-yl}phenoxy)ethyl]piperazin-2-one

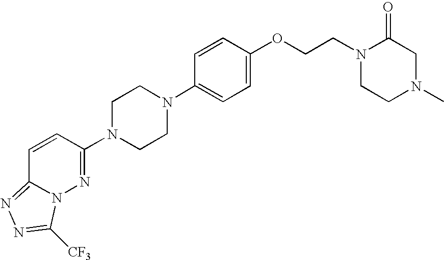

Formaldehyde (37% aqueous solution) (1 mL, 0.09 mmol) was added to 1-[2-(4-{4-[3-(trifluoromethyl)[1,2,4]triazolo[4,3-b]pyridazin-6-yl]piperazin-1-yl}phenoxy)ethyl]piperazin-2-one (44 mg, 0.09 mmol) and acetic acid (5 μL, 0.09 mmol) in a mixture of THF (5 mL), DCM (5 mL) and methanol (1 mL). The resulting mixture was stirred at ambient temperature for 30 minutes then sodium triacetoxyhydroborate (57 mg, 0.27 mmol) was added and the mixture stirred for a further 30 minutes. The solvents were evaporated and the residues neutralised with saturated aqueous NaHCO₃, diluted with water (5 mL) and purified by preparative HPLC (Waters XBridge Prep C18 OBD column, 5μ silica, 19 mm diameter, 100 mm length), using decreasingly polar mixtures of water (containing 1% ammonia) and MeCN as eluents. Fractions containing the desired compound were evaporated to dryness to give 4-methyl-1-[2-(4-{4-[3-(trifluoromethyl)[1,2,4]triazolo[4,3-b]pyridazin-6-yl]piperazin-1-yl}phenoxy)ethyl]piperazin-2-one (23 mg, 50%) as a solid.

1H NMR (399.9 MHz, CDCl3) δ 2.32 (3H, s), 2.63 (2H, t), 3.11 (2H, s), 3.21 (4H, m), 3.56 (2H, t), 3.73-3.79 (6H, m), 4.13 (2H, t), 6.85 (2H, d), 6.92 (2H, d), 7.11 (1H, d), 7.96 (1H, d); m/z=505 [M+H]+.

The 1-[2-(4-{4-[3-(trifluoromethyl)[1,2,4]triazolo[4,3-b]pyridazin-6-yl]piperazin-1-yl}phenoxy)ethyl]piperazin-2-one used as starting material was prepared as follows:—

### Preparation of tert-butyl 3-oxo-4-[2-(4-{4-[3-(trifluoromethyl)[1,2,4]triazolo[4,3-b]pyridazin-6-yl]piperazin-1-yl}phenoxy)ethyl]piperazine-1-carboxylate

A solution of tert-butyl 3-oxopiperazine-1-carboxylate (123 mg, 0.62 mmol) in THF (2 mL) was added to a stirred suspension of sodium hydride (60% dispersion in mineral oil) (24.67 mg, 0.62 mmol) in THF (1 mL) under nitrogen. The resulting mixture was stirred at ambient temperature for 15 minutes then a solution of 2-(4-{4-[3-(trifluoromethyl)[1,2,4]triazolo[4,3-b]pyridazin-6-yl]piperazin-1-yl}phenoxy)ethyl methanesulfonate (obtained as described in Example 4, preparation of starting materials) (200 mg, 0.41 mmol) in THF (2 mL) was added and the solution was heated at 65° C. for 18 hours. The reaction mixture was cooled to room temperature, quenched with MeOH (0.5 mL) and the solvents evaporated to give crude product. The crude product was purified by flash silica chromatography, elution gradient 0 to 2% MeOH in DCM. Fractions containing the desired product were evaporated to dryness to give tert-butyl 3-oxo-4-[2-(4-{4-[3-(trifluoromethyl)-[1,2,4]triazolo[4,3-b]pyridazin-6-yl]piperazin-1-yl}phenoxy)ethyl]piperazine-1-carboxylate (86 mg, 35%) as a solid.

m/z=591 [M+H]+.

### Preparation of 1-[2-(4-{4-[3-(trifluoromethyl)[1,2,4]triazolo[4,3-b]pyridazin-6-yl]piperazin-1-yl}phenoxy)ethyl]piperazin-2-one

TFA (1 mL) was added to tert-butyl 3-oxo-4-[2-(4-{4-[3-(trifluoromethyl)-[1,2,4]triazolo[4,3-b]pyridazin-6-yl]piperazin-1-yl}phenoxy)ethyl]piperazine-1-carboxylate (86 mg, 0.15 mmol) in DCM (1 mL). The resulting solution was stirred at ambient temperature for 30 minutes then added to an SCX column. The crude product was eluted from the column using 2M ammonia/MeOH and the solvents were evaporated. The crude product was purified by flash silica chromatography, elution gradient 0 to 5% 2M ammonia/MeOH in DCM. Pure fractions were evaporated to dryness to give 1-[2-(4-{4-[3-(trifluoromethyl)[1,2,4]triazolo[4,3-b]pyridazin-6-yl]piperazin-1-yl}phenoxy)ethyl]piperazin-2-one (50 mg, 70%) as a solid.

m/z=491 [M+H]+.

### Example 8

### Preparation of 4-methyl-1-[2-(4-{1-[3-(trifluoromethyl)[1,2,4]triazolo[4,3-b]pyridazin-6-yl]piperidin-4-yl}phenoxy)ethyl]piperazin-2-one

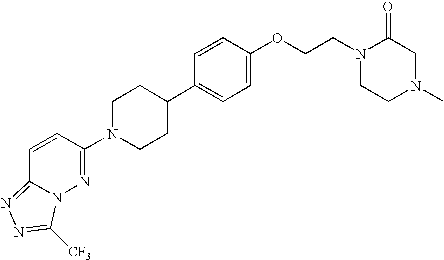

Formaldehyde (37% aqueous solution) (4 mL, 1.09 mmol) was added to 1-[2-(4-{1-[3-(trifluoromethyl)[1,2,4]triazolo[4,3-b]pyridazin-6-yl]piperidin-4-yl}phenoxy)ethyl]piperazin-2-one (534 mg, 1.09 mmol) and acetic acid (0.062 mL, 1.09 mmol) in a mixture of THF (10 mL), DCM (10 mL) and methanol (2 mL). The resulting mixture was stirred at ambient temperature for 30 minutes then sodium triacetoxyhydroborate (694 mg, 3.27 mmol) was added and stirring was continued for a further 30 minutes. The reaction mixture was added to an SCX column and the crude product was eluted from the column using 2M ammonia/MeOH and the solvents were evaporated. The crude product was purified by flash silica chromatography, elution gradient 0 to 5% MeOH in DCM. Pure fractions were evaporated to dryness to give a gum which was triturated with ether. The resulting solid was collected by filtration and dried to give 4-methyl-1-[2-(4-{1-[3-(trifluoromethyl)[1,2,4]triazolo[4,3-b]pyridazin-6-yl]piperidin-4-yl}phenoxy)ethyl]piperazin-2-one (510 mg, 93%) as a solid.

1H NMR (400.1 MHz, DMSO-d6) δ 1.66 (2H, m), 1.88 (2H, m), 2.20 (3H, s), 2.55 (2H, t), 2.81 (1H, m), 2.92 (2H, s), 3.10 (2H, m), 3.42 (2H, t), 3.62 (2H, t), 4.08 (2H, t), 4.41 (2H, m), 6.87 (2H, d), 7.18 (2H, d), 7.65 (1H, d), 8.23 (1H, d); m/z=504 [M+H]+.

The 1-[2-(4-{1-[3-(trifluoromethyl)[1,2,4]triazolo[4,3-b]pyridazin-6-yl]piperidin-4-yl}phenoxy)ethyl]piperazin-2-one used as starting material was prepared as follows:—

### Preparation of tert-butyl 3-oxo-4-(2-(4-(1-(3-(trifluoromethyl)[1,2,4]triazolo[4,3-b]pyridazin-6-yl)piperidin-4-yl)phenoxy)ethyl)piperazine-1-carboxylate

Obtained in 84% yield by an analogous method to Example 7, preparation of starting materials, starting from 2-(4-{1-[3-(trifluoromethyl)[1,2,4]triazolo[4,3-b]pyridazin-6-yl]piperidin-4-yl}phenoxy)ethyl methanesulfonate (obtained as described in Example 2, preparation of starting materials).

m/z=590 [M+H]+.

### Preparation of 1-[2-(4-{1-[3-(trifluoromethyl)[1,2,4]triazolo[4,3-b]pyridazin-6-yl]piperidin-4-yl}phenoxy)ethyl]piperazin-2-one

Obtained in 56% yield by an analogous method to Example 7, preparation of starting materials, starting from tert-butyl 3-oxo-4-(2-(4-(1-(3-(trifluoromethyl)-[1,2,4]triazolo[4,3-b]pyridazin-6-yl)piperidin-4-yl)phenoxy)ethyl)piperazine-1-carboxylate.

1H NMR (399.9 MHz, CDCl3) δ 1.76 (2H, m), 1.99 (2H, m), 2.79 (1H, m), 3.05 (2H, t), 3.11 (2H, m), 3.49-3.55 (4H, m), 3.75 (2H, t), 4.18 (2H, t), 4.37 (2H, m), 6.85 (2H, m), 7.13 (3H, m), 7.92 (1H, d); m/z=490 [M+H]+.

### Examples 9-10

The following compounds were prepared in 43-66% yield by an analogous method to Example 6, starting from 3-(4-{1-[3-(trifluoromethyl)[1,2,4]triazolo[4,3-b]pyridazin-6-yl]piperidin-4-yl}phenoxy)propyl methanesulfonate and the appropriate piperazinone:—

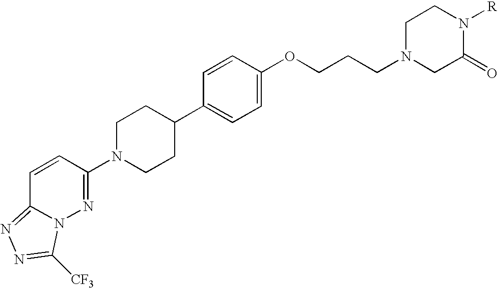

| Ex. | R | 1H NMR (399.9 MHz, CDCl3) | m/z [M + H]+ |
|---|---|---|---|
| 9 | Methyl | δ 1.76 (2H, m), 1.98 (4H, m), 2.60 (2H, m), 2.69- 2.81 (3H, m), 2.95 (3H, s), 3.08-3.21 (4H, m), 3.33 (2H, m), 4.01 (2H, t), 4.37 (2H, m), 6.85 (2H, d), 7.11-7.14 (3H, m), 7.92 (1H, d) | 518 |
| 10 | Cyclopropyl | δ 0.60 (2H, m), 0.74 (2H, m), 1.69 (2H, m), 1.85- 1.95 (4H, m), 2.49 (2H, t), 2.57-2.75 (4H, m), 3.01-3.07 (4H, m), 3.19 (2H, t), 3.93 (2H, t), 4.30 (2H, m), 6.78 (2H, d), 7.05 (3H, m), 7.85 (1H, d) | 544 |

The 3-(4-{1-[3-(trifluoromethyl)[1,2,4]triazolo[4,3-b]pyridazin-6-yl]piperidin-4-yl}phenoxy)propyl methanesulfonate used as starting material was prepared as follows:—

### Preparation of 3-(4-{1-[3-(trifluoromethyl)[1,2,4]triazolo[4,3-b]pyridazin-6-yl]piperidin-4-yl}phenoxy)propan-1-ol

Obtained in 75% yield by an analogous method to Example 1.2, preparation of starting materials, starting from 4-{1-[3-(trifluoromethyl)[1,2,4]triazolo[4,3-b]pyridazin-6-yl]piperidin-4-yl}phenol (obtained as described in Example 2, preparation of starting materials).

1H NMR (399.9 MHz, DMSO-d6) δ 1.64-1.72 (2H, m), 1.81-1.90 (4H, m), 2.77-2.84 (1H, m), 3.07-3.13 (2H, m), 3.53-3.57 (2H, m), 4.00 (2H, t), 4.41 (2H, d), 4.50 (1H, t), 6.84-6.88 (2H, m), 7.17 (2H, d), 7.66 (1H, d), 8.24 (1H, d); m/z=422 [M+H]+.

### Preparation of 3-(4-{1-[3-(trifluoromethyl)[1,2,4]triazolo[4,3-b]pyridazin-6-yl]piperidin-4-yl}phenoxy)propyl methanesulfonate

Obtained in 97% yield by an analogous method to Example 1.2, preparation of starting materials, starting from 3-(4-{1-[3-(trifluoromethyl)[1,2,4]triazolo[4,3-b]pyridazin-6-yl]piperidin-4-yl}phenoxy)propan-1-ol.

1H NMR (399.9 MHz, CDCl3) δ 1.71-1.82 (2H, m), 2.00 (2H, d), 2.19-2.25 (2H, m), 2.76-2.82 (1H, m), 2.99 (3H, s), 3.08-3.15 (2H, m), 4.08 (2H, t), 4.35-4.39 (2H, m), 4.44 (2H, t), 6.84-6.88 (2H, m), 7.12-7.16 (3H, m), 7.93 (1H, d); m/z=500 [M+H]+.

### Example 11

### Preparation of 1-methyl-4-{2-[(5-{1-[3-(trifluoromethyl)[1,2,4]triazolo[4,3-b]pyridazin-6-yl]piperidin-4-yl}pyridin-2-yl)oxy]ethyl}piperazin-2-one

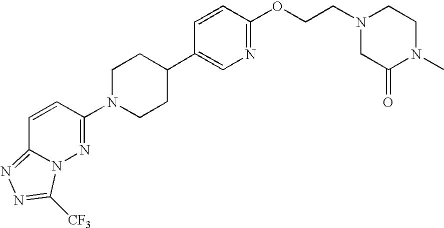

Ammonium formate (257 mg, 4.08 mmol) was added to 1-methyl-4-[2-({1′-[3-(trifluoromethyl)[1,2,4]triazolo[4,3-b]pyridazin-6-yl]-1′,2′,3′,6′-tetrahydro-3,4′-bipyridin-6-yl}oxy)ethyl]piperazin-2-one (205 mg, 0.41 mmol) and 10% palladium on carbon (43.4 mg, 0.04 mmol) in ethanol (10 mL). The resulting mixture was stirred at 78° C. for 30 minutes, cooled to room temperature, and the catalyst removed by filtration and washed with MeOH. The filtrate was evaporated to give crude product, DCM was added to the residue and any insoluble material was removed by filtration, and the filtrate was purified by flash silica chromatography, elution gradient 0 to 5% MeOH in DCM. Pure fractions were evaporated to dryness to afford a gum, which was triturated with ether. The resulting solid was collected by filtration and dried to give 1-methyl-4-{2-[(5-{1-[3-(trifluoromethyl)[1,2,4]triazolo[4,3-b]pyridazin-6-yl]piperidin-4-yl}pyridin-2-yl)oxy]ethyl}piperazin-2-one (80 mg, 39%).

1H NMR (399.9 MHz, CDCl3) δ 1.77 (2H, m), 2.00 (2H, m), 2.77-2.87 (5H, m), 2.94 (3H, s), 3.13 (2H, m), 3.28 (2H, s), 3.33 (2H, t), 4.38 (2H, m), 4.44 (2H, t), 6.74 (1H, d), 7.12 (1H, d), 7.43 (1H, m), 7.94 (1H, d), 8.00 (1H, d); m/z=505 [M+H]+. The 1-methyl-4-[2-({1′-[3-(trifluoromethyl)[1,2,4]triazolo[4,3-b]pyridazin-6-yl]-1′,2′,3′,6′-tetrahydro-3,4′-bipyridin-6-yl}oxy)ethyl]piperazin-2-one used as starting material was prepared as follows:—

### Preparation of 1-[3-(trifluoromethyl)[1,2,4]triazolo[4,3-b]pyridazin-6-yl]piperidin-4-one

6-Chloro-3-(trifluoromethyl)-[1,2,4]triazolo[4,3-b]pyridazine (10.0 g, 44.93 mmol), 4-piperidone monohydrate hydrochloride (8.48 g, 49.43 mmol) and DIPEA (16.3 ml, 98.85 mmol) in DMF (100 ml) were stirred and heated at 90° C. for 1 hour. The DMF was then evaporated in vacuo and the residue purified by flash silica chromatography, eluting with 2% MeOH in DCM. Pure fractions were evaporated to dryness to give a pale yellow precipitate. The precipitate was collected by filtration, washed with diethyl ether and air dried to afford 1-[3-(trifluoromethyl)-[1,2,4]triazolo[4,3-b]pyridazin-6-yl]piperidin-4-one (10.61 g, 83%) as a pale yellow solid.

1H NMR (399.9 MHz, CDCl3) δ 2.65 (4H, t), 3.98 (4H, t), 7.15 (1H, d), 8.02 (1H, d); m/z=286 [M+H]+.

### Preparation of 1-[3-(trifluoromethyl)[1,2,4]triazolo[4,3-b]pyridazin-6-yl]-1,2,3,6-tetrahydropyridin-4-yl trifluoromethanesulfonate

A solution of 1-[3-(trifluoromethyl)-[1,2,4]triazolo[4,3-b]pyridazin-6-yl]piperidin-4-one (5.16 g, 18.09 mmol) in THF (100 mL) was added dropwise to lithium bis(trimethylsilyl)amide (1M in THF) (19.90 mL, 19.90 mmol) at −78° C., over a period of 30 minutes under nitrogen. The resulting yellow suspension was stirred at −78° C. for 20 minutes, then a solution of 1,1,1-trifluoro-N-phenyl-N-(trifluoromethylsulfonyl)methanesulfonamide (7.11 g, 19.90 mmol) in THF (75 ml) was added dropwise over a period of 10 minutes. The mixture was stirred at −78° C. for 30 minutes then allowed to slowly warm to room temperature and stirred over the weekend. The reaction mixture was quenched with saturated NH₄Cl (5 mL), then concentrated and diluted with DCM (250 mL), washed with water (150 mL) then brine (150 mL), and evaporated to afford crude product. DCM (50 mL) was added to the crude product, the precipitate was collected by filtration and the filtrate was purified by flash silica chromatography, elution gradient 50 to 80% EtOAc in isohexane. Pure fractions were evaporated to dryness and combined with the previously collected precipitate to give 1-[3-(trifluoromethyl)-[1,2,4]triazolo[4,3-b]pyridazin-6-yl]-1,2,3,6-tetrahydropyridin-4-yl trifluoromethanesulfonate (5.90 g, 78%) as a solid.

1H NMR (399.9 MHz, DMSO-d6) δ 2.43 (2H, m), 3.70 (2H, m), 4.05 (2H, m), 5.99 (1H, m), 7.47 (1H, d), 8.13 (1H, d); m/z=418 [M+H]+.

### Preparation of 5-{1-[3-(trifluoromethyl)[1,2,4]triazolo[4,3-b]pyridazin-6-yl]-1,2,3,6-tetrahydropyridin-4-yl}pyridin-2-ol

Sodium carbonate (2.134 g, 20.13 mmol) was added to 1-[3-(trifluoromethyl)-[1,2,4]triazolo[4,3-b]pyridazin-6-yl]-1,2,3,6-tetrahydropyridin-4-yl trifluoromethanesulfonate (2.8 g, 6.71 mmol) and 5-(4,4,5,5-tetramethyl-1,3,2-dioxaborolan-2-yl)pyridin-2-ol (1.557 g, 7.05 mmol) in a mixture of DME (40 mL) and water (10 mL). The suspension was bubbled with nitrogen for 5 minutes then 1,1′-bis(diphenylphosphino)ferrocenedichloropalladium(II) (0.486 g, 0.67 mmol) was added and the resulting suspension was stirred at 80° C. for 30 minutes. The reaction mixture was cooled to room temperature. The precipitate which had formed in the reaction was collected by filtration then stirred in water (50 mL) for 20 minutes. The solid was collected by filtration, washed with water then ether and dried under vacuum to give 5-{1-[3-(trifluoromethyl)-[1,2,4]triazolo[4,3-b]pyridazin-6-yl]-1,2,3,6-tetrahydropyridin-4-yl}pyridin-2-ol (1.570 g, 64.6%) as a solid.

1H NMR (399.9 MHz, DMSO-d6) δ 3.84 (2H, m), 4.21 (2H, m), 6.15 (1H, s), 6.37 (1H, d), 7.36 (1H, s), 7.66 (1H, d), 7.75 (1H, m), 8.28 (1H, d), 11.60 (1H, s) (2H obscured by DMSO); m/z=363 [M+H]+.

### Preparation of 1-methyl-4-[2-({1′43-(trifluoromethyl)[1,2,4]triazolo[4,3-b]pyridazin-6-yl]-1′,2′,3′,6′-tetrahydro-3,4′-bipyridin-6-yl}oxy)ethyl]piperazin-2-one

DIAD (0.245 mL, 1.24 mmol) was added dropwise to 5-{1-[3-(trifluoromethyl)-[1,2,4]triazolo[4,3-b]pyridazin-6-yl]-1,2,3,6-tetrahydropyridin-4-yl}pyridin-2-ol (300 mg, 0.83 mmol), 4-(2-hydroxyethyl)-1-methylpiperazin-2-one (obtained as described in Example 3, preparation of starting materials) (196 mg, 1.24 mmol) and triphenylphosphine (326 mg, 1.24 mmol) in THF (3 mL) under nitrogen. The resulting suspension was stirred at ambient temperature for 16 hours then the solvent was evaporated. The crude product was purified by flash silica chromatography, elution gradient 80 to 100% EtOAc in isohexane then 0 to 5% MeOH in DCM. Pure fractions were evaporated to dryness to give 1-methyl-4-[2-({1′-[3-(trifluoromethyl)[1,2,4]triazolo[4,3-b]pyridazin-6-yl]-1′,2′,3′,6′-tetrahydro-3,4′-bipyridin-6-yl}oxy)ethyl]piperazin-2-one (205 mg, 49.3%) as a solid.

m/z=503 [M+H]+.

### Example 12

### Preparation of 4-[2-(2-fluoro-4-{4-hydroxy-1-[3-(trifluoromethyl)[1,2,4]triazolo[4,3-b]pyridazin-6-yl]piperidin-4-yl}phenoxy)ethyl]-1-methylpiperazin-2-one

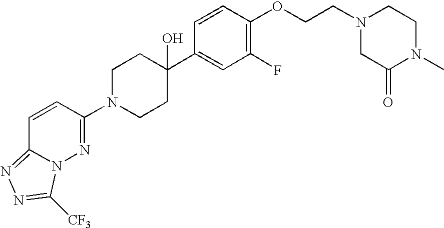

Obtained in 61% yield by an analogous method to Example 3, starting from 4-(3-fluoro-4-hydroxyphenyl)-1-[3-(trifluoromethyl)[1,2,4]triazolo[4,3-b]pyridazin-6-yl]piperidin-4-ol and 4-(2-hydroxyethyl)-1-methylpiperazin-2-one.

1H NMR (399.9 MHz, CDCl3) δ 1.91 (2H, m), 2.10 (2H, m), 2.88 (4H, m), 2.95 (3H, s), 3.27 (2H, s), 3.34 (2H, t), 3.57 (2H, m), 4.13-4.19 (4H, m), 6.95 (1H, m), 7.12-7.16 (2H, m), 7.23 (1H, m), 7.93 (1H, d); m/z=538 [M+H]+.

The 4-(3-fluoro-4-hydroxyphenyl)-1-[3-(trifluoromethyl)[1,2,4]triazolo[4,3-b]pyridazin-6-yl]piperidin-4-ol used as starting material was prepared as follows:—

### Preparation of 1-(benzyloxy)-4-bromo-2-fluorobenzene

Benzyl bromide (17.12 mL, 143.98 mmol) was added dropwise to 4-bromo-2-fluorophenol (25 g, 130.89 mmol) and potassium carbonate (36.2 g, 261.78 mmol) in DMF (250 mL). The resulting mixture was stirred at ambient temperature for 3 days. The reaction mixture was diluted with ether (300 mL), and the solution was washed sequentially with water (2×500 mL) and saturated brine (250 mL). The organic layer was dried over MgSO4, filtered and evaporated to afford an oil which was scratched to give a solid. The solid was triturated with isohexane, filtered and dried to give 1-(benzyloxy)-4-bromo-2-fluorobenzene (34 g, 93%) as a solid.

1H NMR (399.9 MHz, DMSO-d6) δ 5.19 (2H, s), 7.24 (1H, m), 7.32-7.47 (6H, m), 7.54 (1H, m).

### Preparation of benzyl 4-[4-(benzyloxy)-3-fluorophenyl]-4-hydroxypiperidine-1-carboxylate

Obtained in 57% yield by an analogous method to Example 3, preparation of starting materials, starting from benzyl 4-oxopiperidine-1-carboxylate and 1-(benzyloxy)-4-bromo-2-fluorobenzene.

1H NMR (399.9 MHz, DMSO-d6) δ 1.58 (2H, m), 1.82 (2H, m), 3.21 (2H, m), 3.93 (2H, m), 5.10 (2H, s), 5.14 (1H, s), 5.17 (2H, s), 7.15-7.21 (2H, m), 7.29-7.47 (11H, m); m/z=434 [M−H]+.

### Preparation of 4-(3-fluoro-4-hydroxyphenyl)piperidin-4-ol

Obtained in 66% yield by an analogous method to Example 3, preparation of starting materials, starting from benzyl 4-[4-(benzyloxy)-3-fluorophenyl]-4-hydroxypiperidine-1-carboxylate.

1H NMR (399.9 MHz, DMSO-d6) δ 1.48 (2H, m), 1.72 (2H, m), 2.70 (2H, m), 2.89 (2H, m), 4.67 (1H, s), 6.87 (1H, m), 7.03 (1H, m), 7.16 (1H, m).

### Preparation of 4-(3-fluoro-4-hydroxyphenyl)-1-[3-(trifluoromethyl)[1,2,4]triazolo[4,3-b]pyridazin-6-yl]piperidin-4-ol

Obtained in 96% yield by an analogous method to Example 3, preparation of starting materials, starting from 6-chloro-3-(trifluoromethyl)-[1,2,4]triazolo[4,3-b]pyridazine and benzyl 4-[4-(benzyloxy)-3-fluorophenyl]-4-hydroxypiperidine-1-carboxylate.

1H NMR (399.9 MHz, DMSO-d6) δ 1.70 (2H, m), 1.97 (2H, m), 3.41 (2H, m), 4.18 (2H, m), 5.16 (1H, s), 6.88 (1H, m), 7.09 (1H, m), 7.24 (1H, m), 7.65 (1H, d), 8.23 (1H, d), 9.63 (1H, s); m/z=398 [M+H]+.

### Example 13

### Preparation of 4-[2-(2-fluoro-4-{1-[3-(trifluoromethyl)[1,2,4]triazolo[4,3-b]pyridazin-6-yl]piperidin-4-yl}phenoxy)ethyl]-1-methylpiperazin-2-one

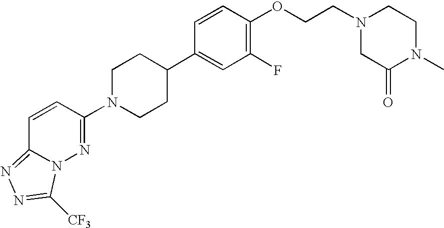

Obtained in 74% yield by an analogous method to Example 3, starting from 2-fluoro-4-{1-[3-(trifluoromethyl)[1,2,4]triazolo[4,3-b]pyridazin-6-yl]piperidin-4-yl}phenol and 4-(2-hydroxyethyl)-1-methylpiperazin-2-one.

1H NMR (399.9 MHz, CDCl3) δ 1.67 (2H, m), 1.93 (2H, m), 2.71 (1H, m), 2.81 (4H, m), 2.88 (3H, s), 3.04 (2H, m), 3.21 (2H, s), 3.27 (2H, t), 4.09 (2H, t), 4.30 (2H, m), 6.82-6.89 (3H, m), 7.05 (1H, d), 7.86 (1H, d); m/z=522 [M+H]+.

The 2-fluoro-4-{1-[3-(trifluoromethyl)[1,2,4]triazolo[4,3-b]pyridazin-6-yl]piperidin-4-yl}phenol used as starting material was prepared as follows:—

### Preparation of 6-{4-[4-(benzyloxy)-3-fluorophenyl]-5,6-dihydropyridin-1(2H)-yl}-3-(trifluoromethyl)[1,2,4]triazolo[4,3-b]pyridazine

Sodium carbonate (2.179 g, 20.56 mmol) was added to 1-[3-(trifluoromethyl)-[1,2,4]triazolo[4,3-b]pyridazin-6-yl]-1,2,3,6-tetrahydropyridin-4-yl trifluoromethanesulfonate (obtained as described in Example 11, preparation of starting materials) (2.86 g, 6.85 mmol) and 4-(benzyloxy)-3-fluorophenylboronic acid (1.771 g, 7.20 mmol) in a mixture of DME (40 mL) and water (10 mL). The suspension was bubbled with nitrogen for 5 minutes then 1,1′-bis(diphenylphosphino)ferrocenedichloropalladium(II) (0.496 g, 0.69 mmol) was added and the resulting suspension was stirred at 80° C. for 1 hour. The reaction mixture was cooled to room temperature, diluted with DCM (250 mL) and washed with water (2×250 mL). The organic layer was dried over MgSO4, filtered and evaporated to afford crude product. The crude product was purified by flash silica chromatography, elution gradient 50 to 70% EtOAc in isohexane. Pure fractions were evaporated to dryness to give 6-{4-[4-(benzyloxy)-3-fluorophenyl]-5,6-dihydropyridin-1(2H)-yl}-3-(trifluoromethyl)-[1,2,4]triazolo[4,3-b]pyridazine (2.39 g, 74%) as a solid.

1H NMR (399.9 MHz, DMSO-d6) δ 2.63 (2H, m), 3.87 (2H, m), 4.24 (2H, m), 5.21 (2H, s), 6.29 (1H, m), 7.24 (2H, m), 7.33-7.48 (6H, m), 7.67 (1H, d), 8.28 (1H, d); m/z=470 [M+H]+.

### Preparation of 2-fluoro-4-{1-[3-(trifluoromethyl)[1,2,4]triazolo[4,3-b]pyridazin-6-yl]piperidin-4-yl}phenol

Ammonium formate (3.21 g, 50.91 mmol) was added to 6-(4-(4-(benzyloxy)-3-fluorophenyl)-5,6-dihydropyridin-1(2H)-yl)-3-(trifluoromethyl)[1,2,4]triazolo[4,3-b]pyridazine (2.39 g, 5.09 mmol) and palladium (10 wt % on carbon) (0.542 g, 0.51 mmol) in ethanol (50 mL). The resulting mixture was stirred at 78° C. for 1 hour then cooled to room temperature. The catalyst was removed by filtration, washed with MeOH and the solvents evaporated. The residue was dissolved in 25% MeOH in DCM (200 mL) and washed with water (100 mL) followed by saturated brine (100 mL). The organic layer was dried over MgSO4, filtered and evaporated to give 2-fluoro-4-{1-[3-(trifluoromethyl)[1,2,4]triazolo[4,3-b]pyridazin-6-yl]piperidin-4-yl}phenol (1.54 g, 67%) as a solid.

1H NMR (399.9 MHz, DMSO-d6) δ 1.64 (2H, m), 1.88 (2H, m), 2.78 (1H, m), 3.08 (2H, m), 4.40 (2H, m), 6.87 (2H, m), 7.04 (1H, m), 7.65 (1H, d), 8.24 (1H, d), 9.57 (1H, s); m/z=380 [M−H]+.

### Example 14

### Preparation of 6-(4-{4-[2-(4-acetyl-1,4-diazepan-1-yl)ethoxy]phenyl}piperidin-1-yl)-3-(trifluoromethyl)[1,2,4]-triazolo[4,3-b]pyridazine

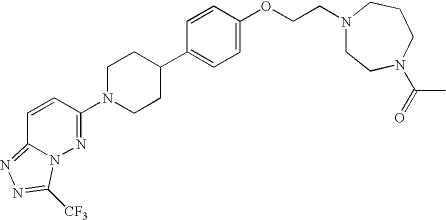

Obtained in 64% yield by an analogous method to Example 6, starting from 2-(4-{1-[3-(trifluoromethyl)[1,2,4]triazolo[4,3-b]pyridazin-6-yl]piperidin-4-yl}phenoxy)ethyl methanesulfonate and N-acetylhomopiperazine.

1H NMR (499.803 MHz, DMSO-d6 at 373K) δ 1.65-1.80 (4H, m), 1.93 (2H, m), 1.97 (3H, s), 2.70-2.88 (5H, m), 3.14 (2H, m), 3.48 (4H, m), 4.04 (2H, t), 4.37 (2H, m), 6.86 (2H, d), 7.16 (2H, d), 7.54 (1H, d), 8.13 (1H, d) (2H obscured by DMSO and water); m/z=532 [M+H]+.

### Example 15

### Preparation of 1-ethyl-4-[3-(4-{1-[3-(trifluoromethyl)[1,2,4]triazolo[4,3-b]pyridazin-6-yl]piperidin-4-yl}phenoxy)propyl]piperazin-2-one

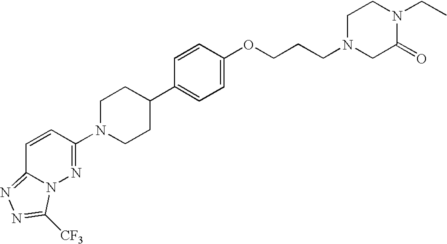

A solution of 3-(4-{1-[3-(trifluoromethyl)[1,2,4]triazolo[4,3-b]pyridazin-6-yl]piperidin-4-yl}phenoxy)propyl methanesulfonate (obtained as described in Example 9, preparation of starting materials) (0.27 g, 0.54 mmol) and 1-ethylpiperazin-2-one (0.159 g, 1.24 mmol) in DMF (20 mL) was stirred at 65° C. for 1 hour. The solvent was evaporated and the residue was quenched with water (50 mL) and extracted with EtOAc (3×50 mL). The organic layer was dried over MgSO4, filtered and evaporated to afford a yellow solid. The crude product was purified by flash silica chromatography, elution gradient 0 to 3% MeOH in DCM. Pure fractions were evaporated to dryness to afford a yellow gum, which was dissolved in a small amount of DCM. Diethyl ether was added and the mixture was stirred for 10 minutes to give a solid, which was collected by filtration and dried under vacuum to give 1-ethyl-4-[3-(4-{1-[3-(trifluoromethyl)[1,2,4]triazolo[4,3-b]pyridazin-6-yl]piperidin-4-yl}phenoxy)propyl]piperazin-2-one (0.138 g, 48.0%) as a white solid.

1H NMR (400.1 MHz, CDCl3) δ 1.14 (3H, t), 1.82-1.71 (2H, m), 2.01-1.92 (4H, m), 2.57 (2H, t), 2.70 (2H, t), 2.80-2.76 (1H, m), 3.14-3.08 (4H, m), 3.31 (2H, t), 3.43 (2H, q), 4.01 (2H, t), 4.38-4.35 (2H, m), 6.86 (2H, d), 7.13-7.11 (3H, m), 7.92 (1H, d); m/z=532 [M+H]+.

### Examples 16-17

The following compounds were prepared in 48-75% yield by an analogous method to Example 15, starting from 3-(4-{1-[3-(trifluoromethyl)[1,2,4]triazolo[4,3-b]pyridazin-6-yl]piperidin-4-yl}phenoxy)propyl methanesulfonate and the appropriate piperazinone:—

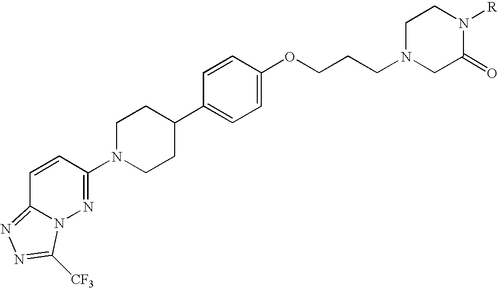

| Ex. | R | 1H NMR (400.1 MHz, CDCl3) | m/z [M + H]+ |
|---|---|---|---|
| 16 | Isopropyl | δ 1.12 (6H, d), 1.76 (2H, ddd), 2.01-1.92 (4H, m), 2.57 (2H, t), 2.67 (2H, t), 2.81- 2.75 (1H, m), 3.16-3.08 (4H, m), 3.22 (2H, t), 4.01 (2H, t), 4.38-4.35 (2H, m), 4.86 (1H, septet), 6.85 (2H, d), 7.13-7.11 (3H, m), 7.92 (1H, d) | 546 |
| 17 | 2-Methoxyethyl | δ 1.81-1.71 (2H, m), 2.01-1.94 (4H, m), 2.58 (2H, t), 2.68 (2H, t), 2.81-2.75 (1H, m), 3.16-3.08 (4H, m), 3.33 (3H, s), 3.46- 3.43 (2H, m), 3.56 (4H, s), 4.01 (2H, t), 4.38-4.35 (2H, m), 6.86-6.84 (2H, m), 7.13-7.11 (3H, m), 7.92 (1H, d) | 562 |

### Example 18

### Preparation of 1-ethyl-4-[2-(4-{1-[3-(trifluoromethyl)[1,2,4]triazolo[4,3-b]pyridazin-6-yl]piperidin-4-yl}phenoxy)ethyl]piperazin-2-one

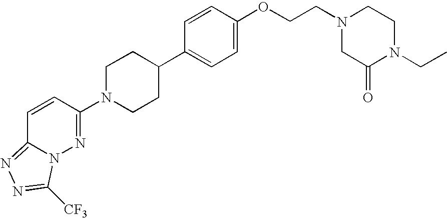

A solution of 6-{4-[4-(2-bromoethoxy)phenyl]piperidin-1-yl}-3-(trifluoromethyl)[1,2,4]triazolo[4,3-b]pyridazine (0.20 g, 0.43 mmol) and 1-ethylpiperazin-2-one (0.159 g, 1.24 mmol) in DMF (20 mL) was stirred at 50° C. overnight. The solvent was evaporated and the residue was purified by flash silica chromatography, elution gradient 0 to 5% MeOH in DCM. Pure fractions were evaporated to dryness to afford a clear gum which was dissolved in a small amount of DCM. Diethyl ether was added and the system was sonicated to afford a solid, which was collected by filtration and dried under vacuum to give 1-ethyl-4-[2-(4-{1-[3-(trifluoromethyl)[1,2,4]triazolo[4,3-b]pyridazin-6-yl]piperidin-4-yl}phenoxy)ethyl]piperazin-2-one (156 mg, 70%) as a white solid.

1H NMR (400.1 MHz, CDCl3) δ 1.14 (3H, t), 1.76 (2H, ddd), 2.01-1.98 (2H, m), 2.86-2.73 (5H, m), 3.14-3.08 (2H, m), 3.27 (2H, s), 3.33 (2H, t), 3.43 (2H, q), 4.10 (2H, t), 4.38-4.35 (2H, m), 6.88-6.85 (2H, m), 7.14-7.11 (3H, m), 7.92 (1H, d); m/z=518 [M+H]+.

The 6-{4-[4-(2-bromoethoxy)phenyl]piperidin-1-yl}-3-(trifluoromethyl)[1,2,4]triazolo[4,3-b]pyridazine used as starting material was prepared as follows:—

A mixture of 2-(4-{1-[3-(trifluoromethyl)[1,2,4]triazolo[4,3-b]pyridazin-6-yl]piperidin-4-yl}phenoxy)ethyl methanesulfonate (obtained as described in Example 2, preparation of starting materials) (0.96 g, 1.98 mmol) and lithium bromide (0.859 g, 9.89 mmol) in dioxan (5 mL) was heated at 100° C. for 4 hours. The reaction mixture was quenched with water (50 mL) and stirred for 30 minutes to afford a solid, which was collected by filtration and dried under vacuum to give 6-{4-[4-(2-bromoethoxy)phenyl]piperidin-1-yl}-3-(trifluoromethyl)[1,2,4]triazolo[4,3-b]pyridazine (0.880 g, 95%) as a white solid.

1H NMR (400.1 MHz, CDCl3) δ 1.76 (2H, ddd), 2.02-1.98 (2H, m), 2.82-2.75 (1H, m), 3.12 (2H, ddd), 3.62 (2H, t), 4.28 (2H, t), 4.38-4.35 (2H, m), 6.89-6.87 (2H, m), 7.16-7.11 (3H, m), 7.92 (1H, d); m/z=471 [M+H]+.

### Examples 19-20

The following compounds were prepared in 70-73% yield by an analogous method to Example 18, starting from 6-{4-[4-(2-bromoethoxy)phenyl]piperidin-1-yl}-3-(trifluoromethyl)[1,2,4]triazolo[4,3-b]pyridazine and the appropriate piperazinone:—

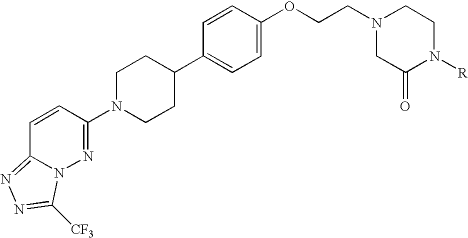

| Ex. | R | 1H NMR (400.1 MHz, CDCl3) | m/z [M + H]+ |
|---|---|---|---|
| 19 | Isopropyl | δ 1.12 (6H, d), 1.76 (2H, ddd), 2.01-1.98 (2H, m), 2.86-2.75 (5H, m), 3.15-3.08 (2H, m), 3.25-3.23 (2H, m), 3.29 (2H, s), 4.10 (2H, t), 4.38-4.35 (2H, m), 4.86 (1H, quintet), 6.88- 6.85 (2H, m), 7.14-7.11 (3H, m), 7.92 (1H, d) | 532 |
| 20 | 2-Methoxyethyl | δ 1.76 (2H, ddd), 2.01-1.98 (2H, m), 2.87-2.75 (5H, m), 3.15-3.08 (2H, m), 3.29 (2H, s), 3.33 (3H, s), 3.47 (2H, t), 3.55 (4H, s), 4.10 (2H, t), 4.38-4.35 (2H, m), 6.88-6.85 (2H, m), 7.14- 7.11 (3H, m), 7.92 (1H, d) | 548 |

### Example 21

### Preparation of 2-oxo-2-{4-[2-(4-{1-[3-(trifluoromethyl)[1,2,4]triazolo[4,3-b]pyridazin-6-yl]piperidin-4-yl}phenoxy)ethyl]piperazin-1-yl]ethanol

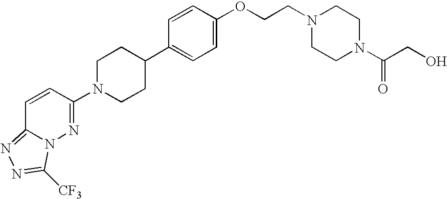

DIPEA (0.299 mL, 1.71 mmol) was added to a stirred solution of 6-{4-[4-(2-piperazin-1-ylethoxy)phenyl]piperidin-1-yl}-3-(trifluoromethyl)[1,2,4]triazolo[4,3-b]pyridazine trihydrochloride (200 mg, 0.34 mmol), 2-hydroxyacetic acid (28.6 mg, 0.38 mmol) and HATU (143 mg, 0.38 mmol) in DMF (2.5 mL). The mixture was stirred for 15 minutes. The crude mixture was purified by preparative HPLC (Waters XBridge Prep C18 OBD column, 5μ silica, 19 mm diameter, 100 mm length), using decreasingly polar mixtures of water (containing 1% ammonia) and MeCN as eluents. Fractions containing the desired compound were evaporated to dryness to afford 2-oxo-2-{4-[2-(4-{1-[3-(trifluoromethyl)[1,2,4]triazolo[4,3-b]pyridazin-6-yl]piperidin-4-yl}phenoxy)ethyl]piperazin-1-yl}ethanol (93 mg, 51.0%) as a white foam. 1H NMR (399.9 MHz, DMSO-d₆) δ 1.64-1.72 (2H, m), 1.88 (2H, d), 2.46-2.48 (4H, m), 2.72 (2H, t), 2.78-2.84 (1H, m), 3.07-3.13 (2H, m), 3.34 (2H, s), 3.47 (2H, s), 4.05-4.08 (4H, m), 4.41 (2H, d), 4.50 (1H, t), 6.86-6.90 (2H, m), 7.16-7.20 (2H, m), 7.66 (1H, d), 8.24 (1H, d); m/z=534 [M+H]+.

The 6-{4-[4-(2-piperazin-1-ylethoxy)phenyl]piperidin-1-yl}-3-(trifluoromethyl)[1,2,4]triazolo[4,3-b]pyridazine trihydrochloride used as starting material was prepared as follows:—

### Preparation of tert-butyl 4-[2-[4-(1-(benzyloxycarbonyl)-1,2,3,6-tetrahydropyridin-4-yl)phenoxy]ethyl]piperazine-1-carboxylate

DIAD (12.60 mL, 64.00 mmol) was added dropwise to benzyl 4-(4-hydroxyphenyl)-5,6-dihydropyridine-1(2H)-carboxylate (obtained as described in Example 2, preparation of starting materials) (16.5 g, 53.34 mmol), tert-butyl 4-(2-hydroxyethyl)piperazine-1-carboxylate (CAS 77279-24-4) (14.74 g, 64.00 mmol) and triphenylphosphine (16.79 g, 64.00 mmol) in THF (150 mL) under nitrogen. The resulting solution was stirred at ambient temperature for 16 hours. The reaction mixture was evaporated to dryness then the residue was stirred in ether (200 mL) for 10 minutes at room temperature. The resulting precipitate was removed by filtration and discarded. The ether filtrate was washed with water (100 mL) followed by saturated brine (100 mL), then dried over MgSO4, filtered and evaporated to give crude product. The crude product was purified by flash silica chromatography, elution gradient 20 to 60% EtOAc in isohexane. Fractions containing the desired product were evaporated to dryness to afford tert-butyl 4-[2-[4-(1-(benzyloxycarbonyl)-1,2,3,6-tetrahydropyridin-4-yl)phenoxy]ethyl]piperazine-1-carboxylate (34.6 g, 82%) as a gum which was contaminated with 34% by weight triphenylphosphine oxide.

1H NMR (399.9 MHz, DMSO-d6) δ 1.40 (9H, s), 2.42-2.47 (6H, m), 2.71 (2H, m), 3.32 (4H, m), 3.62 (2H, m), 4.03-4.10 (4H, m), 5.12 (2H, s), 6.06 (1H, m), 6.92 (2H, d), 7.31-7.40 (7H, m); m/z=522 [M+H]+.

### Preparation of tert-butyl 4-[2-[4-(piperidin-4-yl)phenoxy]ethyl]piperazine-1-carboxylate

tert-Butyl 4-[2-[4-(1-(benzyloxycarbonyl)-1,2,3,6-tetrahydropyridin-4-yl)phenoxy]ethyl]piperazine-1-carboxylate (66% pure by weight) (34.62 g, 43.80 mmol) and 5% palladium on carbon (50% wet) (4.47 g, 1.05 mmol) in MeOH (250 mL) were stirred under an atmosphere of hydrogen at 5 bar and 60° C. for 4 hours. The catalyst was removed by filtration and the solvents evaporated to give crude product. The crude product was purified by flash silica chromatography, eluting with 60% EtOAc in isohexane then 15% 2M ammonia/MeOH in DCM. Pure fractions were evaporated to dryness to afford tert-butyl 4-[2-[4-(piperidin-4-yl)phenoxy]ethyl]piperazine-1-carboxylate (15.42 g, 90%) as a solid.

1H NMR (399.9 MHz, CDCl3) δ 1.46 (9H, s), 1.62 (2H, m), 1.81 (2H, m), 2.50-2.59 (5H, m), 2.73 (2H, m), 2.80 (2H, t), 3.18 (2H, m), 3.44 (4H, m), 4.09 (2H, t), 6.85 (2H, d), 7.13 (2H, d); m/z=390 [M+H]+.

### Preparation of tert-butyl 4-[2-[4-[1-(3-(trifluoromethyl)[1,2,4]triazolo[4,3-b]pyridazin-6-yl]piperidin-4-yl]phenoxy]ethyl]piperazine-1-carboxylate

DIPEA (2.348 mL, 13.48 mmol) was added to 6-chloro-3-(trifluoromethyl)-[1,2,4]triazolo[4,3-b]pyridazine (obtained as described in Monatsh. Chem. 1972, 103, 1591) (2 g, 8.99 mmol) and tert-butyl 4-[2-[4-(piperidin-4-yl)phenoxy]ethyl]piperazine-1-carboxylate (3.68 g, 9.44 mmol) in DMF (30 mL). The resulting solution was stirred at 80° C. for 2 hours. The reaction mixture was cooled to room temperature and the solvents evaporated to dryness. The resulting solid was triturated with water then collected by filtration, washed with ether and dried to afford tert-butyl 4-[2-[4-[1-(3-(trifluoromethyl)-[1,2,4]triazolo[4,3-b]pyridazin-6-yl]piperidin-4-yl]phenoxy]ethyl]piperazine-1-carboxylate (5.02 g, 97%) as a solid.

1H NMR (399.9 MHz, CDCl3) δ 1.46 (9H, s), 1.76 (2H, m), 2.00 (2H, m), 2.54 (4H, m), 2.75-2.86 (3H, m), 3.11 (2H, m), 3.46 (4H, m), 4.11 (2H, m), 4.37 (2H, m), 6.87 (2H, d), 7.13 (3H, m), 7.92 (1H, d); m/z=576 [M+H]+.

### Preparation of 6-{4-[4-(2-piperazin-1-ylethoxy)phenyl]piperidin-1-yl}-3-(trifluoromethyl)[1,2,4]triazolo[4,3-b]pyridazine trihydrochloride

4.0M HCl in dioxane (36.0 ml, 144.02 mmol) was added to a stirred suspension of tert-butyl 4-[2-[4-[1-(3-(trifluoromethyl)-[1,2,4]triazolo[4,3-b]pyridazin-6-yl]piperidin-4-yl]phenoxy]ethyl]piperazine-1-carboxylate (8.29 g, 14.40 mmol) in dioxane (166 ml). The reaction mixture was stirred at room temperature for 48 hours. The product was isolated by filtration, washed with dioxane and diethyl ether and vacuum dried to afford 6-{4-[4-(2-piperazin-1-ylethoxy)phenyl]piperidin-1-yl}-3-(trifluoromethyl)[1,2,4]triazolo[4,3-b]pyridazine trihydrochloride as an off-white solid (8.155 g, 13.94 mmol, 97%).

1H NMR (399.9 MHz, DMSO-d6) δ 1.61-1.74 (2H, m), 1.85-1.92 (2H, m), 2.77-2.88 (1H, m), 3.11 (2H, t), 3.61 (2H, t), 4.40-4.81 (8H, m), 4.41 (4H, t), 6.96 (2H, d), 7.23 (2H, d), 7.67 (1H, q), 8.25 (1H, d), 9.81 (2H, s), 12.18 (1H, s); m/z=476 [M+H]+.

### Examples 22-25

The following compounds were prepared in 28-60% yield by an analogous method to Example 21, starting from 6-{4-[4-(2-piperazin-1-ylethoxy)phenyl]piperidin-1-yl}-3-(trifluoromethyl)[1,2,4]triazolo[4,3-b]pyridazine and the appropriate carboxylic acid:—

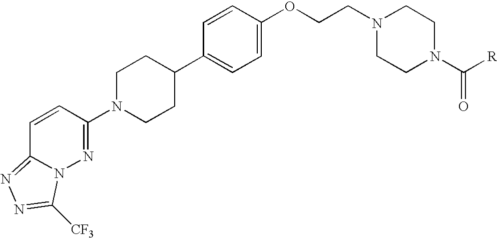

| Ex. | 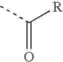 | 1H NMR (399.9 MHz, DMSO-d6) | m/z [M + H]+ |
|---|---|---|---|
| 22 | 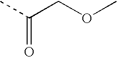 | δ 1.62-1.71 (2H, m), 1.88 (2H, d), 2.43-2.51 (4H, m), 2.71 (2H, t), 2.81 (1H, t), 3.10 (2H, t), 3.30 (3H, s), 3.37-3.40 (2H, m), 3.36-3.44 (2H, m), 4.05-4.08 (4H, m), 4.41 (2H, d), 6.87-6.89 (2H, m), 7.17-7.19 (2H, m), 7.66 (1H, d), 8.23- 8.26 (1H, m) | 548 |
| 23 | 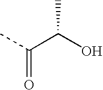 | δ 1.18 (3H, d), 1.61-1.72 (2H, m), 1.88 (2H, d), 2.45-2.48 (4H, m), 2.72 (2H, t), 2.81 (1H, q), 3.07-3.13 (2H, m), 3.40-3.51 (4H, m), 4.07 (2H, t), 4.38-4.45 (3H, m), 4.82 (1H, d), 6.86- 6.90 (2H, m), 7.16-7.19 (2H, m), 7.66 (1H, d), 8.24 (1H, d) | 548 |
| 24 | 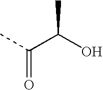 | δ 1.18 (3H, d), 1.61-1.72 (2H, m), 1.88 (2H, d), 2.45-2.48 (4H, m), 2.72 (2H, t), 2.81 (1H, q), 3.07-3.13 (2H, m), 3.40-3.51 (4H, m), 4.07 (2H, t), 4.38-4.45 (3H, m), 4.82 (1H, d), 6.86- 6.90 (2H, m), 7.16-7.19 (2H, m), 7.66 (1H, d), 8.24 (1H, d) | 548 |
| 25 | 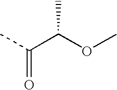 | δ 1.21 (3H, d), 1.61-1.72 (2H, m), 1.88 (2H, d), 2.46-2.53 (4H, m), 2.71 (2H, t), 2.77-2.85 (1H, m), 3.07-3.13 (2H, m), 3.19 (3H, s), 3.47-3.54 (4H, m), 4.07 (2H, t), 4.20 (1H, q), 4.41 (2H, d), 6.86-6.90 (2H, m), 7.18 (2H, s), 7.66 (1H, d), 8.24 (1H, d) | 562 |

### Example 26

### Preparation of 6-(4-{4-[(1R)-2-(4-acetylpiperazin-1-yl)-1-methylethoxy]phenyl}piperidin-1-yl)-3-(trifluoromethyl)[1,2,4]triazolo[4,3-b]pyridazine

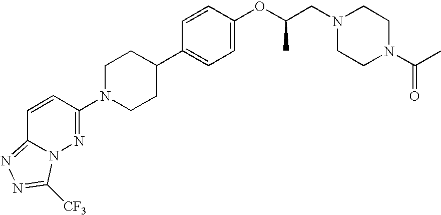

DIAD (0.401 mL, 2.06 mmol) in THF (2 mL) was added dropwise to 4-{1-[3-(trifluoromethyl)[1,2,4]triazolo[4,3-b]pyridazin-6-yl]piperidin-4-yl}phenol (obtained as described in Example 2, preparation of starting materials) (250 mg, 0.69 mmol), (2S)-1-(4-acetylpiperazin-1-yl)propan-2-ol (384 mg, 2.06 mmol) and triphenylphosphine (541 mg, 2.06 mmol) in THF (5 mL) at 0° C. under nitrogen. The resulting solution was stirred at 0° C. for 15 minutes and then at room temperature for 21 hours. The reaction mixture was diluted with ethyl acetate (20 mL) and washed with saturated sodium bicarbonate (20 mL) and saturated brine (20 mL). The organic layer was dried (MgSO4), filtered and evaporated to a gum. The crude product was purified by flash silica chromatography, elution gradient 0 to 4% MeOH in DCM. Pure fractions were evaporated to afford 6-(4-{4-[(1R)-2-(4-acetylpiperazin-1-yl)-1-methylethoxy]phenyl}piperidin-1-yl)-3-(trifluoromethyl)[1,2,4]triazolo[4,3-b]pyridazine (199 mg, 54%) as a dry film.

1H NMR (400.1 MHz, DMSO-d6); δ 1.22 (3H, d), 1.61-1.71 (2H, m), 1.86-1.90 (2H, m), 1.97 (3H, s), 2.38-2.61 (6H, m), 2.77-2.83 (1H, m), 3.07-3.13 (2H, m), 3.37-3.39 (4H, m), 4.39-4.42 (2H, m), 4.55-4.62 (1H, m), 6.87 (2H, d), 7.16 (2H, d), 7.65 (1H, d), 8.24 (1H, d); m/z=533 [M+H]+.

The (2S)-1-(4-acetylpiperazin-1-yl)propan-2-ol used as starting material was prepared as follows:—

(S)-2-Methyloxirane (1.246 g, 21.45 mmol) was added to N-acetylpiperazine (2.5 g, 19.50 mmol) in MeOH (50 mL). The resulting solution was stirred at 80° C. for 4 hours, then the solvent was evaporated to afford crude (2S)-1-(4-acetylpiperazin-1-yl)propan-2-ol (3.63 g, 100%) as a gum which was used without purification.

1H NMR (400.1 MHz, CDCl3); δ 1.14 (3H, d), 2.09 (3H, s), 2.25-2.43 (4H, m), 2.60-2.67 (2H, m), 3.22 (1H, s), 3.46-3.49 (2H, m), 3.56-3.70 (2H, m), 3.82-3.90 (1H, m).

### Example 27

### Preparation of 6-(4-{4-[(1S)-2-(4-acetylpiperazin-1-yl)-1-methylethoxy]phenyl}piperidin-1-yl)-3-(trifluoromethyl) [1,2,4]triazolo[4,3-b]pyridazine

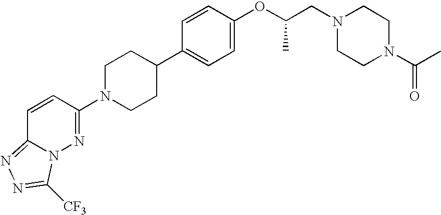

Obtained in 65% yield by an analogous method to Example 26, starting from 4-{1-[3-(trifluoromethyl)[1,2,4]triazolo[4,3-b]pyridazin-6-yl]piperidin-4-yl}phenol and (2R)-1-(4-acetylpiperazin-1-yl)propan-2-ol.

1H NMR (400.1 MHz, DMSO-d6); δ 1.22 (3H, d), 1.61-1.70 (2H, m), 1.86-1.90 (2H, m), 1.96 (3H, s), 2.38-2.48 (5H, m), 2.56-2.61 (1H, m), 2.77-2.83 (1H, m), 3.07-3.13 (2H, m), 3.37-3.39 (4H, m), 4.39-4.42 (2H, m), 4.55-4.62 (1H, m), 6.87 (2H, d), 7.16 (2H, d), 7.65 (1H, d), 8.23 (1H, d); m/z=532 [M+H]+.

The (2R)-1-(4-acetylpiperazin-1-yl)propan-2-ol used as starting material was obtained in 96% yield by an analogous method to Example 26, preparation of starting materials, starting from N-acetylpiperazine and (R)-2-methyloxirane.

1H NMR (400.1 MHz, CDCl3); δ 1.14 (3H, d), 2.09 (3H, s), 2.25-2.43 (4H, m), 2.60-2.67 (2H, m), 3.23 (1H, s), 3.46-3.49 (2H, m), 3.56-3.70 (2H, m), 3.83-3.89 (1H, m).

### Example 28

### Preparation of 6-(4-{4-[(1S)-2-(4-acetylpiperazin-1-yl)-1-(methoxymethyl)ethoxy]phenyl}piperidin-1-yl)-3-(trifluoromethyl)[1,2,4]triazolo[4,3-b]pyridazine

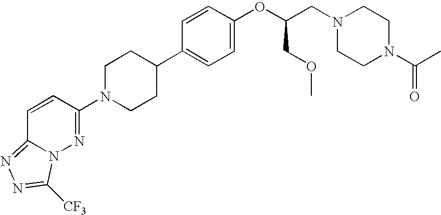

DIAD (625 mg, 2.48 mmol) in THF (5 mL) was added dropwise to 4-{1-[3-(trifluoromethyl)[1,2,4]triazolo[4,3-b]pyridazin-6-yl]piperidin-4-yl}phenol (obtained as described in Example 2, preparation of starting materials) (300 mg, 0.83 mmol), (2R)-1-(4-acetylpiperazin-1-yl)-3-methoxypropan-2-ol (536 mg, 2.48 mmol) and tri-n-butylphosphine (0.611 mL, 2.48 mmol) in THF (5 mL) at 0° C. under nitrogen. The resulting solution was stirred at 0° C. for 15 minutes and then at room temperature for 21 hours. Further portions of (2R)-1-(acetylpiperazin-1-yl)-3-methoxypropan-2-ol (180 mg, 0.83 mmol) in THF (1 mL), tri-n-butylphosphine (0.205 mL, 0.83 mmol) and DIAD (209 mg, 0.83 mmol) in THF (2 mL) were added and the mixture was stirred at room temperature for a further 72 hours. The reaction mixture was diluted with ethyl acetate (20 mL) and washed with saturated sodium bicarbonate (20 mL) and saturated brine (20 mL). The organic layer was dried (MgSO4), filtered and evaporated to a gum. The crude product was purified by flash silica chromatography, elution gradient 0 to 4% MeOH in DCM to afford 6-(4-{4-[(1S)-2-(4-acetylpiperazin-1-yl)-1-(methoxymethyl)ethoxy]phenyl}piperidin-1-yl)-3-(trifluoromethyl)[1,2,4]triazolo[4,3-b]pyridazine (91 mg, 19%) as a dry film.

1H NMR (400.1 MHz, DMSO-d6); δ 1.61-1.71 (2H, m), 1.87-1.90 (2H, m), 1.96 (3H, s), 2.37-2.47 (4H, m), 2.55-2.58 (2H, m), 2.77-2.83 (1H, m), 3.07-3.13 (2H, m), 3.27 (3H, s), 3.36-3.40 (4H, m), 3.48-3.57 (2H, m), 4.39-4.42 (2H, m), 4.53-4.58 (1H, m), 6.91 (2H, d), 7.16 (2H, d), 7.65 (1H, d), 8.24 (1H, d); m/z=562 [M+H]+.

The (2R)-1-(4-acetylpiperazin-1-yl)-3-methoxypropan-2-ol used as starting material was prepared as follows:—

(R)-2-(Methoxymethyl)oxirane (1.958 g, 22.23 mmol) was added to N-acetylpiperazine (2.59 g, 20.21 mmol) in MeOH (50 mL) and the solution was stirred at 80° C. for 2 hours. The solvent was evaporated to afford crude (2R)-1-(4-acetylpiperazin-1-yl)-3-methoxypropan-2-ol (4.14 g, 95%) as a gum which was used without purification.

1H NMR (400.1 MHz, CDCl3); δ 2.08 (3H, s), 2.37-2.53 (4H, m), 2.57-2.64 (2H, m), 3.12 (1H, s), 3.36-3.49 (4H, m), 3.39 (3H, s), 3.57-3.69 (2H, m), 3.87-3.93 (1H, m).

### Example 29

### Preparation of 6-(4-{4-[(1R)-2-(4-acetylpiperazin-1-yl)-1-(methoxymethyl)ethoxy]phenyl}piperidin-1-yl)-3-(trifluoromethyl)[1,2,4]triazolo[4,3-b]pyridazine

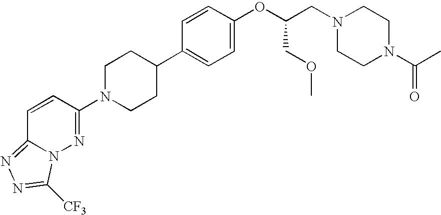

Obtained in 8% yield by an analogous method to Example 28, starting from 4-{1-[3-(trifluoromethyl)[1,2,4]triazolo[4,3-b]pyridazin-6-yl]piperidin-4-yl}phenol and (2S)-1-(4-acetylpiperazin-1-yl)-3-methoxypropan-2-ol.

1H NMR (400.1 MHz, CDCl3); δ 1.71-1.81 (2H, m), 1.98-2.01 (2H, m), 2.06 (3H, s), 2.48-2.54 (4H, m), 2.68-2.71 (2H, m), 2.74-2.81 (1H, m), 3.08-3.14 (2H, m), 3.38 (3H, s), 3.40-3.43 (2H, m), 3.56-3.59 (4H, m), 4.37 (2H, d), 4.47-4.49 (1H, m), 6.92 (2H, d), 7.11-7.13 (3H, m), 7.92 (1H, d); m/z=562 [M+H]+.

The (2S)-1-(4-acetylpiperazin-1-yl)-3-methoxypropan-2-ol used as starting material was obtained in 93% yield by an analogous method to Example 28, preparation of starting materials, starting from N-acetylpiperazine and (S)-2-(methoxymethyl)oxirane.

1H NMR (400.1 MHz, CDCl3); δ 2.08 (3H, s), 2.37-2.53 (4H, m), 2.57-2.64 (1H, m), 2.81-2.87 (1H, m), 3.13 (1H, s), 3.36-3.49 (4H, m), 3.39 (3H, s), 3.57-3.69 (2H, m), 3.87-3.93 (1H, m).

### Example 30

### Preparation of 4-{4-[2-(4-acetylpiperazin-1-yl)ethoxy]-2-methylphenyl}-1-[3-(trifluoromethyl)[1,2,4]-triazolo[4,3-b]pyridazin-6-yl]piperidin-4-ol

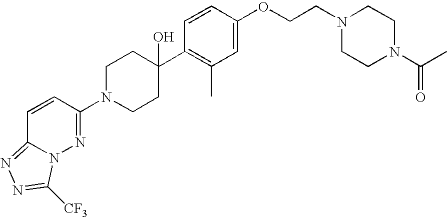

Methanesulfonyl chloride (0.451 mL, 5.81 mmol) was added dropwise to a solution of 2-(4-acetylpiperazine-1-yl)ethanol (obtained as described in PCT Int. Appl. WO2003064413, Example 28, preparation of starting materials) (1 g, 5.81 mmol) and triethylamine (0.897 mL, 6.39 mmol) in DCM (20 mL) at 0° C. under nitrogen. The resulting solution was stirred at 0° C. for 15 minutes, then allowed to warm to ambient temperature and stirred for a further 24 hours. The reaction mixture was washed with water (20 mL) and the organic layer was dried over MgSO4, filtered and evaporated. The resulting crude 2-(4-acetylpiperazin-1-yl)ethyl methanesulfonate (286 mg, 1.14 mmol) was dissolved in DMF (10 mL) and 4-(4-hydroxy-2-methylphenyl)-1-[3-(trifluoromethyl)[1,2,4]triazolo[4,3-b]pyridazin-6-yl]piperidin-4-ol (150 mg, 0.38 mmol) and potassium carbonate (264 mg, 1.91 mmol) were added. The resulting suspension was stirred under nitrogen at 100° C. for 3 hours. The reaction mixture was evaporated to dryness, redissolved in DCM (50 mL) and the solution was washed with water (4×50 mL) and saturated brine (50 mL). The organic layer was dried over MgSO4, filtered and evaporated to afford crude product. The crude product was purified by flash silica chromatography, elution gradient 0 to 20% MeOH in DCM containing 1% ammonia. Pure fractions were evaporated to dryness to afford 4-{4-[2-(4-acetylpiperazin-1-yl)ethoxy]-2-methylphenyl}-1-[3-(trifluoromethyl)[1,2,4]triazolo[4,3-b]pyridazin-6-yl]piperidin-4-ol (132 mg, 63%) as a white solid.

1H NMR (399.9 MHz, DMSO-d6) δ 1.91-2.06 (7H, m), 2.41 (2H, t), 2.45-2.56 (5H, m, obscured by DMSO), 2.70 (2H, t), 3.38-3.51 (6H, m), 4.05 (2H, t), 4.16 (2H, d), 5.01 (1H, s), 6.69 (1H, dd), 6.73 (1H, d), 7.26 (1H, d), 7.66 (1H, d), 8.25 (1H, d); m/z=548 [M+H]+.

The 4-(4-hydroxy-2-methylphenyl)-1-[3-(trifluoromethyl)[1,2,4]triazolo[4,3-b]pyridazin-6-yl]piperidin-4-ol used as starting material was prepared as follows:—

### Preparation of benzyl 4-[4-(benzyloxy)-2-methylphenyl]-4-hydroxypiperidine-1-carboxylate

Obtained in 38% yield by an analogous method to Example 3, preparation of starting materials, starting from benzyl 4-oxopiperidine-1-carboxylate and 1-(benzyloxy)-4-bromo-3-methylbenzene (CAS 17671-75-9).

1H NMR (399.9 MHz, DMSO-d6) δ 1.80-1.86 (4H, m), 2.49-2.54 (3H, m), 3.18-3.30 (2H, m), 3.91 (2H, d), 4.87 (1H, s), 5.09 (4H, d), 6.73-6.81 (2H, m), 7.23-7.47 (11H, m); m/z=430 [M−H]−.

### Preparation of 4-(4-hydroxy-2-methylphenyl)piperidin-4-ol

Obtained in 71% yield by an analogous method to Example 3, preparation of starting materials, starting from benzyl 4-[4-(benzyloxy)-2-methylphenyl]-4-hydroxypiperidine-1-carboxylate.

1H NMR (399.9 MHz, DMSO-d6) δ 1.62-1.91 (4H, m), 2.47 (3H, s), 2.73-2.85 (2H, m), 2.92-3.06 (2H, m), 4.37 (1H, br s), 4.56 (1H, br s), 6.46-6.55 (2H, m), 7.15 (1H, d), 8.41-9.67 (1H, m); m/z=208 [M+H]+.

### Preparation of 4-(4-hydroxy-2-methylphenyl)-1-[3-(trifluoromethyl)[1,2,4]triazolo[4,3-b]pyridazin-6-yl]piperidin-4-ol

Obtained in 71% yield by an analogous method to Example 3, preparation of starting materials, starting from 6-chloro-3-(trifluoromethyl)-[1,2,4]triazolo[4,3-b]pyridazine (obtained as described in Monatsh. Chem. 1972, 103, 1591) and 4-(4-hydroxy-2-methylphenyl)piperidin-4-ol.

1H NMR (399.9 MHz, DMSO-d6) δ 1.91-2.05 (4H, m), 2.48 (3H, s), 3.40-3.51 (2H, m), 4.14 (2H, d), 4.91 (1H, s), 6.51 (1H, dd), 6.55 (1H, d), 7.14 (1H, d), 7.64 (1H, d), 8.23 (1H, d), 9.12 (1H, s); m/z=394 [M+H]+.

### Example 31

### Preparation of 4-[2-(4-{4-hydroxy-1-[3-(trifluoromethyl)[1,2,4]-triazolo[4,3-b]pyridazin-6-yl]piperidin-4-yl}-3-methylphenoxy)ethyl]-1-methylpiperazin-2-one

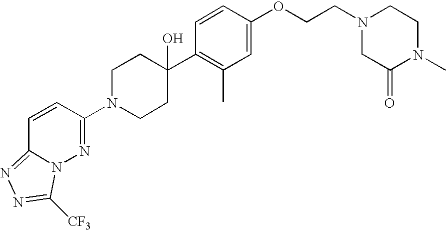

Methanesulfonyl chloride (0.491 mL, 6.32 mmol) was added dropwise to a solution of 4-(2-hydroxyethyl)-1-methylpiperazin-2-one (obtained as described in Example 3, preparation of starting materials) (1 g, 6.32 mmol) and triethylamine (0.976 mL, 6.95 mmol) in DCM (20 mL) at 0° C. under nitrogen. The resulting solution was stirred at 0° C. for 15 minutes, then allowed to warm to ambient temperature and stirred for a further 24 hours. The reaction mixture was washed with water (20 mL) and the organic layer was dried over MgSO4, filtered and evaporated to dryness. The resulting crude 2-(4-methyl-3-oxopiperazin-1-yl)ethyl methanesulfonate (270 mg, 1.14 mmol) was dissolved in DMF (10 mL) and 4-(4-hydroxy-2-methylphenyl)-1-[3-(trifluoromethyl)[1,2,4]triazolo[4,3-b]pyridazin-6-yl]piperidin-4-ol (obtained as described in Example 30, preparation of starting materials) (150 mg, 0.38 mmol) and potassium carbonate (264 mg, 1.91 mmol) were added. The resulting suspension was stirred under nitrogen at 100° C. for 3.5 hours. The reaction mixture was evaporated to dryness, redissolved in DCM (25 mL) and the solution was washed with water (4×50 mL). The aqueous washings were further extracted with DCM (25 mL). The combined organic layers were washed with saturated brine (50 mL), dried over MgSO4, filtered and evaporated to afford crude product. The crude product was purified by flash silica chromatography, elution gradient 0 to 20% MeOH in DCM containing 1% ammonia. Pure fractions were evaporated to dryness to afford 4-[2-(4-{4-hydroxy-1-[3-(trifluoromethyl)[1,2,4]triazolo[4,3-b]pyridazin-6-yl]piperidin-4-yl}-3-methylphenoxy)ethyl]-1-methylpiperazin-2-one (41 mg, 20%) as a yellow solid.

1H NMR (399.9 MHz, CDCl3) δ 2.05-2.11 (4H, m), 2.54 (3H, s), 2.77 (4H, q), 2.88 (4H, s), 3.20 (2H, s), 3.27 (2H, t), 3.50-3.60 (2H, m), 4.02 (2H, t), 4.04-4.11 (2H, m), 6.61 (1H, dd), 6.69 (1H, d), 7.08 (1H, d), 7.18 (1H, d, partially obscured by CHCl3), 7.85 (1H, d); m/z=534 [M+H]+.

### Example 32

### Preparation of 4-methyl-1-[2-(4-{1-[3-(trifluoromethyl)[1,2,4]-triazolo[4,3-b]pyridazin-6-yl]piperidin-4-yl}phenoxy)ethyl]-1,4-diazepan-5-one

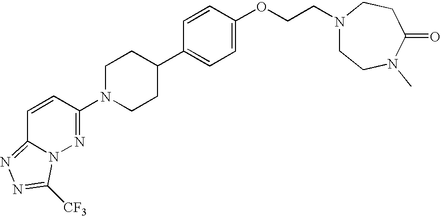

2-(4-{1-[3-(Trifluoromethyl)[1,2,4]triazolo[4,3-b]pyridazin-6-yl]piperidin-4-yl}phenoxy)ethyl methanesulfonate (obtained as described in Example 2, preparation of starting materials) (200 mg, 0.41 mmol), 4-methyl-1,4-diazepan-5-one hydrochloride (136 mg, 0.82 mmol), DIPEA (0.287 mL, 1.65 mmol) and sodium iodide (61.8 mg, 0.41 mmol) were suspended in DMA (2 mL) and sealed into a microwave tube. The reaction was heated to 150° C. for 2 hours in the microwave reactor and cooled to room temperature. Silica was added, the solvent was evaporated and the crude product was purified by flash silica chromatography, elution gradient 0 to 5% MeOH in DCM. Pure fractions were evaporated to dryness, then further purified by ion exchange chromatography using an SCX column. The desired product was eluted from the column using 2M ammonia/MeOH and the solvents were evaporated to dryness to afford a gum, which was triturated with ether. The resulting solid was collected by filtration and dried to give 4-methyl-1-[2-(4-{1-[3-(trifluoromethyl)[1,2,4]triazolo[4,3-b]pyridazin-6-yl]piperidin-4-yl}phenoxy)ethyl]-1,4-diazepan-5-one (53 mg, 25%) as a solid.

1H NMR (399.9 MHz, DMSO-d6) δ 1.66 (2H, m), 1.87 (2H, m), 2.59-2.68 (6H, m), 2.76-2.85 (6H, m), 3.09 (2H, m), 3.42 (2H, m), 4.04 (2H, t), 4.41 (2H, m), 6.87 (2H, d), 7.17 (2H, d), 7.67 (1H, d), 8.25 (1H, d); m/z=518 [M+H]+.

### Example 33

### Preparation of 6-[4-(4-{2-[(3S)-4-acetyl-3-methylpiperazin-1-yl]ethoxy}phenyl)piperidin-1-yl]-3-(trifluoromethyl)[1,2,4]triazolo[4,3-b]pyridazine

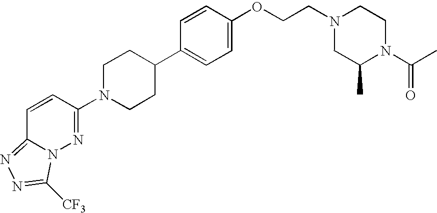

Obtained in 11% yield by an analogous method to Example 32, starting from 2-(4-{1-[3-(trifluoromethyl)[1,2,4]triazolo[4,3-b]pyridazin-6-yl]piperidin-4-yl}phenoxy)ethyl methanesulfonate (obtained as described in Example 2, preparation of starting materials) and (2S)-N-acetyl-2-methylpiperazine.

1H NMR (500.1 MHz, DMSO-d6 @ 373K) δ 1.19 (3H, d), 1.70 (2H, m), 1.91-1.96 (5H, m), 2.05 (1H, m), 2.22 (1H, m), 2.65-2.76 (4H, m), 2.83 (1H, m), 3.03 (1H, m), 3.15 (2H, m), 3.86 (1H, m), 4.08 (2H, t), 4.27 (1H, m), 4.36 (2H, m), 6.87 (2H, d), 7.16 (2H, d), 7.53 (1H, d), 8.12 (1H, d); m/z=532 [M+H]+.

### Example 34

### Preparation of 6-[4-(4-{2-[(3R)-4-acetyl-3-methylpiperazin-1-yl]ethoxy}phenyl)piperidin-1-yl]-3-(trifluoromethyl)[1,2,4]triazolo[4,3-b]pyridazine

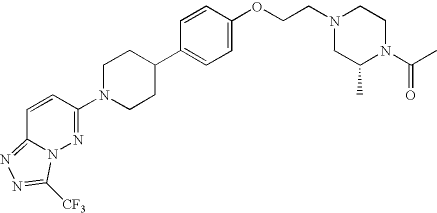

Obtained in 48% yield by an analogous method to Example 32, starting from 2-(4-{1-[3-(trifluoromethyl)[1,2,4]triazolo[4,3-b]pyridazin-6-yl]piperidin-4-yl}phenoxy)ethyl methanesulfonate (obtained as described in Example 2, preparation of starting materials) and (2R)-N-acetyl-2-methylpiperazine.

1H NMR (500.1 MHz, DMSO-d6 @ 373K) δ 1.19 (3H, d), 1.70 (2H, m), 1.91-1.96 (5H, m), 2.06 (1H, m), 2.23 (1H, m), 2.67-2.77 (4H, m), 2.83 (1H, m), 3.04 (1H, m), 3.15 (2H, m), 3.86 (1H, m), 4.08 (2H, t), 4.27 (1H, m), 4.36 (2H, m), 6.88 (2H, d), 7.16 (2H, d), 7.53 (1H, d), 8.12 (1H, d); m/z=532 [M+H]+.

### Example 35

### Preparation of 4-{4-[2-(4-acetylpiperazin-1-yl)ethoxy]-2-fluorophenyl}-1-[3-(trifluoromethyl)[1,2,4]-triazolo[4,3-b]pyridazin-6-yl]piperidin-4-ol

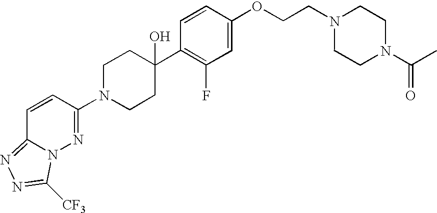

Obtained in 62% yield by an analogous method to Example 30, starting from 4-(2-fluoro-4-hydroxyphenyl)-1-[3-(trifluoromethyl)[1,2,4]triazolo[4,3-b]pyridazin-6-yl]piperidin-4-ol and 2-(4-acetylpiperazine-1-yl)ethanol.

1H NMR (399.9 MHz, DMSO-d6) δ 1.72 (2H, d), 1.99 (3H, s), 2.13-2.24 (2H, m), 2.41 (2H, t), 2.48 (2H, t), 2.71 (2H, t), 3.37-3.48 (6H, m), 4.09 (2H, t), 4.19 (2H, d), 5.41 (1H, s), 6.73-6.82 (2H, m), 7.54 (1H, t), 7.68 (1H, d), 8.26 (1H, d); m/z=552 [M+H]. The 4-(2-fluoro-4-hydroxyphenyl)-1-[3-(trifluoromethyl)[1,2,4]triazolo[4,3-b]pyridazin-6-yl]piperidin-4-ol used as starting material was prepared as follows:—

### Preparation of benzyl 4-[4-(benzyloxy)-2-fluorophenyl]-4-hydroxypiperidine-1-carboxylate

Obtained in 61% yield by an analogous method to Example 3, preparation of starting materials, starting from benzyl 4-oxopiperidine-1-carboxylate and 1-(benzyloxy)-4-bromo-3-fluorobenzene (CAS 185346-79-6).

1H NMR (399.9 MHz, DMSO-d6) δ 1.58 (2H, d), 1.95-2.09 (2H, m), 3.11-3.30 (2H, m), 3.92 (2H, d), 5.11 (2H, s), 5.13 (2H, s), 5.31 (1H, s), 6.80-6.88 (2H, m), 7.31-7.47 (10H, m), 7.52 (1H, t); m/z=434 [M−H]−.

### Preparation of 4-(2-fluoro-4-hydroxyphenyl)piperidin-4-ol

Obtained in 100% yield by an analogous method to Example 3, preparation of starting materials, starting from benzyl 4-[4-(benzyloxy)-2-fluorophenyl]-4-hydroxypiperidine-1-carboxylate.

1H NMR (399.9 MHz, DMSO-d6) δ 1.47 (2H, d), 1.94-2.05 (2H, m), 2.71 (2H, d), 2.93 (2H, t, partially obscured by H2O), 4.86 (1H, br s), 6.47 (1H, dd), 6.55 (1H, dd), 7.37 (1H, dd), 8.51 (1H, s).

### Preparation of 4-(2-fluoro-4-hydroxyphenyl)-1-[3-(trifluoromethyl)[1,2,4]triazolo[4,3-b]pyridazin-6-yl]piperidin-4-ol

Obtained in 80% yield by an analogous method to Example 3, preparation of starting materials, starting from 6-chloro-3-(trifluoromethyl)-[1,2,4]triazolo[4,3-b]pyridazine and 4-(2-fluoro-4-hydroxyphenyl)piperidin-4-ol.

1H NMR (399.9 MHz, DMSO-d6) δ 1.71 (2H, d), 2.10-2.22 (2H, m), 3.31-3.46 (2H, m, obscured by H2O), 4.16 (2H, d), 5.33 (1H, s), 6.47 (1H, dd), 6.60 (1H, dd), 7.42 (1H, dd), 7.66 (1H, d), 8.25 (1H, d), 9.77 (1H, s); m/z=398 [M+H]+.

### Example 36

### Preparation of 4-[2-(3-fluoro-4-[4-hydroxy-1-[3-(trifluoromethyl)[1,2,4]triazolo[4,3-b]pyridazin-6-yl]piperidin-4-yl]phenoxy)ethyl]-1-methylpiperazin-2-one

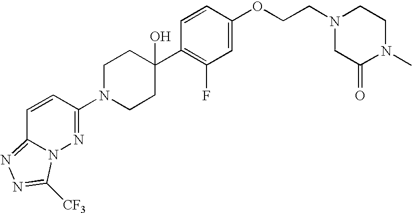

Obtained in 31% yield by an analogous method to Example 31, starting from 4-(2-fluoro-4-hydroxyphenyl)-1-[3-(trifluoromethyl)[1,2,4]triazolo[4,3-b]pyridazin-6-yl]piperidin-4-ol (obtained as described in Example 35, preparation of starting materials) and 4-(2-hydroxyethyl)-1-methylpiperazin-2-one.

1H NMR (399.9 MHz, DMSO-d6) δ 1.72 (2H, d), 2.12-2.24 (2H, m), 2.75 (4H, t), 2.82 (3H, s), 3.09 (2H, s), 3.26 (2H, t), 3.42 (2H, t), 4.09 (2H, t), 4.19 (2H, d), 5.42 (1H, s), 6.73-6.83 (2H, m), 7.54 (1H, t), 7.68 (1H, d), 8.26 (1H, d); m/z=538 [M+H]+.

### Examples 37-40

The following compounds were prepared in 38-51% yield by an analogous method to Example 30, starting from 4-(4-hydroxyphenyl)-1-[3-(trifluoromethyl)[1,2,4]triazolo[4,3-b]pyridazin-6-yl]piperidin-4-ol (obtained as described in Example 3, preparation of starting materials) and the appropriate alcohol. The alcohol was prepared from the appropriate piperazinone by an analogous method to that used to prepare 4-(2-hydroxyethyl)-1-methylpiperazin-2-one in Example 3.

For example in Example 37, the alcohol used was

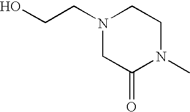

1H NMR: (400.132 MHz, CDCl3) δ 1.08 (3H, t), 2.54 (2H, t), 2.71 (2H, t), 3.14 (2H, s), 3.27 (2H, t), 3.37 (2H, q), 3.60 (2H, t)

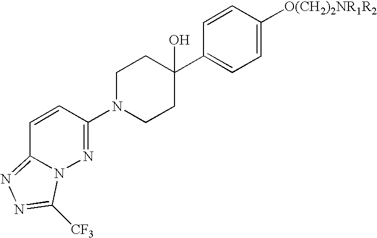

| Ex. | NR₁R₂ | 1H NMR | m/z [M + H]+ |
|---|---|---|---|
| 37 | 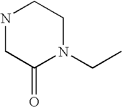 | δ (399.9 MHz, CDCl3) 1.07 (3H, t), 1.87 (2H, m), 2.06 (2H, m), 2.79 (4H, m), 3.20-3.42 (6H, m), 3.52 (2H, m), 4.06 (4H, m), 6.84 (2H, d), 7.07 (1H, d), 7.33 (2H, d), 7.85 (1H, d) | 535 |
| 38 | 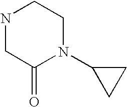 | δ (399.9 MHz, CDCl3) 0.59 (2H, m), 0.74 (2H, m), 1.87 (2H, m), 2.05 (2H, m), 2.64 (1H, m), 2.76 (4H, m), 3.21 (4H, m), 3.52 (2H, m), 4.06 (4H, m), 6.83 (2H, d), 7.06 (1H, d), 7.33 (2H, d), 7.85 (1H, d) | 547 |
| 39 | 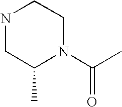 | δ (499.8 MHz, DMSO-d6) 1.17 (3H, d), 1.96- 2.08 (6H, m), 2.19-2.24 (1H, m), 2.63-2.78 (3H, m), 3.49 (2H, t), 3.86 (1H, bs), 4.07-4.16 (4H, m), 4.26 (1H, bs), 4.76 (1H, s), 6.88 (2H, d), 7.40 (2H, d), 7.54 (1H, d), 8.12 (1H, d) | 549 |
| 40 | 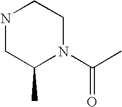 | δ (499.8 MHz, DMSO-d6) 1.17 (3H, d), 1.96- 2.08 (6H, m), 2.19-2.24 (1H, m), 2.63-2.78 (3H, m), 3.49 (2H, t), 3.86 (1H, bs), 4.07-4.16 (4H, m), 4.26 (1H, bs), 4.76 (1H, s), 6.88 (2H, d), 7.40 (2H, d), 7.54 (1H, d), 8.12 | 549 |

### Examples 41-42

The following compounds were prepared in 62-74% yield by an analogous method to Example 30, starting from 4-(2-fluoro-4-hydroxyphenyl)-1-[3-(trifluoromethyl)[1,2,4]triazolo[4,3-b]pyridazin-6-yl]piperidin-4-ol (obtained as described in Example 35, preparation of starting materials) and the appropriate alcohol. The alcohol was prepared from the appropriate piperazinone by an analogous method to that used to prepare 4-(2-hydroxyethyl)-1-methylpiperazin-2-one in Example 3.

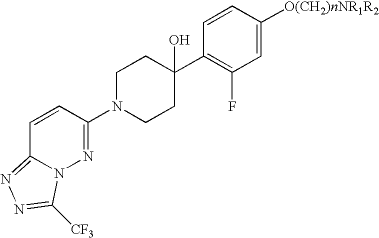

| Ex. | n | NR₁R₂ | 1H NMR (399.9 MHz, DMSO-d6) | m/z [M + H]+ |
|---|---|---|---|---|
| 41 | 2 | 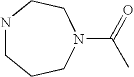 | δ 1.64-1.85 (4H, m), 1.99 (3H, s), 2.13-2.26 (2H, m), 2.59-2.90 (6H, m), 3.35-3.52 (6H, m), 3.99- 4.07 (2H, m), 4.16 (2H, d), 5.43 (1H, s), 6.69- 6.82 (2H, m), 7.53 (1H, t), 7.67 (1H, d), 8.26 (1H, d) | 567 |
| 42 | 3 | 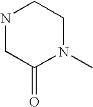 | δ 1.73 (2H, d), 1.87 (2H, t), 2.19 (2H, td), 2.47 (2H, t, partially obscured by solvent), 2.65 (2H, t), 2.82 (3H, s), 2.98 (2H, s), 3.23-3.29 (2H, m), 3.43 (2H, t), 4.00 (2H, t), 4.18 (2H, d), 5.37 (1H, s), 6.72 (1H, dd), 6.78 (1H, dd), 7.54 (1H, t), 7.67 (1H, d), 8.25 (1H, d). | 553 |

### Examples 43 and 44

### Preparation of (R)-4-{4-[2-(4-acetylpiperazin-1-yl)propoxy]phenyl}-1-[3-(trifluoromethyl)[1,2,4]triazolo[4,3-b]pyridazin-6-yl]piperidin-4-ol and (S)-4-{4-[2-(4-acetylpiperazin-1-yl)propoxy]phenyl}-1-[3-(trifluoromethyl)[1,2,4]-triazolo[4,3-b]pyridazin-6-yl]piperidin-4-ol

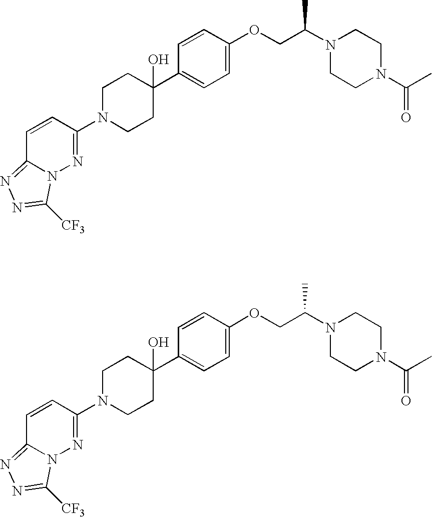

Acetic acid (0.091 mL, 1.58 mmol) was added to 1-(4-{4-hydroxy-1-[3-(trifluoromethyl)[1,2,4]triazolo[4,3-b]pyridazin-6-yl]piperidin-4-yl}phenoxy)propan-2-one (690 mg, 1.58 mmol), N-acetylpiperazine (305 mg, 2.38 mmol) and a catalytic amount of MgSO4 in THF (10 mL). The resulting mixture was stirred at ambient temperature for 4 hours then sodium triacetoxyhydroborate (403 mg, 1.90 mmol) was added and stirring continued for a further 16 hours. The reaction mixture was concentrated and diluted with DCM (25 mL), washed with saturated NaHCO3 (25 mL) and then filtered through a PTFE cup. The organic layer was evaporated to afford crude product. The crude product was purified by flash silica chromatography, elution gradient 0 to 5% MeOH in DCM. Pure fractions were evaporated to dryness to afford racemic 4-{4-[2-(4-acetylpiperazin-1-yl)propoxy]phenyl}-1-[3-(trifluoromethyl)[1,2,4]triazolo[4,3-b]pyridazin-6-yl]piperidin-4-ol (170 mg, 19.6%) as a solid. 166 mg of the racemic product was purified by preparative chiral-HPLC on a Merck 50 mm 20 μm Chiralpak AS column, eluting isocratically with MeOH eluent. The fractions containing the desired compound were evaporated to dryness to afford the first eluting enantiomer of 4-{4-[2-(4-acetylpiperazin-1-yl)propoxy]phenyl}-1-[3-(trifluoromethyl)[1,2,4]triazolo[4,3-b]pyridazin-6-yl]piperidin-4-ol (65.1 mg).

1H NMR (399.9 MHz, DMSO-d6) δ 1.07 (3H, d), 1.71 (2H, s), 1.94-2.02 (5H, m), 2.45-2.63 (4H, m, partially obscured by DMSO peak), 2.99 (1H, q), 3.30-3.45 (6H, partially obscured water peak, m), 3.83-3.87 (1H, m), 4.01-4.05 (1H, m), 4.19 (2H, d), 5.14 (1H, s), 6.89 (2H, s), 7.38-7.41 (2H, m), 7.67 (1H, d), 8.25 (1H, d); m/z=548 [M+H]+.

Further elution from the chiral chromatography column gave the second eluting enantiomer of 4-{4-[2-(4-acetylpiperazin-1-yl)propoxy]phenyl}-1-[3-(trifluoromethyl)[1,2,4]triazolo[4,3-b]pyridazin-6-yl]piperidin-4-ol (60 mg).

1H NMR (399.9 MHz, DMSO-d6) δ 1.07 (3H, d), 1.71 (2H, s), 1.94-2.02 (5H, m), 2.45-2.63 (4H, m, partially obscured by DMSO peak), 2.99 (1H, q), 3.30-3.45 (6H, partially obscured water peak, m), 3.83-3.87 (1H, m), 4.01-4.05 (1H, m), 4.19 (2H, d), 5.14 (1H, s), 6.89 (2H, s), 7.38-7.41 (2H, m), 7.67 (1H, d), 8.25 (1H, d); m/z=548 [M+H]+. The 1-(4-{4-hydroxy-1-[3-(trifluoromethyl)[1,2,4]triazolo[4,3-b]pyridazin-6-yl]piperidin-4-yl}phenoxy)propan-2-one used as starting material was prepared as follows:—

Potassium carbonate (820 mg, 5.93 mmol) was added to 4-(4-hydroxyphenyl)-1-[3-(trifluoromethyl)[1,2,4]triazolo[4,3-b]pyridazin-6-yl]piperidin-4-ol (obtained as described in Example 3, preparation of starting materials) (750 mg, 1.98 mmol) and 1-chloropropan-2-one (0.315 mL, 3.95 mmol) in DMA (10 mL). The resulting mixture was stirred at 100° C. for 2 hours. Further potassium carbonate (820 mg, 5.93 mmol) and 1-chloropropan-2-one (0.315 mL, 3.95 mmol) were added and the mixture was stirred at 100° C. for a further 2 hours. The reaction mixture was cooled to room temperature, concentrated and diluted with water (25 mL) then extracted with DCM (2×25 mL). The organic layer was dried over MgSO4, filtered and evaporated to afford crude product. The crude product was purified by flash silica chromatography, elution gradient 50 to 100% EtOAc in isohexane. Pure fractions were evaporated to dryness to afford 1-(4-{4-hydroxy-1-[3-(trifluoromethyl)[1,2,4]triazolo[4,3-b]pyridazin-6-yl]piperidin-4-yl}phenoxy)propan-2-on (692 mg, 80%) as a gum.

1H NMR (399.9 MHz, CDCl3) δ 1.86 (2H, m), 2.05 (2H, m), 2.20 (3H, s), 3.52 (2H, m), 4.08 (2H, m), 4.48 (2H, s), 6.81 (2H, d), 7.07 (1H, d), 7.35 (2H, d), 7.85 (1H, d).

### Examples 45 and 46

### Preparation of (R)-4-{4-[2-(4-acetyl-1,4-diazepan-1-yl)propoxy]phenyl}-1-[3-(trifluoromethyl)[1,2,4]triazolo[4,3-b]pyridazin-6-yl]piperidin-4-ol and (S)-4-{4-[2-(4-acetyl-1,4-diazepan-1-yl)propoxy]phenyl}-1-[3-(trifluoromethyl)[1,2,4]-triazolo[4,3-h]pyridazin-6-yl]piperidin-4-ol

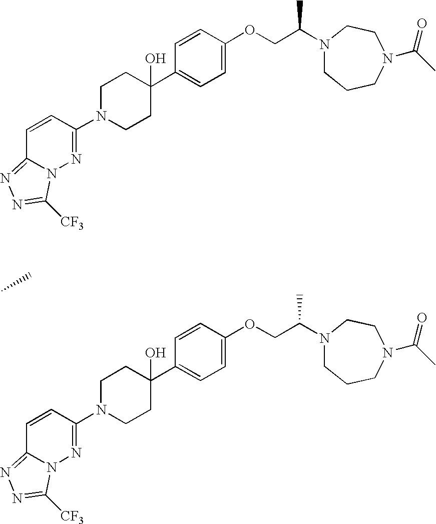

Racemic 4-{4-[2-(4-acetyl-1,4-diazepan-1-yl)propoxy]phenyl}-1-[3-(trifluoromethyl)[1,2,4]triazolo[4,3-b]pyridazin-6-yl]piperidin-4-ol was obtained in 54% yield by an analogous method to Examples 43 and 44, starting from 1-(4-{4-hydroxy-1-[3-(trifluoromethyl)[1,2,4]triazolo[4,3-b]pyridazin-6-yl]piperidin-4-yl}phenoxy)propan-2-one and N-acetylhomopiperazine. The racemic product was purified by preparative chiral-SFC on a Merck 50 mm 20 μm Chiralpak AD-HSFC, eluting isocratically with 70/30 CO₂/IPA. Fractions containing the desired compound were evaporated to dryness to afford the first eluting enantiomer (76% recovery from racemate).

1H NMR (399.9 MHz, DMSO-d6) δ 1.04-1.07 (3H, m), 1.58-1.64 (1H, m), 1.69-1.73 (3H, m), 1.94-2.02 (5H, m), 2.59-2.74 (3H, m), 2.75-2.82 (1H, m), 3.07-3.14 (1H, m), 3.38-3.46 (6H, m), 3.79-3.84 (1H, m), 3.96-4.01 (1H, m), 4.19 (2H, d), 5.14 (1H, s), 6.86-6.89 (2H, m), 7.39 (2H, d), 7.67 (1H, d), 8.24 (1H, d); m/z=562 [M+H]+.

Further elution from the chiral chromatography column gave the second eluting enantiomer (71% recovery from racemate).

1H NMR (399.9 MHz, DMSO-d6) δ 1.04-1.07 (3H, m), 1.58-1.64 (1H, m), 1.69-1.73 (3H, m), 1.94-2.02 (5H, m), 2.59-2.74 (3H, m), 2.75-2.82 (1H, m), 3.07-3.14 (1H, m), 3.38-3.46 (6H, m), 3.79-3.84 (1H, m), 3.96-4.01 (1H, m), 4.19 (2H, d), 5.14 (1H, s), 6.86-6.89 (2H, m), 7.39 (2H, d), 7.67 (1H, d), 8.24 (1H, d); m/z=562 [M+H]+.

### LIST OF FIGURES

Figure A: X-Ray Powder Diffraction Pattern for 6-(4-{4-[3-(4-acetylpiperazin-1-yl)propoxy]phenyl}piperazin-1-yl)-3-(trifluoromethyl)[1,2,4]triazolo[4,3-b]pyridazine Form A when measured using CuKa radiation.

Figure B: DSC Thermogram of 6-(4-{4-[3-(4-acetylpiperazin-1-yl)propoxy]phenyl}piperazin-1-yl)-3-(trifluoromethyl)[1,2,4]triazolo[4,3-b]pyridazine Form A

1. A compound of Formula (I), or a pharmaceutically acceptable salt thereof: 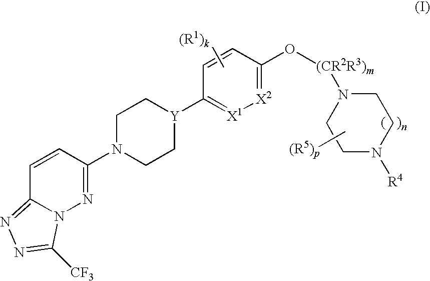
wherein
X¹-X² represents CH—CH, N—CH or CH—N;
Y represents N, CH or COH;
R¹, identically or differently on each occurrence, represents halo or C₁₋₆alkyl;
R² and R³, identically or differently on each occurrence, represent hydrogen, methyl, ethyl, isopropyl, cyclopropyl or methoxymethyl;
R⁴ represents C₁₋₆alkyl, C₂₋₆alkanoyl, C₁₋₆alkoxyC₁₋₆alkyl, C₃₋₆cycloalkyl, hydroxyC₂₋₆alkanoyl, C₁₋₆alkoxyC₂₋₆alkanoyl or oxetan-3-ylcarbonyl;
R⁵ represents oxo, methyl, ethyl, isopropyl, cyclopropyl or methoxymethyl;
is k represents 0, 1 or 2;
m represents 1, 2, 3 or 4;
n represents 1 or 2; and
p represents 0, 1 or 2;
with the proviso that the compound of Formula (I) is other than:
6-(4-{4-[3-(4-methylpiperazin-1-yl)propoxy]phenyl}piperidin-1-yl)-3-(trifluoromethyl)[1,2,4]triazolo[4,3-b]pyridazine;
6-(4-{4-[2-(4-methylpiperazin-1-yl)ethoxy]phenyl}piperidin-1-yl)-3-(trifluoromethyl)[1,2,4]triazolo[4,3-b]pyridazine;
6-[4-[4-[2-(4-acetylpiperazin-1-yl)ethoxy]phenyl]piperidin-1-yl]-3-(trifluoromethyl)[1,2,4]triazolo[4,3-b]pyridazine;
6-[4-[4-[3-(4-acetylpiperazin-1-yl)propoxy]phenyl]piperidin-1-yl]-3-(trifluoromethyl)[1,2,4]triazolo[4,3-b]pyridazine; or
4-[4-[2-(4-acetylpiperazin-1-yl)ethoxy]phenyl]-1-[3-(trifluoromethyl)[1,2,4]triazolo[4,3-b]pyridazin-6-yl]piperidin-4-ol.

2. A compound of formula (I) or a pharmaceutically acceptable salt thereof 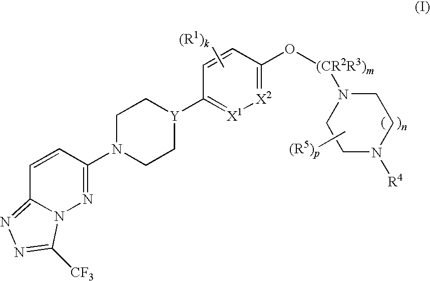
wherein
X¹-X² represents CH—CH, N—CH or CH—N;
Y represents N, CH or COH;
is R¹, identically or differently on each occurrence, represents halo or C₁₋₆alkyl;
R² and R³, identically or differently on each occurrence, represent hydrogen, methyl, ethyl, isopropyl, cyclopropyl or methoxymethyl;
R⁴ represents C₁₋₆alkyl, C₂₋₆alkanoyl, C₁₋₆alkoxyC₁₋₆alkyl, C₃₋₆cycloalkyl, hydroxyC₂₋₆alkanoyl, C₁₋₆alkoxyC₂₋₆alkanoyl or oxetan-3-ylcarbonyl;
R⁵ represents oxo, methyl, ethyl, isopropyl, cyclopropyl or methoxymethyl;
k represents 0, 1 or 2;
m represents 1, 2, 3 or 4;
n represents 1 or 2; and
p represents 1 or 2.

3. A compound according to claim 1 or claim 2, or a pharmaceutically acceptable salt thereof, wherein X¹-X² represents CH—CH.

4. A compound according to claim 1 or claim 2, or a pharmaceutically acceptable salt thereof, wherein Y represents N.

5. A compound according to claim 1, or a pharmaceutically acceptable salt thereof, wherein k represents 0, m represents 2 or 3, n represents 1 and p represents 0 or 1.

6. A compound according to claim 2, or a pharmaceutically acceptable salt thereof, wherein k represents 0, m represents 2 or 3, n represents 1 and p represents 1.

7. A compound according to claim 1 or claim 2, or a pharmaceutically acceptable salt thereof, wherein R² and R³ both represent hydrogen.

8. A compound according to claim 1 or claim 2, or a pharmaceutically acceptable salt thereof, wherein R⁴ represents methyl or acetyl.

9. A compound according to claim 1 selected from:
6-(4-{4-[3-(4-acetylpiperazin-1-yl)propoxy]phenyl}piperazin-1-yl)-3-(trifluoromethyl)[1,2,4]triazolo[4,3-b]pyridazine;
1-methyl-4-[2-(4-{1-[3-(trifluoromethyl)[1,2,4]triazolo[4,3-b]pyridazin-6-yl]piperidin-4-yl}phenoxy)ethyl]piperazin-2-one;
4-[2-(4-{4-hydroxy-1-[3-(trifluoromethyl)[1,2,4]triazolo[4,3-b]pyridazin-6-yl]piperidin-4-yl}phenoxy)ethyl]-1-methylpiperazin-2-one;
1-methyl-4-[2-(4-{4-[3-(trifluoromethyl)[1,2,4]triazolo[4,3-b]pyridazin-6-yl]piperazin-1-yl}phenoxy)ethyl]piperazin-2-one;
1-cyclopropyl-4-[2-(4-{4-[3-(trifluoromethyl)[1,2,4]triazolo[4,3-b]pyridazin-6-yl]piperazin-1-yl}phenoxy)ethyl]piperazin-2-one;
1-cyclopropyl-4-[2-(4-{1-[3-(trifluoromethyl)[1,2,4]triazolo[4,3-b]pyridazin-6-yl]piperidin-4-yl}phenoxy)ethyl]piperazin-2-one;
4-methyl-1-[2-(4-{4-[3-(trifluoromethyl)[1,2,4]triazolo[4,3-b]pyridazin-6-yl]piperazin-1-yl}phenoxy)ethyl]piperazin-2-one;
4-methyl-1-[2-(4-{1-[3-(trifluoromethyl)[1,2,4]triazolo[4,3-b]pyridazin-6-yl]piperidin-4-yl}phenoxy)ethyl]piperazin-2-one;
1-methyl-4-[3-(4-{1-[3-(trifluoromethyl)[1,2,4]triazolo[4,3-b]pyridazin-6-yl]piperidin-4-yl}phenoxy)propyl]piperazin-2-one;
1-cyclopropyl-4-[3-(4-{1-[3-(trifluoromethyl)[1,2,4]triazolo[4,3-b]pyridazin-6-yl]piperidin-4-yl}phenoxy)propyl]piperazin-2-one;
1-methyl-4-{2-[(5-{1-[3-(trifluoromethyl)[1,2,4]triazolo[4,3-b]pyridazin-6-yl]piperidin-4-yl}pyridin-2-yl)oxy]ethyl}piperazin-2-one;
4-[2-(2-fluoro-4-{4-hydroxy-1-[3-(trifluoromethyl)[1,2,4]triazolo[4,3-b]pyridazin-6-yl]piperidin-4-yl}phenoxy)ethyl]-1-methylpiperazin-2-one;
4-[2-(2-fluoro-4-{1-[3-(trifluoromethyl)[1,2,4]triazolo[4,3-b]pyridazin-6-yl]piperidin-4-yl}phenoxy)ethyl]-1-methylpiperazin-2-one;
6-(4-{-4-[2-(4-acetyl-1,4-diazepan-1-yl)ethoxy]phenyl}piperidin-1-yl)-3-(trifluoromethyl)[1,2,4]triazolo[4,3-b]pyridazine;
1-ethyl-4-[3-(4-{1-[3-(trifluoromethyl)[1,2,4]triazolo[4,3-b]pyridazin-6-yl]piperidin-4-yl}phenoxy)propyl]piperazin-2-one;
1-(1-methylethyl)-4-[3-(4-{1-[3-(trifluoromethyl)[1,2,4]triazolo[4,3-b]pyridazin-6-yl]piperidin-4-yl}phenoxy)propyl]piperazin-2-one;
1-(2-methoxyethyl)-4-[3-(4-{1-[3-(trifluoromethyl)[1,2,4]triazolo[4,3-b]pyridazin-6-yl]piperidin-4-yl}phenoxy)propyl]piperazin-2-one;
1-ethyl-4-[2-(4-{1-[3-(trifluoromethyl)[1,2,4]triazolo[4,3-b]pyridazin-6-yl]piperidin-4-yl}phenoxy)ethyl]piperazin-2-one;
1-(1-methylethyl)-4-[2-(4-{1-[3-(trifluoromethyl)[1,2,4]triazolo[4,3-b]pyridazin-6-yl]piperidin-4-yl}phenoxy)ethyl]piperazin-2-one;
1-(2-methoxyethyl)-4-[2-(4-{1-[3-(trifluoromethyl)[1,2,4]triazolo[4,3-b]pyridazin-6-yl]piperidin-4-yl}phenoxy)ethyl]piperazin-2-one;
2-oxo-2-{4-[2-(4-{1-[3-(trifluoromethyl)[1,2,4]triazolo[4,3-b]pyridazin-6-yl]piperidin-4-yl}phenoxy)ethyl]piperazin-1-yl}ethanol;
6-[4-(4-{2-[4-(methoxyacetyl)piperazin-1-yl]ethoxy}phenyl)piperidin-1-yl]-3-(trifluoromethyl)[1,2,4]triazolo[4,3-b]pyridazine;
(2S)-1-oxo-1-{4-[2-(4-{1-[3-(trifluoromethyl)[1,2,4]triazolo[4,3-b]pyridazin-6-yl]piperidin-4-yl}phenoxy)ethyl]piperazin-1-yl}propan-2-ol;
(2R)-1-oxo-1-{4-[2-(4-{1-[3-(trifluoromethyl)[1,2,4]triazolo[4,3-b]pyridazin-6-yl]piperidin-4-yl}phenoxy)ethyl]piperazin-1-yl}propan-2-ol;
6-{4-[4-(2-{4-[(2S)-2-methoxypropanoyl]piperazin-1-yl}ethoxy)phenyl]piperidin-1-yl}-3-(trifluoromethyl)[1,2,4]triazolo[4,3-b]pyridazine;
6-(4-{4-[(1R)-2-(4-acetylpiperazin-1-yl)-1-methylethoxy]phenyl}piperidin-1-yl)-3-(trifluoromethyl)[1,2,4]triazolo[4,3-b]pyridazine;
6-(4-{4-[(1S)-2-(4-acetylpiperazin-1-yl)-1-methylethoxy]phenyl}piperidin-1-yl)-3-(trifluoromethyl)[1,2,4]triazolo[4,3-b]pyridazine;
is 6-(4-{4-[(1S)-2-(4-acetylpiperazin-1-yl)-1-(methoxymethyl)ethoxy]phenyl}piperidin-1-yl)-3-(trifluoromethyl)[1,2,4]triazolo[4,3-b]pyridazine;
6-(4-{4-[(1R)-2-(4-acetylpiperazin-1-yl)-1-(methoxymethyl)ethoxy]phenyl}piperidin-1-yl)-3-(trifluoromethyl)[1,2,4]triazolo[4,3-b]pyridazine;
4-{4-[2-(4-acetylpiperazin-1-yl)ethoxy]-2-methylphenyl}-1-[3-(trifluoromethyl)[1,2,4]triazolo[4,3-b]pyridazin-6-yl]piperidin-4-ol;
4-[2-(4-{4-hydroxy-1-[3-(trifluoromethyl)[1,2,4]triazolo[4,3-b]pyridazin-6-yl]piperidin-4-yl}-3-methylphenoxy)ethyl]-1-methylpiperazin-2-one;
4-methyl-1-[2-(4-{1-[3-(trifluoromethyl)[1,2,4]triazolo[4,3-b]pyridazin-6-yl]piperidin-4-yl}phenoxy)ethyl]-1,4-diazepan-5-one;
6-[4-(4-{2-[(3S)-4-acetyl-3-methylpiperazin-1-yl]ethoxy}phenyl)piperidin-1-yl]-3-(trifluoromethyl)[1,2,4]triazolo[4,3-b]pyridazine;
6-[4-(4-{2-[(3R)-4-acetyl-3-methylpiperazin-1-yl]ethoxy}phenyl)piperidin-1-yl]-3-(trifluoromethyl)[1,2,4]triazolo[4,3-b]pyridazine;
4-{4-[2-(4-acetylpiperazin-1-yl)ethoxy]-2-fluorophenyl}-1-[3-(trifluoromethyl)[1,2,4]triazolo[4,3-b]pyridazin-6-yl]piperidin-4-ol;
4-[2-(3-fluoro-4-{4-hydroxy-1-[3-(trifluoromethyl)[1,2,4]triazolo[4,3-b]pyridazin-6-yl]piperidin-4-yl}phenoxy)ethyl]-1-methylpiperazin-2-one;
1-ethyl-4-[2-(4-{4-hydroxy-1-[3-(trifluoromethyl)[1,2,4]triazolo[4,3-b]pyridazin-6-yl]piperidin-4-yl}phenoxy)ethyl]piperazin-2-one;
1-cyclopropyl-4-[2-(4-{4-hydroxy-1-[3-(trifluoromethyl)[1,2,4]triazolo[4,3-b]pyridazin-6-yl]piperidin-4-yl}phenoxy)ethyl]piperazin-2-one;
4-(4-{2-[(3R)-4-acetyl-3-methylpiperazin-1-yl]ethoxy}phenyl)-1-[3-(trifluoromethyl) [1,2,4]triazolo[4,3-b]pyridazin-6-yl]piperidin-4-ol;
4-(4-{2-[(3S)-4-acetyl-3-methylpiperazin-1-yl]ethoxy}phenyl)-1-[3-(trifluoromethyl) [1,2,4]triazolo[4,3-b]pyridazin-6-yl]piperidin-4-ol;
4-{4-[2-(4-acetyl-1,4-diazepan-1-yl)ethoxy]-2-fluorophenyl}-1-[3-(trifluoromethyl) [1,2,4]triazolo[4,3-b]pyridazin-6-yl]piperidin-4-ol;
4-[3-(3-fluoro-4-{4-hydroxy-1-[3-(trifluoromethyl)[1,2,4]triazolo[4,3-b]pyridazin-6-yl]piperidin-4-yl}phenoxy)propyl]-1-methylpiperazin-2-one;
4-{4-[2-(4-acetylpiperazin-1-yl)propoxy]phenyl}-1-[3-(trifluoromethyl)[1,2,4]triazolo[4,3-b]pyridazin-6-yl]piperidin-4-ol;
4-{4-[2-(4-acetyl-1,4-diazepan-1-yl)propoxy]phenyl}-1-[3-(trifluoromethyl) [1,2,4]triazolo[4,3-b]pyridazin-6-yl]piperidin-4-ol;
and pharmaceutically acceptable salts thereof.

10. A pharmaceutical composition which comprises a compound according to claim 1 or claim 2, or a pharmaceutically acceptable salt thereof, in association with a pharmaceutically acceptable diluent or carrier.

11. A method of treating prostate cancer in a warm-blooded animal, such as man, in need of such treatment which comprises administering to said animal an effective amount of a compound of the Formula (I), or a pharmaceutically acceptable salt thereof, as defined in claim 1 or claim 2.

12. A process for the preparation of a compound of Formula (I) as defined in claim 1 or 2, or a pharmaceutically acceptable salt thereof, which comprises a process (a), (b), (c), (d), (e), (f) or (g) wherein, unless otherwise defined, the variables are as defined hereinbefore for compounds of Formula (I):
(a) reaction of a compound of Formula (II) with a compound of Formula (III): 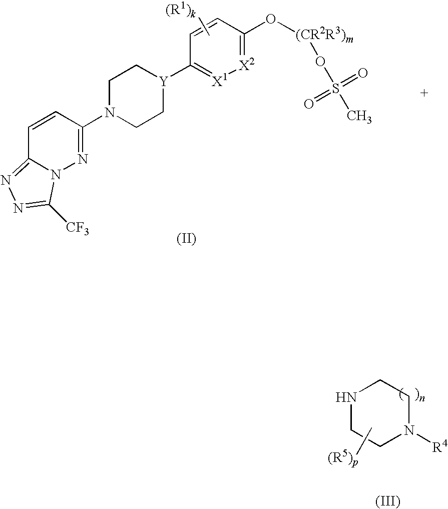
(b) reaction of a compound of Formula (IV) with a compound of Formula (V): 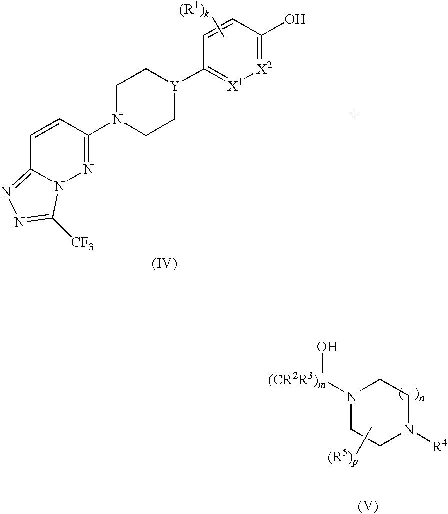
(c) when R⁴ in Formula (I) is C₂₋₆alkanoyl, hydroxyC₂₋₆alkanoyl or C₁₋₆alkoxyC₂₋₆alkanoyl and R⁵ is other than oxo, reaction of a compound of Formula (VI) with the appropriate carboxylic acid, hydroxycarboxylic acid or alkoxycarboxylic acid: 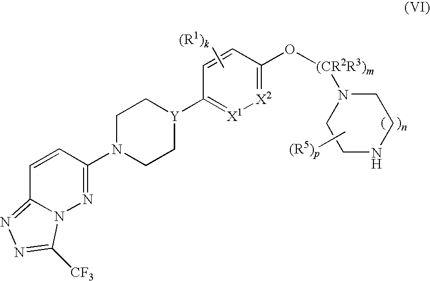
(d) when R⁴ is C₁₋₆alkyl and R⁵ is other than oxo bonded to a ring carbon atom adjacent to the nitrogen atom linked to R⁴, reaction of a compound of Formula (VI) with the appropriate aldehyde, in the presence of a suitable acid and a suitable reducing agent;
(e) when Y is CH, reduction of a compound of Formula (VII): 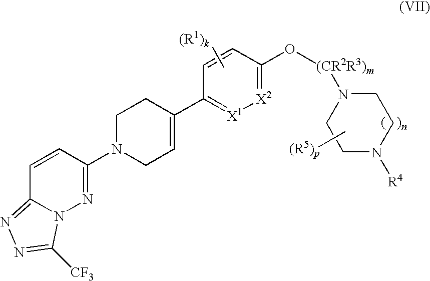
(f) reaction of a compound of Formula (VIII) with a compound of Formula (IX) wherein L represents chloro, bromo or iodo: 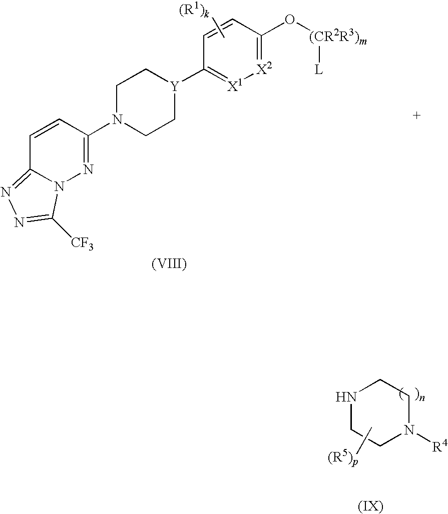
(g) reaction of a compound of Formula (X) with a compound of Formula (XI) wherein L represents chloro, bromo or iodo: 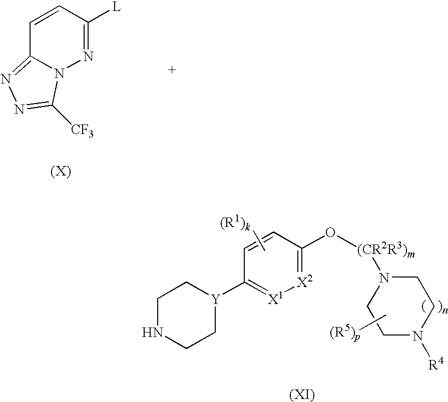
and thereafter, if necessary:
(i) converting a functional group of one compound of the invention into another functional group;
(ii) introducing a new functional group into one compound of the invention;
(iii) removing any protecting groups;
(iv) for compounds of the invention in the form of a single enantiomer separating a racemic compound of the invention into separate enantiomers;
(v) preparing a pharmaceutically acceptable salt thereof and/or
(vi) preparing a crystalline form thereof.
